(19) United States
(12) Patent Application Publication
(10) Pub. No.: US 2024/0041769 A1
(43) Pub. Date: Feb. 8, 2024

(54) COMPOSITIONS AND METHODS FOR DELIVERY OF ANTICANCER AGENTS WITH IMPROVED THERAPEUTIC INDEX

(71) Applicant: NanoTech Pharma Inc., Hillsborough, NJ (US)

(72) Inventors: Fang Liu, Flemington, NJ (US); Xian Xu, Monmouth Junction, NJ (US)

(21) Appl. No.: 18/257,501

(22) PCT Filed: Dec. 14, 2021
(86) PCT No.: PCT/US21/63325

§ 371 Date: Jun. 14, 2023

Related U.S. Application Data
(60) Provisional application No. 63/125,386, filed on Dec. 14, 2020.

(51) Int. Cl. A61K 9/127; A61P 35/00; A61K 31/506

(52) U.S. Cl. CPC A61K 9/1271; A61P 35/00; A61K 31/506

(57) ABSTRACT

Disclosed are liposomal pharmaceutical compositions that can deliver two or more protein kinase inhibitors to function in a synergistic mode for the treatment of cancers. The combination of the protein kinase inhibitors encapsulated in the liposome carriers are useful in achieving desired drug retention, a sustained drug release profile for each therapeutic compound and a synergistic therapeutic effect. Methods for preparing these liposomal pharmaceutical compositions and use of them for treatment of cancers are also disclosed.

### CROSS-REFERENCE TO RELATED APPLICATION

This application claims priority under 35 U.S.C. § 119(e) to U.S. Provisional Patent Application No. 63/125,386, filed on Dec. 14, 2020, the disclosure of which is incorporated herein by reference in its entirety.

### FIELD OF THE DISCLOSURE

The present disclosure relates to pharmaceutical formulations and methods for improved cancer treatment through encapsulation of combined active pharmaceutical ingredients (APIs) in the bilayer and/or aqueous core compartment of liposomes.

### BACKGROUND OF THE DISCLOSURE

Over the past two decades, the protein kinase enzyme family has become one of the most important drug targets for the treatment of various types of human diseases due to their pivotal roles in signal transductions and regulation of a range of cellular activities. More than sixty small molecule protein kinase inhibitors have been approved by the United States Food and Drug Administration (FDA) as therapeutic agents, which target about two dozen different cancer-related protein kinases with advantages in pharmacokinetic properties, costs, patient compliance, and drug storage. Many protein kinase inhibitor-based drug candidates are currently in preclinical or clinical stage of development. However, the rapid development of drug-resistance after a period of clinical use of the protein kinase inhibitors, and their toxicity, severe side effects, and compromised efficacy present critical challenges in both clinical and experimental oncology and remain major problems. To overcome these challenges in the clinical assessments, a combinational therapy that combines two or more protein kinase inhibitors is a cornerstone of cancer treatment, particularly in hope of evading the treatment-related drug resistances and improving efficacy compared to the mono-therapy approach. Drug resistance to protein kinase inhibitors is a common problem especially in the metastatic stage after patients typically have had exposure to multiple lines of prior therapies. Due to the development of drug resistance, patients can experience a rapid disease progression during or shortly after the completion of treatment. This resistance results in a limited number of treatment choices for patients. Therefore, to minimize the impact of drug resistance, concurrent combined use of two or more anticancer drugs with unrelated mechanisms of action and differing modes of drug resistance has been attempted.

Targeted therapies have the advantages to a chemotherapy in their ability to actively target specific cellular receptors. The conventional chemotherapy does not discriminate effectively between tumor cells bur rapidly divides normal cells, thus leading to nonspecific adverse effects. In contrast, target-specific anticancer therapies interfere with molecular targets that have an important role in tumor growth or progression distinct from normal cells. In addition, some of those agents act as inhibitors to multiple drug resistance (MDR) related proteins, thereby increasing the response rate. Overall targeted therapies provide a broader therapeutic window with less toxicity and higher response rate compared to the conventional chemotherapy.

During the recent years, combination of the protein kinase inhibitors has been widely exploited for enhanced cancer treatment in clinic. However, the traditional cocktail administration of combination regimens often suffers from varying pharmacokinetics among different drugs. In the common preclinical and clinical practices, the combined protein kinase inhibitor drug therapy was administrated singularly with various amounts and/or at different dosing schedules without pharmaceutical preparations designed to control delivery or half-lives of the drugs. These administration methods have various drawbacks that limit the therapeutic use of the combined drug treatment. Typically, the components of such regimens are first developed individually, without consideration of the many issues that may arise when they are used in combination, such as on-target antagonism and potentiation of adverse events.

Although beneficial therapeutic effectiveness from combination treatment is promising when considering the theoretically nonoverlapping mechanisms of action of each anticancer drug, the above common combination in clinical cancer treatment is far from perfect, typically with moderate enhanced efficacy and additive toxicity. Therefore, various approaches have been investigated by incorporating nanotechnology with combination anticancer treatment, based on the hypothesis that by delivering two or more drugs simultaneously using a carrier-mediated drug delivery system, the combination of the drug delivery system could generate synergistic anticancer effects, reduce individual drug related toxicity, control drug release, and/or unify the pharmacokinetics of each drug. Liposomes, dendrimers, polymeric nanoparticles, and water-soluble polymer-drug conjugates have been reported as carriers for the delivery of multiple drug cocktails in recent years. (Markman, J. L., et al., Adv. Drug Delivery Rev. 2013, 65, 1866-1879).

Among the nanocarriers, the lipid-based nanoparticles have demonstrated excellent outcomes by overcoming P-gp mediated efflux, sequestering drugs at tumor sites via enhanced permeability and retention (EPR) effect, and escaping endosomal clearance once internalized. For example, CPX-351 (Vyxeos©) is a dual-drug liposomal formulation based on the encapsulation of cytarabine and daunorubicin that was rationally designed to improve efficacy over the traditional 7+3 cytarabine/daunorubicin chemotherapy regimen for patients with acute myeloid leukemia (AML) (Lawrence D Mayer, et al., International Journal of Nanomedicine 2019:14, 3819-3830). To fulfill the effective delivery of multiple protein kinase inhibitors for cancer treatment, some studies have been attempted to encapsulate such drugs into a liposome-based delivery vehicle, which is designed to shield the drug from mechanisms that would otherwise result in their fast clearance from the bloodstream. However, innovative liposome-based delivery systems for combined protein kinase inhibitor delivery are still much needed to improve drug delivery specificity, achieve synergistic therapeutic effects, reduce drug resistance and drug-related adverse effects, and overall enhance the drug therapeutic index.

### SUMMARY OF THE DISCLOSURE

This disclosure provides pharmaceutical compositions as a liposome-based drug delivery system and methods for administering an effective amount of one, two, or more protein kinase inhibitors (e.g., afatinib, nintedanib, abemaciclib, sunitinib, crizotinib, dasatinib, ceritinib, osimertinib, ponatinib, ruxolitinib, or others) to patients using the liposome-based drug delivery system. Two or more protein kinase inhibitors can be encapsulated either into the aqueous core compartment of the liposome (e.g., for hydrophilic, water-soluble inhibitors) or into the lipid bilayer of the liposome (e.g., for lipophilic, poorly water-soluble inhibitors).

In some embodiments, one single liposome vehicle can carry both lipophilic and hydrophilic protein kinase inhibitors, where the hydrophilic inhibitors are in the aqueous core and the lipophilic inhibitors are in the lipid bilayer. These compositions allow two or more protein kinase inhibitors to be delivered to the disease site in a coordinated fashion, thereby ensuring that the protein kinase inhibitors can be presented at the disease site with a desired amount or ratio. Combined delivery of two or more protein kinase inhibitors can be achieved either by co-encapsulating them within one lipid-based delivery vehicle as mentioned above, or by encapsulating each inhibitor into a separate lipid-based delivery vehicle. In the latter case, the pharmacokinetics (PK) of the composition is controlled by the lipid-based delivery vesicles themselves such that a coordinated delivery is achieved (provided that the PK of the delivery systems are comparable).

In one aspect, the present disclosure provides a pharmaceutical composition comprising liposomes suspended in a liquid medium that contains water and a buffering agent to maintain pH. The liposome comprises an interior aqueous compartment surrounded by an outer lipid bilayer membrane. The lipid bilayer membrane contains a hydrophilic inner surface forming the interior compartment, a lipophilic bilayer, and a hydrophilic outer surface in contact with the liquid medium of the composition. Three main scenarios regarding the locations of the protein kinase inhibitor(s) within the liposome formulation are notable. Scenario I: The interior aqueous compartment contains one, two, or more hydrophilic protein kinase inhibitors. Scenario II: The lipophilic bilayer contains one, two, or more lipophilic protein kinase inhibitors. Scenario III: The interior aqueous compartment contains one or more hydrophilic protein kinase inhibitors and in the same liposome, the lipophilic bilayer contains one or more lipophilic protein kinase inhibitors. For all the three scenarios above, the co-encapsulated protein kinase inhibitors can be released from the liposome and induce synergistic therapeutic effect.

In another aspect, the present disclosure provides a liposome composition for parenteral administration comprising one, two, or more protein kinase inhibitors encapsulated inside the liposomes at therapeutically effective ratios, especially those that are non-antagonistic.

In another aspect, the present disclosure provides a pharmaceutical composition according to any embodiments disclosed herein for use in the treatment of a cancer, or drug resistance and side effects of a cancer drug in a subject in need of treatment, wherein the cancer is selected from breast cancer, melanoma, gastrointestinal cancer, lung cancer, colorectal cancer, Ewing sarcoma, pancreatic cancer, prostate cancer, bladder cancer, kidney cancer, thyroid cancer, uterine cancer, and gastrointestinal stromal tumors, or the like, wherein the protein kinase inhibitors are delivered by the liposome with a synergistic cytotoxic or cytostatic effect on cancer cells.

In another aspect, the present disclosure provides a method of treating a cancer or drug resistance of a cancer and reducing toxicity and side effects of cancer drug. The disclosure includes the administering to a subject in need of treatment by a therapeutically effective amount of a pharmaceutical composition according to any embodiments disclosed herein.

In another aspect, the present disclosure provides a method of preparing a liposomal pharmaceutical composition, wherein the liposome is made by a process comprising active drug loading, or passive drug loading, or the sequential drug loading by coupling passive drug loading and then active drug loading together.

In another aspect, the present disclosure provides a treatment kit comprising a container and a plurality of the drug-loaded liposomes according to any embodiments disclosed herein in the container, wherein the drug-loaded liposomes can be suspended in a sterile diluent solution ready for administration to a subject in need of treatment.

In another embodiment, a liposomal composition comprises two or more protein kinase inhibitors in a molar ratio between the combined protein kinase inhibitor agents which exhibits a desired biologic effect to relevant cells in culture and tumor homogenates. Preferably, the molar ratio is that at which the agents are non-antagonistic.

In another embodiment, this disclosure provides a method to deliver a therapeutically effective amount of the combined protein kinase inhibitors (e.g., afatinib/nintedanib, afatinib/dasatinib, afatinib/ceritinib, abemaciclib/sunitinib, osimertinib/afatinib, osimertinib/crizotinib, ceritinib/dasatinib, or others) to a desired target (e.g., tumor site) by administering the compositions of the invention.

In another embodiment, this disclosure provides a method to deliver a therapeutically effective amount of the combination of protein kinase inhibitors by administering a protein kinase inhibitor stably associated with a first delivery vehicle and another kinase inhibitor stably associated with a second delivery vehicle. The first and second delivery vehicles may be contained in separate vials, the contents of the vials being administered to a patient simultaneously or sequentially. In one embodiment, the molar ratio of the combined protein kinase inhibitors is non-antagonistic.

In another embodiment, this disclosure provides a method to prepare a therapeutic composition comprising liposomes containing a ratio of two or more protein kinase inhibitors to achieve a desired therapeutic effect. The method comprises: (a) providing a panel of two or more protein kinase inhibitors, wherein the panel comprises at least one, but preferably a multiplicity of ratios of the drugs; (b) testing the ability of the members of the panel to exert a biological effect on a relevant cell culture or tumor homogenate over a range of concentrations; (c) selecting a member of the panel where in the ratio provides a desired therapeutic effect on the cell culture and tumor homogenate over a suitable range of concentrations; and (d) stably associating the ratio of drugs represented by the successful member of the panel into lipid-based drug delivery vehicles. In some embodiments, sometimes preferably, the abovementioned desired therapeutic effect is non-antagonistic.

As further described below, in designing an appropriate combination in accordance with the method described above, sometimes preferably, the non-antagonistic ratios are selected as those that have a combination index CI≤1.1 (equal to or smaller than 1.1). Specifically, a CI<0.9 indicates a synergistic effect of the drug combinations, where a range of 0.9≤CI≤1.1 is considered to be an additive effect, and CI>1.1 is considered to be antagonism of the drug combinations.

In further embodiments, suitable liposomal formulations are designed such that they stably incorporate an effective amount of a combination of two or more protein kinase inhibitors and allow for the sustained release of the combined drugs in-vivo. Sometimes preferably, the formulations contain pegylated mPEG-DSPE or at least one negatively charged lipid, such as phosphatidylglycerol (DSPG).

In further embodiments, liposomes can be prepared with active drug loading, passive drug loading, or sequential combination of passive and then active drug loading process from natural phospholipids and synthetic analogues such as the electrical charge zwitterionic phosphatidylcholines, or the like. Minor proportions of anionic phospholipids, such as phosphatidylglycerols, can be added to generate a net negative surface charge for colloid stabilization. For the active drug loading method, various trapping agents are used based on the physical properties of the co-loaded drugs (e.g., ammonium sulfate, transition metal ions, and ammonium or substituted ammonium salts of the following: polyanionized sulfated cyclodextrin, sulfobutyl ether cyclodextrin, polyanionized sulfated sugar, polyphosphate, and the like).

The encapsulation of two or more kinase inhibitor-based anticancer drugs in liposomes is a novel approach to stimulate the cross-talking among multiple signaling pathways that contribute to the growth and development of tumors. The combination of the kinase inhibitors is a useful therapeutic method for the treatment of various types of human disease due to its pivotal roles in signal transductions and regulation of a range of cellular activities.

In another embodiment, the present disclosure provides liposomes as disclosed in any embodiment examples herein and/or prepared by a method according to any embodiments or examples disclosed herein.

Other aspects or advantages of the disclosure will be better appreciated in view of the following detailed description, examples, and claims.

### BRIEF DESCRIPTION OF THE DRAWINGS

FIG. 1 illustrates chemical structures of some example protein kinase inhibitors. A: Afatinib dimaleate; B: Nintedanib esylate; C: Abemaciclib mesylate; D: Sunitinib malate; E: Crizotinib; F: Osimertinib mesylate; G: Dasatinib monohydrate; H: Ceritinib.

FIG. 2 illustrates the effect of drug to lipid ratio (w/w, total lipid concentration is fixed at 8 mg/mL) on encapsulation efficiency (EE %) of PEGylated AFA-L with ammonium sulfate (AS), TEA-SBE-β-CD, and TEA-SOS used as the trapping agent.

FIG. 3 illustrates the effect of drug to lipid ratio (w/w, total lipid concentration is fixed at 8 mg/mL) on nintedanib encapsulation efficiency (EE %) of PEGylated NIN-L with ammonium sulfate (AS), TEA-SBE-β-CD, and TEA-SOS used as the trapping agents.

FIG. 4 illustrates the structures of trapping agents TEA-SBE-β-CD and TEA-SOS.

FIG. 5 illustrates an example of combined anti-cancer protein kinase inhibitors co-loaded within PEGylated liposomes. A total of three scenarios on the location of the anti-cancer inhibitors within the liposome are illustrated: Scenario I: both of the hydrophilic (water-soluble) anti-cancer inhibitors are loaded within the aqueous core of the liposome; Scenario II: one hydrophilic (water-soluble) anti-cancer inhibitor is loaded in the aqueous core and another lipophilic (poorly water-soluble) anti-cancer inhibitor is encapsulated in the lipid bilayer; Scenario III: both of the lipophilic (poorly water-soluble) anti-cancer inhibitors are loaded within the lipid bilayer of the liposome.

FIG. 6 illustrates the particle size distribution (Intensity %) of PEGylated AFA-L, NIN-L and AFA/NIN-L at 1:10, 1:5, 1:1 AFA to NIN molar ratios. (NH₄)₂SO₄ was used as the trapping agent for all liposome drug product above.

FIG. 7 illustrates the particle size distribution (Intensity %) of PEGylated AFA-L, NIN-L and AFA/NIN-L at 1:10, 1:5, 1:1 AFA to NIN molar ratios. TEA-SBE-β-CD was used as the trapping agent for all liposome drug product above.

FIG. 8 illustrates Cryo-Transmission Electron Microscopy (Cryo-TEM) images of A: AFA/NIN-L; B: OSI/AFA-L, and C: DAS/CER-L. Both of AFA/NIN-L and OSI/AFA-L are PEGylated liposomes with TEA-SOS used as the trapping agent. DAS/CER-L is based on the DSPG liposome. The molar ratio of AFA/NIN, OSI/AFA and DAS/CER used in the liposome are 1 to 5, 1 to 1, and 1.8 to 1, respectively.

FIG. 9A, FIG. 9B, and FIG. 9C (collectively FIG. 9) illustrates in-vitro dissolution profiles of protein kinase inhibitor loaded liposomes. The dissolution study was performed at 45° C. accelerated conditions. A: Dissolution profile of AFA and/or NIN loaded PEGylated liposomes. The following liposome drug product were studied: (1) AFA only loaded liposome with ammonium sulfate (AS) used as the trapping agent (AFA-L-AS, solid square); (2) NIN only loaded liposome with AS used as the trapping agent (NIN-L-AS, blank triangle); (3) AFA and NIN co-loaded liposome with AS used as the trapping agent (AFA/NIN-L-AS, solid triangle for AFA, blank diamond for NIN) and (4) AFA and NIN co-loaded liposome with TEA-SBE-β-CD used as the trapping agent (AFA/NIN-L-CD, solid circle for AFA and blank square for NIN). For the co-loaded liposomes, the molar ratio of AFA to NIN was prepared as 1:5. B: Dissolution profile of ABE and SUN (molar ratio of 1:5) co-loaded PEGylated liposomes. The following drug products were studied: (1) co-loaded ABE/SUN using TEA-SBE-β-CD as the trapping agent (solid circle for SUN and solid square for ABE) (2) co-loaded ABE/SUN using Tris-SBE-β-CD as the trapping agent (solid triangle for SUN and solid diamond for ABE). C: Dissolution profile of AFA and CRI (molar ratio of 1:1) from co-loaded PEGylated liposomes using TEA-SBE-β-CD as the trapping agent. The following two liposome drug products were studied: (1) co-loaded AFA/CRI liposome with a lipid composition that contains 74 wt % of DSPC (solid square for AFA and solid triangle for CRI). (2) co-loaded AFA/CRI liposome with a lipid composition that contains 68 wt % of DSPC (blank square for AFA and blank triangle for CRI).

FIG. 10 illustrates the physical stability of AFA/DAS co-loaded liposome analyzed by dynamic light scattering. Samples were stored at 2-8° C. and mean particle size (nm) and polydispersity (PDI) were measured at pre-determined time points. TEA-SBE-β-CD was used as the trapping agent for active loading of AFA. The molar ratio of AFA to DAS in the liposome is 3 to 1.

FIG. 11 illustrates the physical stability of CER/DAS co-loaded liposomes analyzed by dynamic light scattering. Samples were stored under 2-8° C., and the mean particle size and polydispersity (PDI) were measured at pre-determined time points.

FIG. 12A and FIG. 12B (collectively FIG. 12) illustrate in-vitro evaluation of afatinib (AFA) and nintedanib (NIN) for synergistic mode in HT-29 colorectal cells. A: In-vitro evaluation of afatinib and nintedanib for synergistic mode in HT-29 colorectal cells as a function of afatinib/nintedanib ratio and drug concentration. Concentrations of the fixed afatinib/nintedanib molar ratios were designed to provide a broad range of cell growth inhibition (indicated by Fa). Representative plot of combination index (CI) value as a function of cell growth inhibition at fixed drug ratios is shown, where CI values of <1, ˜1, and >1 indicate synergy, additivity, and antagonism, respectively. B: In-vitro evaluation of CI from HT-29 colorectal cells plotted at ED₇₅ (black column, Fa=0.75) and ED₉₀ (gray columns, Fa=0.90) as a function of afatinib/nintedanib molar ratio (n=3 independent repeats).

FIG. 13A and FIG. 13B (collectively FIG. 13) illustrate in-vitro evaluation of afatinib (AFA) and nintedanib (NIN) for synergistic mode in H1975 non-small cell lung cancer (NSCLC) cells. A: Representative plot of combination index (CI) value as a function of cell growth inhibition (indicated by Fa) at different AFA to NIN molar ratios, where CI values of <1, ˜1, and >1 indicate synergy, additivity, and antagonism, respectively. B: In-vitro evaluation of CI based on H1975 NSCLC plotted at ED₇₅ (black column, Fa=0.75) and ED₉₀ (white columns, Fa=0.90) as a function of different AFA/NIN molar ratios (n=3 independent repeats).

FIG. 14A and FIG. 14B (collectively FIG. 14) illustrate in-vitro evaluation of abemaciclib (ABE) and sunitinib (SUN) for synergistic mode in 786-O renal cancer cells. A: Representative plot of combination index (CI) value as a function of cell growth inhibition (indicated by Fa) at different ABE to SUN molar ratios, where CI values of <1, ˜1, and >1 indicate synergy, additivity, and antagonism, respectively. B: In-vitro evaluation of CI based on 786-O renal cancer cells plotted at ED₇₅ (black column, Fa=0.75) and ED₉₀ (white columns, Fa=0.90) as a function of different ABE/SUN molar ratios (n=3 independent repeats).

FIG. 15A and FIG. 15B (collectively FIG. 15) illustrate in-vitro evaluation of abemaciclib (ABE) and sunitinib (SUN) for synergistic mode in Caki-1 renal cancer cells. A: Representative plot of combination index (CI) value as a function of cell growth inhibition (indicated by Fa) at different ABE to SUN molar ratios, where CI values of <1, ˜1, and >1 indicate synergy, additivity, and antagonism, respectively. B: In-vitro evaluation of CI based on Caki-1 renal cancer cells plotted at ED₇₅ (black column, Fa=0.75) and ED₉₀ (white columns, Fa=0.90) as a function of different ABE/SUN molar ratios (n=3 independent repeats).

FIG. 16A and FIG. 16B (collectively FIG. 16) illustrate in-vitro evaluation of afatinib (AFA) and crizotinib (CRI) for synergistic mode in MSTO-211H mesothelioma cells. A: Representative plot of combination index (CI) value as a function of cell growth inhibition (indicated by Fa) at different AFA to CRI molar ratios, where CI values of <1, ˜1, and >1 indicate synergy, additivity, and antagonism, respectively. B: In-vitro evaluation of CI based on MSTO-211H cells plotted at ED₇₅ (black column, Fa=0.75) and ED₉₀ (white columns, Fa=0.90) as a function of different AFA/CRI molar ratios (n=3 independent repeats).

FIG. 17A and FIG. 17B (collectively FIG. 17) illustrate in-vitro evaluation of afatinib (AFA) and crizotinib (CRI) for synergistic mode in H1975 NSCLC cells. A: Representative plot of combination index (CI) value as a function of cell growth inhibition (indicated by Fa) at different AFA to CRI molar ratios, where CI values of <1, ˜1, and >1 indicate synergy, additivity, and antagonism, respectively. B: In-vitro evaluation of CI based on H1975 NSCLC cells plotted at ED₇₅ (black column, Fa=0.75) and ED₉₀ (white columns, Fa=0.90) as a function of different AFA/CRI molar ratios (n=3 independent repeats).

FIG. 18A and FIG. 18B (collectively FIG. 18) illustrate in-vitro evaluation of osimertinib (OSI) and afatinib (AFA) for synergistic mode in H1975 NSCLC cells. A: Representative plot of combination index (CI) value as a function of cell growth inhibition (indicated by Fa) at different OSI to AFA molar ratios, where CI values of <1, ˜1, and >1 indicate synergy, additivity, and antagonism, respectively. B: In-vitro evaluation of CI based on H1975 NSCLC cells plotted at ED₇₅ (black column, Fa=0.75) and ED₉₀ (white columns, Fa=0.90) as a function of different OSI/AFA molar ratios (n=3 independent repeats).

FIG. 19A and FIG. 19B (collectively FIG. 19) illustrate in-vitro evaluation of osimertinib (OSI) and afatinib (AFA) for synergistic mode in HCC827 NSCLC cells. A: Representative plot of combination index (CI) value as a function of cell growth inhibition (indicated by Fa) at different OSI to AFA molar ratios, where CI values of <1, ˜1, and >1 indicate synergy, additivity, and antagonism, respectively. B: In-vitro evaluation of CI based on HCC827 NSCLC cells plotted at ED₇₅ (black column, Fa=0.75) and ED₉₀ (white columns, Fa=0.90) as a function of different OSI/AFA molar ratios (n=3 independent repeats).

FIG. 20A and FIG. 20B (collectively FIG. 20) illustrate In-vitro evaluation of crizotinib (CRI) and osimertinib (OSI) for synergistic mode in H1975 NSCLC cells. A: Representative plot of combination index (CI) value as a function of cell growth inhibition (indicated by Fa) at different CRI to OSI molar ratios, where CI values of <1, ˜1, and >1 indicate synergy, additivity, and antagonism, respectively. B: In-vitro evaluation of CI based on H1975 NSCLC cells plotted at ED₇₅ (black column, Fa=0.75) and ED₉₀ (white columns, Fa=0.90) as a function of different CRI/OSI molar ratios (n=3 independent repeats).

FIG. 21A and FIG. 21B (collectively FIG. 21) illustrate in-vitro evaluation of crizotinib (CRI) and osimertinib (OSI) for synergistic mode in HCC827 NSCLC cells. A: Representative plot of combination index (CI) value as a function of cell growth inhibition (indicated by Fa) at different CRI to OSI molar ratios, where CI values of <1, ˜1, and >1 indicate synergy, additivity, and antagonism, respectively. B: In-vitro evaluation of CI based on HCC827 NSCLC cells plotted at ED₇₅ (black column, Fa=0.75) and ED₉₀ (white columns, Fa=0.90) as a function of different CRI/OSI molar ratios (n=3 independent repeats).

FIG. 22A and FIG. 22B (collectively FIG. 22) illustrate in-vitro evaluation of afatinib (AFA) and dasatinib (DAS) for synergistic mode in H1975 NSCLC cells. A: Representative plot of combination index (CI) value as a function of cell growth inhibition (indicated by Fa) at different AFA to DAS molar ratios, where CI values of <1, ˜1, and >1 indicate synergy, additivity, and antagonism, respectively. B: In-vitro evaluation of CI based on H1975 NSCLC cells plotted at ED₇₅ (black column, Fa=0.75) and ED₉₀ (white columns, Fa=0.90) as a function of different AFA/DAS molar ratios (n=3 independent repeats).

FIG. 23A and FIG. 23B (collectively FIG. 23) illustrate in-vitro evaluation of afatinib (AFA) and dasatinib (DAS) for synergistic mode in HCC827 NSCLC cells. A: Representative plot of combination index (CI) value as a function of cell growth inhibition (indicated by Fa) at different AFA to DAS molar ratios, where CI values of <1, ˜1, and >1 indicate synergy, additivity, and antagonism, respectively. B: In-vitro evaluation of CI based on HCC827 NSCLC cells plotted at ED₇₅ (black column, Fa=0.75) and ED₉₀ (white columns, Fa=0.90) as a function of different AFA/DAS molar ratios (n=3 independent repeats).

FIG. 24A and FIG. 24B (collectively FIG. 24) illustrate in-vitro evaluation of dasatinib (DAS) and ceritinib (CER) for synergistic mode in H1975 NSCLC cells. A: Representative plot of combination index (CI) value as a function of cell growth inhibition (indicated by Fa) at different DAS to CER molar ratios, where CI values of <1, ˜1, and >1 indicate synergy, additivity, and antagonism, respectively. B: In-vitro evaluation of CI based on H1975 NSCLC cells plotted at ED₇₅ (black column, Fa=0.75) and ED₉₀ (white columns, Fa=0.90) as a function of different DAS/CER molar ratios (n=3 independent repeats).

FIG. 25A and FIG. 25B (collectively FIG. 25) illustrate in-vitro evaluation of dasatinib (DAS) and ceritinib (CER) for synergistic mode in HCC827 NSCLC cells. A: Representative plot of combination index (CI) value as a function of cell growth inhibition (indicated by Fa) at different DAS to CER molar ratios, where CI values of <1, ˜1, and >1 indicate synergy, additivity, and antagonism, respectively. B: In-vitro evaluation of CI based on HCC827 NSCLC cells plotted at ED₇₅ (black column, Fa=0.75) and ED₉₀ (white columns, Fa=0.90) as a function of different DAS/CER molar ratios (n=3 independent repeats).

FIG. 26A and FIG. 26B (collectively FIG. 26) illustrate pharmacokinetic studies on liposomes co-loaded with AFA/NIN (AFA/NIN molar ratio of 1:5). AFA/NIN-L was administrated at 7.0 (AFA free base)/38.9 (NIN free base) mg/kg to BALB/c nude female mice (n=3 per time point) upon intravenous injection. The drug product contains TEA-SOS as the trapping agent. Circulating plasma AFA to NIN molar ratios at each time point were calculated from absolute plasma concentrations. A: Plasma drug concentration over time. B: AFA to NIN molar drug ratio in plasma over time.

FIGS. 27A, 27B and 27C (collectively FIG. 27) illustrate in-vivo efficacy of afatinib (AFA) and nintedanib (NIN) co-delivered by liposomes against cell line-derived xenograft model based on H1975 NSCLC cell. A: Tumor volume change overtime. Inset: Tumor growth over time for mice treated by Free AFA/NIN cocktail, NIN-L and AFA/NIN-L. B: body weight change overtime. C: Photo of representative tumor xenograft from each group which was dissected at the completion of the study. Tumor bearing female BALB/c nude mice (n=6 per group) were treated intravenously every two days for a total of 20 days. Information on each treatment group is described as follows: Saline (blank triangle); Free AFA solution (7.0 mg/kg, blank diamond); Free combo of AFA and NIN (7.0 mg/kg AFA and 38.9 mg/kg NIN, blank square); Liposome AFA-L (7.0 mg/kg, solid diamond); liposome NIN-L (38.9 mg/kg, solid circle) and liposome combo of AFA/NIN-L (7.0 mg/kg AFA and 38.9 mg/kg NIN, solid square). For all liposomal drug products, TEA-SOS was used as the intraliposomal trapping agent. For AFA/NIN-L, the AFA to NIN molar drug ratio is 1 to 5.

FIGS. 28A, 28B and 28C (collectively FIG. 28) illustrate in-vivo efficacy of afatinib (AFA) and nintedanib (NIN) co-delivered by liposomes against cell line-derived xenograft model based on HT-29 colorectal cancer cell. A: tumor volume change overtime. Inset: Tumor growth over time for mice treated by Free AFA/NIN cocktail, NIN-L and AFA/NIN-L. B: body weight change overtime. C: Photo of representative tumor xenograft from each group which was dissected at the completion of the study. Tumor bearing female BALB/c nude mice (six per group) were treated intravenously every two days for a total of 19 days. Information on each treatment group is described as follows: Saline (blank triangle); Free AFA solution (7.0 mg/kg, blank diamond); Free combo of AFA and NIN (7.0 mg/kg AFA and 38.9 mg/kg NIN, blank square); Liposome AFA-L (7.0 mg/kg, solid diamond); liposome NIN-L (38.9 mg/kg, solid circle) and liposome combo of AFA/NIN-L (7.0 mg/kg AFA and 38.9 mg/kg NIN, solid square). For all liposomal drug products, TEA-SOS was used as the intraliposomal trapping agent. For free AFA/NIN cocktail solution and AFA/NIN-L, the AFA to NIN molar drug ratio is 1 to 5.

### DETAILED DESCRIPTION OF THE DISCLOSURE

The rationale for employing combination drug therapy is synergistic drug interaction. First, when multiple drugs with different molecular targets are applied, the cancer adaptation process such as cancer cell mutations can be delayed. Second, when multiple drugs target different cellular pathways, they could function synergistically for higher therapeutic efficacy and higher target selectivity. Currently available combination regimens for multiple cancers in clinical studies are very much limited to administrating a physical mixture of two or more anticancer agents. The common clinically used combination regimens in clinical studies can be generally classified based on their mechanisms of action, including: (1) combination of nonspecific small molecule chemotherapeutic agents, (2) combination of specific cellular receptor targeted agents and chemotherapeutic agents, and (3) combination of specific cellular receptor targeted agents (e.g., small molecule protein kinase inhibitor, macromolecule antibodies, nucleic acids, etc.).

In the present disclosure, we have identified the drug delivery formulations required to accommodate one, two or more protein kinase inhibitors for cancer treatment and cancer drug resistance prevention (e.g., afatinib, nintedanib, abemaciclib, sunitinib, crizotinib, dasatinib, osimertinib, ceritinib, ruxolitinib, or the like). Such formulations containing drug combinations result in improved therapeutic index, reduced drug resistance and side effects, superior drug retention in the carrier, which results in prolonged blood circulation time and sustained release of each agent. We further demonstrated that synergistic ratios of these drugs, when encapsulated in liposomes, can be successfully maintained in the blood compartment overtime, which results in enhanced efficacy as compared to the combination of drugs in their conventional dosage forms (e.g., tablet or simple injectable).

The disclosure provides compositions comprising liposomes encapsulating one, two or more protein kinase inhibitors, wherein the combination of the selected protein kinase inhibitors is present at molar ratios, that exhibit a desired cytotoxic, cytostatic or biologic effect to relevant cells or tumor homogenates. Sometimes preferably, liposomal compositions provided herein include liposomes loaded with two kinase inhibitors at a molar ratio which exhibits a non-antagonistic effect to relevant cells or tumor homogenates.

In one aspect, the present disclosure provides a pharmaceutical composition comprising liposomes suspended in a liquid medium, wherein the liquid medium contains water and a pH buffering agent; wherein the liposome contains an interior compartment surrounded by an outer lipid bilayer membrane, wherein the interior aqueous compartment contains the hydrophilic protein kinase inhibitors in an aqueous medium; wherein the lipid bilayer membrane contains a hydrophilic inner surface forming the interior compartment, a lipophilic bilayer, and a hydrophilic outer surface in contact with the liquid medium of the composition; and wherein the lipid bilayer membrane contains hydrophobic protein kinase inhibitors, and the encapsulated multiple protein kinase inhibitors inside liposome can be released in a synergistic or additive mode.

In one embodiment, in the pharmaceutical composition, the aqueous interior compartment of the liposomes further comprises a trapping agent. The trapping agent is selected from ammonium sulfate, ammonium or substituted ammonium salts of polyanionized sulfobutyl ether cyclodextrin (e.g., TEA-SBE-α-cyclodextrin, TEA-SBE-β-cyclodextrin, TEA-SBE-γ-cyclodextrin, Tris-SBE-α-cyclodextrin, Tris-SBE-β-cyclodextrin and Tris-SBE-γ-cyclodextrin); ammonium or substituted ammonium salts of polyanionized sulfated carbohydrates (e.g., TEA-SOS and Tris-SOS); ammonium or substituted ammonium salts of polyphosphate (e.g., triethylammonium inositol hexaphosphate and tris-hydroxymethyl aminomethane inositol hexaphosphate); transition metal salts (e.g., salts of copper, zinc, manganese, nickel, cobalt, or the like, with halide, sulfate or gluconate counterions); quaternary ammonium compounds (e.g., benzalkonium chloride, benzethonium chloride, cetrimonium bromide, stearyl dimethylbenzyl ammonium chloride), polyoxyethylene (i.e., polyethylene glycols), and coconut amine.

In another embodiment, in the pharmaceutical composition, the two kinase inhibitors are encapsulated at a molar ratio in the range of about 60:1 to about 1:60 mole/mole, sometimes preferably about 30:1 to about 1:30, and sometimes more preferably about 10:1 to about 1:10, about 5:1 to about 1:5, about 3:1 to about 1:3, about 2:1 to about 1:2, or about 1:1.

In another embodiment, in the pharmaceutical composition, (a) exterior bilayer membrane comprises one or more phospholipids; and (b) the outer surface of the bilayer membrane is modified with a surface-modifying agent selected from polyethylene glycols, charged lipids, and combinations thereof.

In another embodiment, in the pharmaceutical composition, the pH of the liposome suspension is in the range of about 5-8. The aqueous medium further comprises one or more of the following: water, buffering agent, a dispersion medium, and optionally comprises isotonic agents, for example, sucrose, mannitol, sodium chloride, or the like.

In another embodiment, in the pharmaceutical composition, the liposome comprises a lipid selected from phospholipids (e.g., HSPC, DSPC, DDPC, DEPC, DLPC, DMPC, DPPC, PSPC, SMPC, SOPC, SPPC, phosphatidylglycerol, phosphatidylinositol, glyceroglycolipids, sphingoglycolipids), sterols, and derivatives thereof.

In another embodiment, in the pharmaceutical composition, the sterol comprises about 0-60% mole of total lipids.

In another embodiment, in the pharmaceutical composition, the liposomes have a mean particle size (diameter) between 4.5 nm to 450 nm, sometimes preferably between 25 nm and 300 nm, and sometimes more preferably between 50 nm to 200 nm.

In another embodiment, in the pharmaceutical composition, the lipid bilayer membrane of the liposome comprises (a) at least 10 mol % of total lipids of a phospholipid selected from phosphatidylcholine (0-80% mole of total lipid, e.g., HSPC, DSPC, DPPC, DMPC), phosphatidylglycerol (0-70% mole of total lipid, e.g., DSPG), phosphatidylinositol, glyceroglycolipids, sphingoglycolipids (e.g., sphingomyelin), and combinations thereof; (b) 0-60 mol % of total lipids of sterol, prefer cholesterol or a derivative thereof; and (c) optionally a charged phospholipid derivatized with polyethylene glycol 0-10% mole of total lipid (e.g., mPEG-2000-DSPE).

In another embodiment, in the pharmaceutical composition, the outer surface of the lipid bilayer membrane of the liposomes comprises a surface negative charged lipid (e.g. DSPG) or a surface-modifying agent containing polyethylene glycol (e.g., mPEG-2000-DSPE), wherein the molar ratio of the total lipid to the total encapsulated kinase inhibitors, is at least equivalent (1:1).

In another embodiment, in the pharmaceutical composition, the protein kinase inhibitor is selected from acalabrutinib, abemaciclib, afatinib, aflibercept, alectinib, avapritinib, axitinib, baricitinib, brigatinib, binimetinib, bosutinib, cabozantinib, capmatinib, ceritinib, cobimetinib, crizotinib, dabrafenib, dacomitinib, dasatinib, encorafenib, entrectinib, erdafitinib, erlotinib, everolimus, fedratinib, fostamatinib, gefitinib, gilteritinib, ibrutinib, icotinib, imatinib, lapatinib, larotrectinib, lenvatinib, lorlatinib, midostaurin, neratinib, nilotinib, nintedanib, netarsudil, osimertinib, pacritinib, pazopanib, pexidartinib, pemigatinib, palbociclib, ponatinib, pexidartinib, ponatinib, pralsetinib, quizartinib, regorafenib, ribociclib, ripretinib, ruxolitinib, selpercatinib, selumetinib, sorafenib, sunitinib, temsirolimus, tofacitinib, trametinib, tucatinib, upadacitinib, vandetanib, vemurafenib, zanubrutinib, and ziv-aflibercept, and more, or combinations thereof.

In another embodiment, in the pharmaceutical composition, the liposome comprises the example protein kinase inhibitors selected from the following:
(a) afatinib or nintedanib encapsulated alone;
(b) afatinib and nintedanib co-encapsulated;
(c) abemaciclib or sunitinib encapsulated alone;
(d) abemaciclib and sunitinib co-encapsulated;
(e) dasatinib or afatinib encapsulated alone;
(f) dasatinib and afatinib co-encapsulated;
(g) ceritinib or afatinib encapsulated alone;
(h) ceritinib and afatinib co-encapsulated;
(i) osimertinib or afatinib alone;
(j) osimertinib and afatinib co-encapsulated;
(k) osimertinib or crizotinib alone;
(l) osimertinib and crizotinib co-encapsulated;
(m) dasatinib or ceritinib encapsulated alone;
(n) dasatinib and ceritinib co-encapsulated;
(o) afatinib or crizotinib alone;
(p) afatinib and crizotinib co-encapsulated;
(q) afatinib and nintedanib in about 30:1 to about 1:30 molar ratio;
(r) abemaciclib and sunitinib in about 30:1 to about 1:30 molar ratio
(s) dasatinib and afatinib in about 30:1 to about 1:30 molar ratio;
(t) ceritinib and afatinib in about 30:1 to about 1:30 molar ratio;
(u) osimertinib and afatinib in about 30:1 to about 1:30 molar ratio;
(v) osimertinib and crizotinib in about 30:1 to about 1:30 molar ratio;
(w) ceritinib and dasatinib in about 30:1 to about 1:30 molar ratio; and
(x) afatinib and crizotinib in about 30:1 to about 1:30 molar ratio.

In another embodiment, in the pharmaceutical composition, the combined kinase inhibitors can be released sequentially in a synergistic mode upon administration.

In another embodiment, in the pharmaceutical composition, the molar ratio of the co-encapsulated agents (e.g. the protein kinase inhibitors) is such that when the ratio is provided to cancer cells relevant to the cancer in an in-vitro assay over the concentration range at which the fraction of affected cells is about 0.20 to 0.80, synergy is exhibited over at least 20% of the range.

In another embodiment, in the pharmaceutical composition, the liposome encapsulated with the combined protein kinase inhibitors, maintains for at least one hour of the synergistic molar drug ratio in blood after in-vivo administration.

In other embodiment, the transmembrane pH gradient is formed by the concentration gradient of the ammonium ions, or the concentration gradient of the organic compound having an ammonium derivative or substituted ammonium ions.

In another aspect, the present disclosure provides a method of preparing a liposomal pharmaceutical composition by the active drug loading process using a transmembrane pH gradient or transition metals as the driving force, wherein the liposome is made by a process comprising the steps of:
(a) forming multilamellar liposome vesicles in a solution comprising water, lipid(s), and trapping agent(s);
(b) extruding the multilamellar liposome vesicles multiple times at an elevated temperature (e.g., in the range of 40-75° C.) through polycarbonate membranes (e.g., with a size of 50 nm or 100 nm) to form unilamellar liposomes;
(c) substantially removing the trapping agent(s) that are outside of the liposomes by diafiltration or size exclusion chromatography, or other buffer exchanging methods;
(d) heating the unloaded liposomes at an elevated temperature (e.g., 40-75° C.) in an aqueous solution comprising one or more active pharmaceutical ingredients (APIs, e.g., hydrophilic and water-soluble kinase inhibitors), thereby forming drug encapsulated liposomes; and
(e) adjusting the pH of the composition to about 5-8; and
(f) optionally, forming dry form of the product by lyophilization.

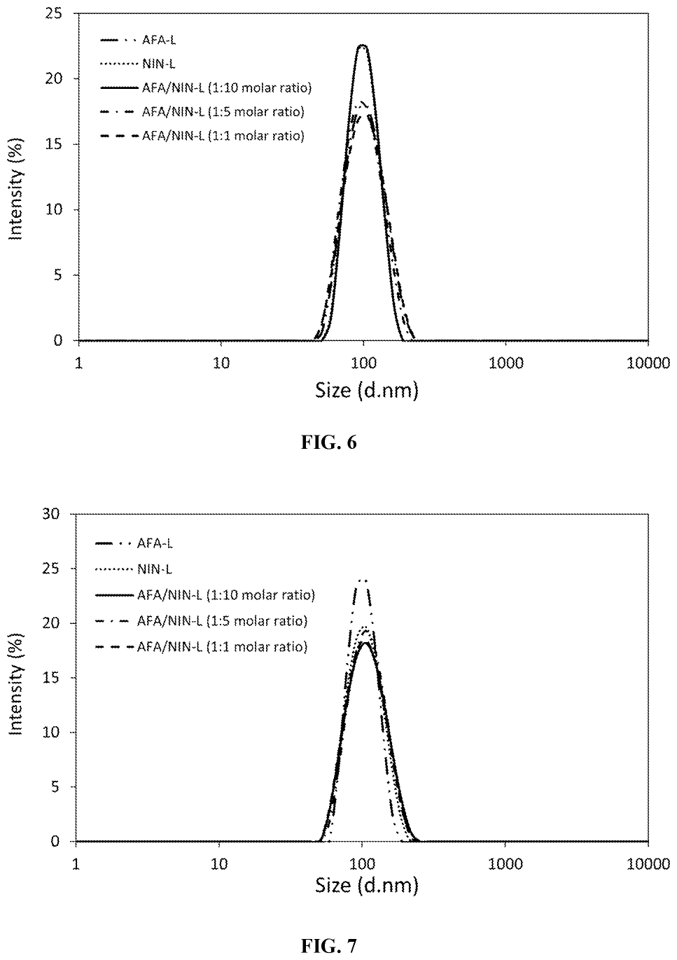

The architecture of the drug-loaded liposome prepared by the above-mentioned active drug loading method is illustrated in FIG. 5 (I).

In another aspect, the present disclosure provides a method of preparing a liposomal pharmaceutical composition with passive loading thereof; wherein the passive loading method is comprising the steps of:
(a) forming a lipid solution comprising at least one or more APIs (e.g., lipophilic and poorly water-soluble protein kinase inhibitors) in one type of, or a mixture of, organic solvent(s);
(b) evaporating the organic solvent(s) and hydrating the lipid/API mixture in aqueous solution to form liposome vesicles;
(c) reducing particle size by extruding, sonicating or homogenizing the lipid dispersion at an elevated temperature (e.g., 40-75° C.) to form unilamellar liposomes;
(d) adjusting the pH of the composition to about 5-8; and
(e) optionally, forming dry form of the product by lyophilization.

The architecture of the drug-loaded liposome prepared by the above-mentioned passive drug loading method is illustrated in FIG. 5 (III).

In another aspect, the present disclosure provides a method of preparing a liposomal pharmaceutical composition by sequentially performing passive loading first, and then active loading thereof; wherein the coupled passive/active loading is comprising the steps of:
(a) forming a lipid solution comprising at least one lipophilic protein kinase inhibitor in one type of, or a mixture of, organic solvent(s);
(b) evaporating the organic solvent(s) and hydrating the lipid/drug mixture in aqueous solution containing at least one type of trapping agent to form multilamellar liposomes;
(c) reducing particle size by extruding, sonicating or homogenizing the lipid dispersion at an elevated temperature (e.g., 40-75° C.) to form unilamellar liposomes;
(d) removing extra liposomal lipophilic protein kinase inhibitor(s) and the trapping agent(s) introduced from step (a) and (b) by diafiltration, size exclusion chromatography or other methods;
(e) heating the liposomes at an elevated temperature (e.g., 40-75° C.) in an aqueous solution comprising one or more hydrophilic protein kinase inhibitor(s) for drug loading;
(f) adjusting the pH of the composition to about 5-8; and
(g) optionally, forming dry form of the product by lyophilization.

The architecture of the drug-loaded liposome prepared by the above-mentioned coupled passive/active drug loading method is illustrated in FIG. 5 (II).

In some embodiments, the API is a protein kinase inhibitor or a combination of the protein kinase inhibitors.

In another aspect, the present disclosure provides a pharmaceutical composition according to any embodiments disclosed herein for use in the treatment of a cancer, cancer drug resistance and side effects in a subject in need of treatment, wherein the cancer is optionally selected from breast cancer, melanoma, gastrointestinal cancer, lung cancer, colorectal cancer, Ewing sarcoma, pancreatic cancer, prostate cancer, bladder cancer, kidney cancer, thyroid cancer, uterine cancer, and gastrointestinal stromal tumors, etc. Wherein the co-encapsulated kinase inhibitors can be delivered by the liposome with a synergistic cytotoxic or cytostatic effect on cancer cells.

In another aspect, the present disclosure provides a method of treating a cancer, comprising administering to a subject in need of treatment a therapeutically effective amount of a pharmaceutical composition according to any embodiments disclosed herein.

In some embodiments, the method of treatment using the liposomal pharmaceutical composition has reduced drug side effects as compared to administration of the kinase inhibitors in free form(s), e.g., tablet, capsule, injectable without liposomes, or the like.

In one embodiment, the cancer is non-small cell lung cancer.

In another embodiment, the cancer is colorectal cancer or renal cell cancer.

In another embodiment, the cancer is a colorectal or lung cancer, wherein the lung cancer is caused by either a high level of phosphorylation of a wild-type EGFR or a mutation within an EGFR amino acid sequence.

In another embodiment, the cancer is a colorectal or lung cancer, wherein the lung cancer is caused by VEGFA or a mutation within a VEGFA amino acid sequence.

In another embodiment, the treatment is a drug-resistant related cancer.

In one embodiment, the subject is a human.

In another embodiment, the subject is a non-human mammal or avian.

In some embodiments of the disclosure, the above-described lipid-based delivery vehicles comprise a third or fourth agent. Any therapeutic, diagnostic, or cosmetic agent may be included.

In another aspect, the present disclosure provides a plurality of drug-loaded liposomes as disclosed in any embodiment examples herein and/or prepared by a method according to any embodiments or examples disclosed herein.

In another aspect, the present disclosure provides a treatment kit comprising a container and a plurality of the drug-loaded liposomes according to any embodiments disclosed herein in the container, wherein the drug-loaded liposomes are or can be suspended in a sterile diluent solution ready for administration to a subject in need of treatment.

The lipid-based delivery vehicles of the present disclosure may be used not only in parenteral administration, but also in topical, nasal, subcutaneous, intraperitoneal, intramuscular, aerosol or oral delivery or by the application of the delivery vehicle onto or into a natural or synthetic implantable device at or near the target site for therapeutic purposes or medical imaging and the like. In one embodiment, the lipid-based delivery vehicles of the disclosure are used in parenteral administration, sometimes preferably, intravenous administration.

As those skilled in the art would understand, all the drug-loaded liposomes and pharmaceutical compositions prepared therefrom must be made under sterile conditions required of any materials and processes used in the administration to a subject in need of treatment. Thus, while the disclosure is not so limited, all the liposomes and pharmaceutical compositions disclosed or claimed herein and intended for treatment of a subject are sterile.

Some preferred embodiments herein described are not intended to be exhaustive or to limit the scope of the invention to the precise forms disclosed. They are chosen and described to explain the principles of the invention and its application and practical use to allow others skilled in the art to comprehend its teachings.

1. Composition

A. Therapeutic Agents: Protein Kinase Inhibitor

i. Tyrosine Kinase Inhibitors (TKIs)

Compounds that inhibit the tyrosine kinase pathway may be useful. Some preferred compounds are described in the studies are listed below.
ALK Drug Target: Avapritinib, Alectinib, Brigatinib, Crizotinib, Ceritinib, Lorlatinib, and the like;
BCR-Abl Drug Target: Bosutinib, Dasatinib, Imatinib, Nilotinib, Ponatinib, and the like;
BTK Drug Target: Ibrutinib, Acalabrutinib, Zanubrutinib, and the like;
c-Met Drug Target: Crizotinib, Cabozantinib, Capmatinib, and the like;
EGFR family Drug Target: Afatinib, Dacomitinib, Gefitinib, Erlotinib, Icotinib, Lapatinib, Neratinib, Vandetanib, Osimertinib, Tucatinib, and the like;
JAK family Drug Target: Baricitinib, Fedratinib, Ruxolitinib, Tofacitinib, and the like;
PDGFR α/β Drug Target: Axitinib, Avapritinib, Gefitinib, Imatinib, Lenvatinib, Nintedanib, Pazopanib, Regorafenib, Ripretinib, Sorafenib, Sunitinib, Upadacitinib, and the like;
RET Drug Target: Alectinib, Cabozantinib, Pralsetinib, Selpercatinib, Vandetanib, and the like;
Src Family Drug Target: Bosutinib, Dasatinib, Ponatinib, Vandetanib, and the like;
VEGFR Family Drug Target: Axitinib, Cabozantinib, Lenvatinib, Nintedanib, Regorafenib, Pazopanib, Sorafenib, Sunitinib, Vandetanib, and the like.

For example, afatinib is an oral irreversible ErbB family blocker that inhibits signaling from all EGFR-family of tyrosine kinase receptors EGFR (erbB1/HER1), HER 2 (erb2), and HER 4 (erb4). This small molecule compound was developed with aim of delaying acquired resistance to improving clinical outcomes versus first-generation EGFR inhibitor. Indeed, across a range of therapeutic areas and indications, afatinib monotherapy has demonstrated durable clinical activity that appears to compare favorably with other targeted therapies. Based on the significant improvements in PFS versus standard platinum-based chemotherapy in the pivotal Phase III studies, the oral afatinib tablet was approved in the USA, EU and Japan for the treatment of patients with non-small cell lung cancer (NSCLC) harboring distinct types of EGFR mutations in 2013. However, in all clinical studies, afatinib had a well-defined safety profile with predominantly gastrointestinal and cutaneous adverse events (AEs). The most frequent treatment-related grade ≥3 AEs with afatinib were diarrhea (5.4-14.4%), rash/acne (9.7-16.2%), and stomatitis/mucositis (5.4-8.7%) (Solca F, et al. (2012), J Pharmacol Exp Ther 343: 342-350).

Nintedanib, is a small molecule inhibitor that was approved for second-line treatment after chemotherapy failure combined with the cytotoxic docetaxel in patients with advanced lung adenocarcinoma. Nintedanib competitively binds to the ATP-binding sites within the kinase domains of VEGFR receptors (VEGFR) 1-3, PDGFR α/β, FGFR 1-4, and inhibits Src family tyrosine kinases (Src, Lck, Lyn), Flt-3, and RET. In addition, nintedanib exhibits further anticancer effects by reducing tumor growth and metastasis and was approved to treat patients with idiopathic pulmonary fibrosis (IPF). The oral nintedanib (capsule) was approved in EU in combination with docetaxel for second-line treatment of patients with advanced NSCLC of adenocarcinoma histology (2015), and in USA and EU for the treatment of patients with idiopathic pulmonary fibrosis (2014). However, despite the specific targeting of oncogene-dependent cells, the occurrence of severe side effects and rapid development of drug resistance, comparable to classic chemotherapy, are the major limitations for the successful treatment with kinase inhibitors in clinical studies (Hilberg F, et al., Cancer Res., 2008, 68: 4774-4782).

Another preferred TKI inhibitor, sunitinib (SUN), is a multi-targeted tyrosine kinase inhibitor that inhibits PDGFR (A and B), VEGFR1, VEGFR2, FLT3R, c-Kit, and RET-mediated signaling. In 2017, FDA approved sunitinib malate as SUTENT® by Pfizer as an adjuvant therapy for the treatment of adult patients with high risk of recurrent renal cell carcinoma (RCC) following nephrectomy, gastrointestinal stromal tumor (GIST) after disease progression on or intolerance to imatinib mesylate, advanced renal cell carcinoma. Progressive, well-differentiated pancreatic neuroendocrine tumors (pNET) in patients with unresectable locally advanced or metastatic disease.

ii. Other Kinase Inhibitors

Other kinase inhibitors for targeting cancer treatment may be useful. Some preferred compounds described in the studies are listed below.
B-Raf Serine/threonine Drug Target: Dabrafenib, Encorafenib, Vemurafenib, and the like;
CDK family Drug Target: Abemaciclib, Palbociclib, Sorafenib, Ribociclib, and the like;
CSF1R Drug Target: Pexidartinib, and the like;
FGFR1/2/3/4 Drug Target: Erdafitinib, Pemigatinib, and the like;
FKBP12/mTOR Drug Target: Everolimus, Sirolimus, Temsirolimus, and the like;
Flt3 Drug Target: Gilteritinib, Midostaurin, Quizartinib, and the like;
MEK1/2 Drug Target: Binimetinib, Cobimetinib, Selumetinib, Trametinib, and the like;
ROCK1/2 Drug Target: Netarsudil, and the like;
ROS1Drug Target: Entrectinib, and the like;
Syk Drug Target: Fostamatinib, and the like;
STK1 Drug Target: Quizartinib, and the like;
TRKA/B/C Drug Target: Entrectinib, Larotrectinib, and the like.

B. Parenteral Formulations

The compounds described herein can be formulated for parenteral administration. “Parenteral administration”, as used herein, means administration by any method other than through the digestive tract or non-invasive topical or regional routes. For example, parenteral administration may include administration to a patient intravenously, intradermally, intraarterially, intraperitoneally, intralesionally, intracranially, intraarticularly, intraprostatically, intrapleurally, intratracheally, intravitreally, intratumorally, intramuscularly, subcutaneously, subconjunctivally, intravesicularlly, intrapericardially, intraumbilically, by injection, and by infusion.

Parenteral formulations can be prepared as aqueous compositions using techniques known in the art. Typically, such compositions can be prepared as injectable formulations, for example, solutions or suspensions; solid forms suitable for use to prepare solutions or suspensions upon the addition of a reconstitution medium prior to injection; emulsions, such as water-in-oil (w/o) emulsions, oil-in-water (o/w) emulsions, and microemulsions thereof, liposome, or emulsomes.

The carrier can be a solvent or dispersion medium containing, for example, water, buffer, and isotonic agents, for example, sugars, HEPES buffer, or sodium chloride, etc.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

The formulation can contain a preservative to prevent the growth of microorganisms. Suitable preservatives include, but are not limited to, parabens, chloro-butanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, HEPES buffers, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the methods of preparation include vacuum-drying and freeze-drying techniques, which yield a powder of the active ingredient plus any additional desired ingredients from a previously sterile-filtered solution thereof. The powders can be prepared in such a manner that the particles are porous in nature, which can increase dissolution of the particles. Methods for making porous particles are well known in the art.

2. Controlled Release Formulations

The parenteral formulations described herein can be formulated for controlled release including immediate release, delayed release, extended release, pulsatile release, and combinations thereof.

For parenteral administration, the one or more compounds, and optionally one or more additional active agents, can be incorporated into microparticles, nanoparticles, or combinations thereof that provide controlled release of the compounds and/or one or more additional active agents. In embodiments wherein the formulations contain two or more drugs, the drugs can be formulated for the same type of controlled release (e.g., delayed, extended, immediate, or pulsatile) or the drugs can be independently formulated for different types of release (e.g., immediate and delayed, immediate and extended, delayed and extended, delayed and pulsatile, etc.).

For example, the compounds and/or one or more additional active agents can be incorporated into nano- and microparticles which provide controlled release of the drug(s). Release of the drug(s) is controlled by diffusion of the drug(s) out of the nano- and microparticles and/or degradation of the polymer particles by hydrolysis and/or enzymatic degradation. Suitable polymers include lipids and other natural or synthetic lipid derivatives.

Proteins which are water insoluble, such as zein, can also be used as materials for the formation of drug containing nano- and microparticles. Additionally, proteins, polysaccharides and combinations thereof which are water soluble can be formulated with drug into microparticles and subsequently cross-linked to form an insoluble network. For example, cyclodextrins can be complexed with individual drug molecules and subsequently cross-linked.

Encapsulation or incorporation of drug into carrier materials to produce drug containing nano- and microparticles can be achieved through known pharmaceutical formulation techniques. In the case of formulation in phospholipid or phospholipid-like materials, the carrier material is typically heated above its melting temperature and the drug is added to form a mixture comprising drug particles suspended in the carrier material, drug dissolved in the carrier material, or a mixture thereof. Nano- and microparticles can be subsequently formulated through several methods including, but not limited to, the processes of congealing, extrusion, spray chilling or aqueous dispersion. In some preferred processes, lipid is heated above its melting temperature, rehydrated in aqueous solution, extruded, and drug loaded. These processes are known in the art.

3. Determining In-Vitro Non-Antagonistic Combined Drug Ratios

In a further embodiment of the invention, the protein kinase inhibitors can be encapsulated into liposomes at synergistic or additive (i.e., non-antagonistic) ratios. The therapeutically effective non-antagonistic ratio of the protein kinase inhibitors is determined by assessing the biological activity or effects of the agents on relevant cell culture and/or tumor homogenates from individual patient biopsies, over a range of concentrations. Any method which results in determination of a ratio of the protein kinase inhibitor agents which maintains a desired therapeutic effect may be used. For example, unless otherwise noted, the Chou-Talalay median-effect method was used in the examples disclosed in this application (Chou, T. C., J. Theor. Biol., 1976, 39:253-276).

The underlying experimental data are generally determined in-vitro using cells in culture. Sometimes preferably, the combination index (CI), which is plotted as a function of the fraction of cells affected (Fa), as explained above, is a surrogate parameter for concentration range. Preferred combinations of agents are those that display synergy or additivity over a substantial range of Fa values. Combinations of agents are selected if non-antagonistic over at least about 5% of the concentration range wherein greater than 1% of the cells are affected, i.e., a Fa range greater than 0.01. Sometime preferably, a larger portion of overall concentration exhibits a favorable CI; for example, 5% of a Fa range of 0.2-1.0. Sometimes more preferably about 10% of this range exhibits a favorable CI. Sometimes even more preferably, about 20% of the Fa range, over about 50%, or over at least about 70% of the Fa range of 0.2 to 1.0 are utilized in the compositions. Combinations that display synergy over a substantial range of Fa values may be re-evaluated at a variety of agent ratios to define the optimal ratio to enhance the strength of the non-antagonistic interaction and increase the Fa range over which synergy is observed.

While it would be desirable to have synergy over the entire range of concentrations over which cells are affected, it has been observed that in many instances, the results are considerably more reliable in a Fa range of 0.2-0.8 when using a spectrophotometric method such as the MTT assay. Thus, although the synergy exhibited by combinations of the invention is set forth to exist within the broad range of 0.01 or greater, sometimes preferably the synergy is established in the Fa range of 0.2-0.8. Other more sensitive assays, however, can be used to evaluate synergy at Fa values greater than 0.8, for example, bioluminescence or clonogenicity assays.

The optimal combination ratio may be further used as a single pharmaceutical unit to determine synergistic or additive interactions with a third agent. In addition, a three-agent combination may be used as a unit to determine non-antagonistic interactions with a fourth agent, and so on.

As set forth above, the in-vitro studies on cell cultures will be conducted with “relevant” cells. The choice of cells will depend on the intended therapeutic use of the agent. Only one relevant cell line or cell culture type needs exhibit the required non-antagonistic effect in order to provide a basis for the compositions to come within the scope of the invention.

For example, in one preferred embodiment of the invention, the combination of agents is intended for anticancer therapy. In a frequent embodiment, the combination of agents is intended for multiple cancers, such as leukemia or lymphoma therapy, breast cancer, triple negative breast cancer, gastrointestinal cancer, colorectal cancer, RCC, and lung cancer. Appropriate choices will then be made of the cells to be tested and the nature of the test. In particular, tumor cell lines are suitable subjects and measurement of cell death or cell stasis is an appropriate end point. As will further be discussed below, in the context of attempting to find suitable non-antagonistic combinations for other indications, other target cells and criteria other than cytotoxicity or cell stasis could be employed.

For determinations involving antitumor agents, cell lines may be obtained from standard cell line repositories (NCI or ATCC for example), from academic institutions or other organizations including commercial sources. Some preferred cell lines would include one or more selected from cell lines identified by the Developmental Therapeutics Program of the NCI/NIH. The tumor cell line screen used by this program currently identifies about 60 different tumor cell lines representing leukemia, melanoma, and cancers of the lung, colon, brain, ovary, breast, prostate, stomach, and kidney, etc. The required non-antagonistic effect over a desired concentration range need be shown only on a single cell type; however, sometimes preferably at least two cell lines, sometimes more preferably three cell lines, five cell lines, or even 10 cell lines, exhibit this effect. The cell lines may be established tumor cell lines or primary cultures obtained from patient samples. The cell lines may be from any species, but the preferred source will be mammalian and in particular human. The cell lines may be genetically altered by selection under various laboratory conditions.

In one preferred embodiment, the given effect (Fa) refers to cell death or cell stasis after application of a cytotoxic agent to a cell culture. Cell death or viability may be measured by MTT assay in this invention. Non-antagonistic ratios of two or more agents can be determined for disease indications other than cancer and this information can be used to prepare therapeutic formulations of two or more drugs for the treatment of these diseases. With respect to in-vitro assays, many measurable endpoints can be selected from which to define drug synergy, provided those endpoints are therapeutically relevant for the specific disease. As set forth above, the in-vitro studies on cell cultures will be conducted with “relevant” cells. The choice of cells will depend on the intended therapeutic use of the agent. In-vitro studies on individual patient biopsies or whole tumors can be conducted with “tumor homogenate.” generated from homogenization of the tumor sample(s) into single cells. In one preferred embodiment, the given effect (Fa) refers to cell death or cell stasis after application of a cytotoxic agent to a “relevant” cell culture. Cell death or viability may be measured using a number of the methods known in the art.

4. Preparation of Lipid-Based Delivery Vehicles

Among the nanoparticle delivery systems, liposome is one of the most widely used pharmaceutical carriers with several unique characteristics including: (1) prolonged drug circulation half-life mediated by the carrier, (2) reduced nonspecific uptake, (3) increased accumulation at the tumor site through the passive enhanced permeation and retention (EPR) effect and/or active targeting by incorporation of targeting ligands, (4) predominantly endocytosis uptake with the potential to bypass mechanisms of multidrug resistance, and (5) ability to tailor the relative ratios of each agent based on its pharmacological disposition, (6) a single delivery system carrying multiple drugs (hydrophilic and lipophilic drugs) in the same platform can lead to synchronized and controlled pharmacokinetics of each drug, resulting in improved drug efficacy and (7) improved drug solubility and bioavailability (Mamot, C., et al., Drug Resist. Updates 2003, 6, 271-279).

Sometimes preferably, the lipid carriers for use in this invention are liposomes. Suitable liposomes for use in this invention include large unilamellar vesicles (LUVs), multilamellar vesicles (MLVs), small unilamellar vesicles (SUVs) and interdigitating fusion liposomes. Liposomes for use in this invention may be prepared to contain a phosphatidylcholine lipid or phospholipid-like material, such as distearylphosphatidylcholine (DSPC) or hydrogenated soy phosphatidylcholine (HSPC).

Liposomes of the invention may also contain a sterol, such as cholesterol. Liposomes may also contain therapeutic lipids, which examples include ether lipids, phosphatidic acid, phosphonates, ceramide and ceramide analogs, sphingosine and sphingosine analogs and serine-containing lipids.

Liposomes may also be prepared with surface stabilizing hydrophilic polymer-lipid conjugates, such as polyethylene glycol-DSPE, to enhance circulation longevity. The incorporation of negatively charged lipids such as phosphatidylglycerol (PG) and phosphatidylinositol (PI) may also be added to liposome formulations to increase the circulation longevity of the carrier. These lipids may be employed to replace hydrophilic polymer-lipid conjugates as surface stabilizing agents. Sometimes preferably, the liposomes may contain phosphatidylglycerol (PG) and/or phosphatidylinositol (PI) to prevent aggregation, thereby increasing the blood residence time of the carrier.

In one embodiment, liposome compositions in accordance with this invention are used to treat cancer and infection disease. Delivery of encapsulated drugs to a tumor site is achieved by administration of liposomes of the invention. Sometimes preferably liposomes have a mean diameter of particle size less than 300 nm. Sometimes more preferably liposomes have a mean diameter of particle size less than 200 nm. Tumor vasculature is generally leakier than normal vasculature due to fenestrations or gaps in the endothelia. This allows delivery vehicles of 200 nm or less (average diameter) to penetrate the discontinuous endothelial cell layer and underlying basement membrane surrounding the vessels supplying blood to a tumor. Selective accumulation of the delivery vehicles into tumor sites following extravasation leads to enhanced anticancer drug delivery and therapeutic effectiveness.

Various methods may be utilized to encapsulate active agents in liposomes. “Encapsulation” includes covalent or non-covalent association of an agent with the lipid-based delivery vehicle. For example, this can be by interaction of the agent with the outer layer or layers of the liposome or entrapment of an agent within the liposome, equilibrium being achieved between different portions of the liposome. Thus, encapsulation of an agent can be by association of the agent by interaction with the bilayer of the liposomes through covalent or non-covalent interaction with the lipid components or entrapment in the aqueous interior of the liposome, or in equilibrium between the internal aqueous phase and the bilayer. “Loading” refers to the act of encapsulating one or more agents into a delivery vehicle.

Encapsulation of the desired combination can be achieved either through encapsulation in separate delivery vehicles or within the same delivery vehicle. Where encapsulation into separate liposomes is desired, the lipid composition of each liposome may be quite different to allow for coordinated pharmacokinetics. By altering the liposome vehicle composition, release rates of encapsulated drugs can be matched to allow desired ratios of the drugs to be delivered to the tumor site. Means of altering release rates include increasing the acyl chain length of vesicle forming lipids to improve drug retention, controlling the exchange of surface grafted hydrophilic polymers such as polyethylene glycol group on mPEG-DSPE out of the liposome membrane, and incorporating membrane-rigidifying agents such as sterols or sphingomyelin into the membrane. It should be apparent to those skilled in the art that if a first and second drug are desired to be administered at a specific drug ratio and if the second drug is retained poorly within the liposome composition of the first drug (e.g., DMPC/Chol), that improved pharmacokinetics may be achieved by encapsulating the second drug in a liposome composition with lipids of increased acyl chain length (e.g., DSPC/Chol). When encapsulated in separate liposomes, it should be readily accepted that ratios of both drugs that have been determined on a patient-specific basis to provide optimal therapeutic activity can be generated for individual patients by combining the appropriate amounts of each liposome encapsulated drug prior to administration. Alternatively, two or more agents may be encapsulated within the same liposome.

Techniques for encapsulation are dependent on the nature of the therapeutic agents and delivery vehicles. For example, therapeutic agents may be loaded into liposomes using both passive and active loading methods. Passive methods of encapsulating active agents in liposomes involve encapsulating the agents during the preparation of the liposomes. This technique results in the formation of multilamellar vesicles (MLVs) that can be converted to large unilamellar vesicles (LUVs) or small unilamellar vesicles (SUVs) upon extrusion. In addition, another suitable method of passive encapsulation involves passive equilibration after the formation of liposomes. This process involves incubating preformed liposomes under altered or non-ambient (based on temperature, pressure, etc.) conditions and adding a therapeutic agent (e.g., protein kinase inhibitors) to the exterior of the liposomes. The therapeutic agent then equilibrates into the interior of the liposomes by passing the liposomal membrane. The liposomes are then returned to ambient conditions and the unencapsulated therapeutic agent, if present, is removed via dialysis or another suitable method. Examples of drug-loaded liposomes prepared by the above-mentioned passive drug loading method is illustrated in FIG. 5 (III).

Active loading methods of drug encapsulation include the pH gradient loading approach and the active transition metal-loading technique. Loading method based on the transmembrane pH gradient utilizes ammonium or substituted ammonium salts of monoanions or polyanions as the trapping agent which is pre-loaded into the liposome prior to the encapsulation of the therapeutic agent. Those trapping agents establish the transmembrane pH gradient and also may form precipitation, aggregation, or gelation with the therapeutic agent, both of which serve as the driving force for the active loading of the agents into the liposome. Regarding the pH gradient, it is generally accepted that the pH value difference between the internal and external environment of the liposome is at least greater than one unit. Other methods employed to establish and maintain a pH gradient across a liposome involve the use of an ionophore that can insert into the liposome membrane and transportation across membranes in exchange for protons. Wherein, the active transition metal-based loading technique utilizes transition metals to drive the uptake of the agents into liposomes via complexation or coordination. Examples of drug-loaded liposomes prepared by the above-mentioned active drug loading method is illustrated in FIG. 5 (I).

Suitable trapping agents may be anionic, cationic, amphoteric, or nonionic active agents including, but are not limited to those containing carboxylate, polyphosphate, sulfonate including long chain alkyl sulfonates and alkyl aryl sulfonates and sulfate. Cationic trapping agents include quaternary ammonium compounds such as benzalkonium chloride, benzethonium chloride, cetrimonium bromide, stearyl dimethylbenzyl ammonium chloride, polyoxyethylene and coconut amine, and the like.

More specific examples of trapping agents include ammonium sulfate, transition metals and ammonium or substituted ammonium salts of the following: polyanionized sulfated cyclodextrin, sulfobutyl ether cyclodextrin, polyanionized sulfated sugar, polyphosphates, and the like.

Specifically, trapping agents include ammonium or substituted ammonium salts of the following polyanionized sulfated sugars: sucrose octasulfate, chondroitin sulfate, dermatan sulfate, keratan sulfate, heparan sulfate and sulfated hyaluronic acid, fucoidan, galactan, carrageenan, rhamnan sulfate, galactofucan, mannoglucuronofucan, arabinogalactans sulfate, mannan sulfate, sulfated heterorhamnan and xylomannan sulfate, and the like.

Specifically, trapping agents include ammonium or substituted ammonium salt of the following forms of sulfobutylether cyclodextrin: sulfobutylether-α-cyclodextrin, sulfobutylether-β-cyclodextrin, and sulfobutylether-γ-cyclodextrin, and the like.

Specifically, trapping agents include ammonium or substituted ammonium salts of the following polyphosphate: phytic acid, triphosphoric acid, polyphosphoric acid and cyclic trimetaphosphate.

Specifically, the counter ion to the above polyanions includes ammonium and substituted ammonium which further includes the protonated form of the following: triethylamine, triethanolamine, tris(hydroxymethyl)aminomethane or tromethamine, diethanolamine, ethylenediamine, tributylamine, 1,4-diazabicyclo[2.2.2]octane, diethylethanolamine, diethanolethylamine, ethanolamine, morpholine, and the like.

Transition metal ions-based trapping agents include the salt form of the following: ions of copper, zinc, manganese, nickel, and cobalt. The counter ion to the metal includes sulfate, chloride, gluconate, bromide, and hydroxide.

More specifically, trapping agents used for drug loading in liposome include the following: ammonium sulfate, triethylammonium sucrose octasulfate (TEA-SOS), triethylammonium sulfobutyl ether beta-cyclodextrin (TEA-SBE-β-CD); tris(hydroxymethyl)aminomethane salt of sulfobutyl ether-beta-cyclodextrin (Tris-SBE-β-CD), triethylammonium salt of phytic acid or inositol hexaphosphate (TEA-IP6), copper gluconate, copper sulfate, copper chloride and zinc sulfate.

Passive and active drug loading methods of entrapment may also be coupled in order to prepare a liposome formulation containing both lipophilic and hydrophilic drugs into a single delivery vehicle. Specifically, lipophilic drugs can be loaded into the liposome by passive loading first, then the same liposome is subsequently used to load hydrophilic drugs via the active loading approach. Examples of drug-loaded liposomes prepared by the above-mentioned coupled passive/active drug loading method is illustrated in FIG. 5 (II).

5. Administering Compositions of the Invention In-Vivo

As mentioned above, the delivery vehicle compositions of the present invention may be administered to warm-blooded animals, including humans as well as to domestic avian species. For treatment of human ailments, a qualified physician will determine how the compositions of the present invention should be utilized with respect to dose, schedule and route of administration using established protocols. Such applications may also utilize dose escalation should agents encapsulated in delivery vehicle compositions of the present invention exhibit reduced toxicity to healthy tissues of the subject.

In one embodiment, the pharmaceutical compositions of the present invention are administered parenterally, i.e., intraarterially, intravenously, intraperitoneally, subcutaneously, or intramuscularly. Sometimes preferably, the pharmaceutical compositions are administered intravenously or intraperitoneally by a bolus or infusion injection.

In other methods, the pharmaceutical or cosmetic preparations of the present invention can be contacted with the target tissue by direct application of the preparation to the tissue. The application may be made by topical, “open” or “closed” procedures. By “topical”, it is meant the direct application of the multi-drug preparation to a tissue exposed to the environment, such as the skin, oropharynx, external auditory canal, and the like. “Open” procedures are those procedures that include incising the skin of a patient and directly visualizing the underlying tissue to which the pharmaceutical preparations are applied. This is generally accomplished by a surgical procedure, such as a thoracotomy to access the lungs, abdominal laparotomy to access abdominal viscera, or other direct surgical approach to the target tissue. “Closed’ procedures are invasive procedures in which the internal target tissues are not directly visualized, but accessed via inserting instruments through small wounds in the skin. For example, the preparations may be administered to the peritoneum by needle lavage. Alternatively, the preparations may be administered through endoscopic devices.

Pharmaceutical compositions comprising delivery vehicles of the invention are prepared according to standard techniques and may comprise water, buffer, 0.9% saline, 0.3% glycine, 5% dextrose, iso-osmotic sucrose solutions and the like, including glycoproteins for enhanced stability, such as albumin, lipoprotein, globulin, and the like. These compositions may be sterilized by conventional, well known sterilization techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and the like. Additionally, the delivery vehicle suspension may include lipid-protective agents which protect lipids against free-radical and lipid peroxidative damages on storage. Lipophilic free-radical quenchers, such as alpha-tocopherol and water-soluble iron specific chelators, such as ferrioxamine, are suitable.

The concentration of delivery vehicles in the pharmaceutical formulations can vary widely, such as from less than about 0.05%, usually at or at least about 2-5% to as much as 10 to 30% by weight and will be selected primarily by fluid volumes, viscosities, and the like, in accordance with the particular mode of administration selected. For example, the concentration may be increased to lower the fluid load associated with treatment. Alternatively, delivery vehicles composed of irritating lipids may be diluted to low concentrations to lessen inflammation at the site of administration. For diagnosis, the amount of delivery vehicles administered will depend upon the particular label used, the disease state being diagnosed and the judgment of the clinician.

Dosage for the delivery vehicle formulations will depend on the ratio of drug to lipid and the administrating physician's opinion based on age, weight, and condition of the patients.

In addition to pharmaceutical compositions, suitable formulations for veterinary use may be prepared and administered in a manner suitable to the subjects. Preferred veterinary subjects include mammalian species, for example, non-human primates, dogs, cats, cattle, horses, sheep, and domesticated fowl. Subjects may also include laboratory animals, for example, in particular, rats, rabbits, mice, and guinea pigs.

6. Package Kit

The therapeutic agents in the inventive compositions may be formulated separately in individual compositions wherein each therapeutic agent is stably associated with appropriate delivery vehicles. These compositions can be administered separately to subjects as long as the pharmacokinetics of the delivery vehicles are coordinated so that the ratio of therapeutic agents administered is maintained at the target for treatment. Thus, it is useful to construct kits which include, in separate containers, a first composition comprising delivery vehicles stably associated with at least a first therapeutic agent and, in a second container, a second composition comprising delivery vehicles stably associated with at least one second therapeutic agent. The containers can then be packaged into the package kit.

The kit will also include instructions as to the mode of administration of the compositions to a subject, at least including a description of the ratio of amounts of each composition to be administered. Alternatively, or in addition, the kit is constructed so that the amounts of compositions in each container is pre-measured so that the contents of one container in combination with the contents of the other represent the correct ratio. Alternatively, or in addition, the containers may be marked with a measuring scale permitting dispensation of appropriate amounts according to the scales visible. The containers may themselves be useable in administration; for example, the kit might contain the appropriate amounts of each composition in separate syringes. Formulations which comprise the pre-formulated correct ratio of therapeutic agents may also be packaged in this way so that the formulation is administered directly from a syringe prepackaged in the kit.

### Definitions

Unless defined otherwise, all terms of art, notations and other scientific terms or terminology used herein have the same meaning as is commonly understood by one of ordinary skill in the art to which this invention belongs. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a,” “an,” and “the” include plural reference, and vice versa, any plural forms include singular reference, unless the context clearly dictates otherwise.

The term “about” or “approximately”, unless otherwise defined, generally includes up to plus or minus 10% of the indicated number. For example, “about 10%” may indicate a range of 9% to 11%, and “about 20” may mean from 18 to 22. Sometimes preferably, “about” includes up to plus or minus 5% of the indicated value. Alternatively, “about” includes up to plus or minus 5% of the indicated value. When “about” is used before a range, it is applicable to both the lower end and the upper end of a range.

The term “substantially” as herein used means “for the most part” or “essentially,” as would be understood by a person of ordinary skill in the art, and if measurable quantitatively, refers to at least 90%, preferably at least 95%, more preferably at least 98%.

The terms “comprising”, “having”, “including”, and “containing”, or the like, are to be construed as open-ended terms (i.e., meaning “including, but not limited to”) unless otherwise noted.

As used herein, the term “synergistic effect” means an interaction between two or more drugs that causes the total effect of the drugs to be greater than the sum of the individual effects of each drug.

By “synergistic ratio” is meant the molar ratio of two or more drugs used in combination at which a synergistic effect can be obtained.

As used herein, the term “synergistic cytotoxic effect” refers an interaction between two or more drugs that causes the total effect of the drugs to be greater than the sum of the individual effects of each drug. This total effect results in cell kill and eventual tumor shrinkage.

As used herein, the term “synergistic cytostatic effect” refers an interaction between two or more drugs that causes the total effect of the drugs to be greater than the sum of the individual effects of each drug. This total effect results in tumor growth inhibition without direct cell killing.

The term “additive effect” means the combined effect produced by the action of two or more drugs, being equal to the sum of their separate effects.

By “additive ratio” is meant the molar ratio of the two or more drugs used in combination at which an additive effect can be obtained.

The term “non-antagonistic ratio” refers to both synergistic and additive ratio.

As used herein, the term “antagonistic effect” means a therapeutic response to exposure to two or more drugs that is less than would be expected if the known effects of the individual drugs were added together.

The term “antagonistic ratio” as used herein refers to molar ratio of two or more drugs used in combination at which an antagonistic effect can be obtained.

The term “combination index” refers to a parameter that is used to determine the degree of drug interaction. Combination Index (CI) can be calculated based on the median-effect analysis algorithm as described by Chou and Talalay (T. C. Chou and P. Talalay, Adv. Enzyme Reg., 1984, 22:27-55). A CI value <0.9 indicates synergistic drug interactions; 0.9≤CI≤1.1 reflects additive effect, and a CI >1.1 indicates antagonistic effect.

The term “fraction affected” refers to the faction of cells that is affected by a particular drug dose on their growth in an in vitro assay. Fraction affected is used to calculated combination index as described by Chou and Talalay procedure.

By “relevant” cells refer to at least one cell culture or cell line which is appropriate for testing the desired biological effect. As these agents are used as antineoplastic agents, “relevant” cells are those of cell lines identified by the Developmental Therapeutics Program (DTP) of the National Cancer Institute (NCI)/National Institutes of Health (NIH) as useful in their anticancer drug discovery program. Currently the DTP screen utilizes 60 different human tumor cell lines. The desired activity on at least one of such cell lines would need to be demonstrated.

By “tumor homogenate” refers to cells generated from the homogenization of patient biopsies or tumors. Extraction of whole tumors or tumor biopsies can be achieved through standard medical techniques by a qualified physician and homogenization of the tissue into single cells can be carried out in the laboratory using a number of methods well-known in the art.

The term “trapping agent” as used herein refers to a chemical compound that is presented within the aqueous compartment of the liposome and is used to entrap and retain one or more drugs within the same location inside of the liposome.

The term “liposome” refers to a spherical-shaped vesicle that is composed of one or more phospholipid bilayers, which closely resembles the structure of cell membranes.

The phrase “unilamellar vesicles” as used herein refers to spherical vesicles comprised of one lipid bilayer membrane which defines a single closed aqueous compartment. The bilayer membrane is composed of two layers of lipids: an inner layer and an outer layer. Lipid molecules in the outer layer are oriented with their hydrophilic head portions towards the external aqueous environment and their hydrophobic tails pointed downward toward the interior of the liposome. The inner layer of the lipid lays directly beneath the outer layer, the lipids are oriented with their heads facing the aqueous interior of the liposome and their tails towards the tails of the outer layer of lipid.

The phrase “multilamellar vesicles” as used herein refers to liposomes that are composed of more than one lipid bilayer membrane, which membranes define more than one closed aqueous compartment. The membranes are concentrically arranged so that the different membranes are separated by aqueous compartments, much like an onion.

By “protein kinase inhibitors” is meant a large group of unique and potent antineoplastic agents which specifically target protein kinases that are altered in cancer cells and that account for some of their abnormal growth. The effect of protein kinase inhibitors is usually cytostatic, which means tumor growth is inhibited without direct cell killing. Therefore, protein kinase inhibitors are less toxic and in the right patient population, protein kinase inhibitors are more potent than conventional chemotherapeutic agents.

By “release” is meant that the drug encapsulated in a liposome passes through the lipid membrane constituting the liposome and then exits to the outside of the liposome.

The term “encapsulation” as used herein, refers to encircling an internal phase typically resulting in an interior cavity separated from an external media. The components of the internal phase/interior cavity are thus “encapsulated” as described herein. As described herein, the encircled, or encapsulated, internal phase is the lipid bilayers and the aqueous phases. The amount of the therapeutic drug that is loaded into the interior cavity of the liposome and therefore unavailable to the external media until the liposome is triggered from release would be considered as “encapsulated” within the liposome.

The phrase “co-encapsulation” and “co-encapsulated” as used herein, refers to the situation where two or more therapeutic agents are encapsulated within the liposome.

The term “passive loading” as used herein, refers to a drug loading technique used in liposome drug product preparation. In one scenario, passive loading can be achieved by encapsulating the therapeutic agent during the liposome formation. In another scenario, passive loading involves passive drug equilibration after the formation of liposomes.

The phrase “active loading” as used herein refers to a drug loading technique used in liposome drug product preparation. The commonly used active loading methods in the art include the transmembrane pH gradient loading technique and transition metal loading technique. The former one utilizes an ammonium or a substituted ammonium salt of monoanion or polyanions as the trapping agent which is pre-loaded into the liposome prior to the encapsulation of therapeutic agent. Based on the equilibrium as determined by the pH gradient, the therapeutic agent can “actively” diffuse into the aqueous compartment of the liposome, interact with the pre-loaded trapping agent through the formation of precipitation, aggregation, or gelation, which serves as another driving force to encapsulate the therapeutic agent inside the liposome. The transition metal-based loading technique utilizes transition metals to drive the uptake of the agents into liposomes via complexation or coordination. Overall, a much higher encapsulation efficiency of the therapeutic agent can be achieved (e.g., >90%) by using the active loading technique as compared to that obtained from the passive loading technique.

The term “mean particle size” refers to the average diameter of the liposome. This can be measured by instrument based on dynamic light scattering.

The term “substituted ammonium” means that the hydrogen atoms in the ammonium ion are substituted with one or more alkyl group or some other organic group to form a substituted ammonium ion.

The term “triple negative breast cancer” refers to a type of breast cancer from which the cancer cells do not have estrogen or progesterone receptors, and also do not make enough of the protein called human epidermal growth factor receptor 2 (HER2). Namely, the cells test “negative” on all three tests of the above receptors.

The term “non-small cell lung cancer” (NSCLC) refers to any type of epithelial lung cancer other than small cell lung cancer (SCLC). The most common types of NSCLC are squamous cell carcinoma, large cell carcinoma, and adenocarcinoma, but there are several other types that occur less frequently, and all types can occur in unusual histologic variants.

The term “renal cell cancer” (RCC) refers to a type of kidney cancer that originates in the lining of the proximal convoluted tubule, a part of the very small tubes in the kidney that transport primary urine. RCC is the most common type of kidney cancer in adults, responsible for approximately 90-95% of cases.

The term “drug-resistant cancer” refers to the type of cancer that show resistance to the given therapeutic agents. Drug resistance occurs when cancer cells don't respond to a drug that is usually able to kill or weaken them. Drug resistance may be present before treatment is given (intrinsic resistance) or may occur during or after treatment with the drug (acquired resistance). In cancer treatment, there are many things that may cause resistance to anticancer drugs. For example, DNA changes or other genetic changes may change the way the drug gets into the cancer cells or the way the drug is broken down within the cancer cells. Drug resistance can lead to cancer treatment not working or to the cancer coming back.

The term “effective amount” refers to the amount necessary or sufficient to realize a desired biologic or therapeutic effect.

The term “therapeutically effective amount” means an amount effective to deliver a therapeutically effective amount of an amount of active agent needed to delay the onset of, inhibit the progression of, or halt altogether the particular disease, disorder or condition being treated, or to otherwise provide the desired effect on the subject to be treated. As one of ordinary skill in the art would understand, a therapeutically effective amount varies with the patient's age, condition, and gender, as well as the nature and extent of the disease, disorder or condition in the patient, and the dosage may be adjusted by the individual physician (or veterinarian).

The term “pharmaceutically acceptable” describes a material that is not biologically or otherwise undesirable, i.e., without causing an unacceptable level of undesirable biological effects or interacting in a deleterious manner.

The terms “treating” and “treatment”, or the like, refer to reversing, alleviating, inhibiting, or slowing the progress of the disease, disorder, or condition to which such terms apply, or one or more symptoms of such disease, disorder, or condition.

The term “subject” or “patient” used herein refers to a human patient or a mammalian animal, such as cat, dog, cow, horse, monkey, or the like.

The term “total lipid” refers to all the lipids and lipid derivatives used in the formulation, which include phospholipids (e.g., HSPC, DSPC, DPPC, DMPC and DSPG), sterol (e.g., cholesterol), and phospholipid conjugated with polyethylene glycol (e.g., mPEG-DSPE).

The present invention is further described by the following examples. The examples are provided solely to illustrate certain aspects of the invention by reference to specific embodiments. These exemplifications, while illustrating certain specific embodiments of the invention, do not portray the limitations or circumscribe the scope of the disclosed invention.

The following abbreviations are used in this application:
ABE: Abemaciclib
ABE-L: Liposome encapsulated with abemaciclib
ABE/SUN-L: Liposome co-encapsulated with abemaciclib and sunitinib
AE: Adverse event
AFA: Afatinib
AFA-L: Liposome encapsulated with afatinib
AFA/CER-L: Liposome co-encapsulated with afatinib and ceritinib
AFA/DAS-L: Liposome co-encapsulated with afatinib and dasatinib
AFA/NIN-L: Liposome co-encapsulated with afatinib and nintedanib
APIs: Active pharmaceutical ingredients
CER: Ceritinib
CER-L: Liposome encapsulated with ceritinib
Chol: Cholesterol
CI: Combination index
CRI Crizotinib
CRI-L: Liposome encapsulated with crizotinib
DAS: Dasatinib
DAS-L: Liposome encapsulated with dasatinib
DAS/CER-L: Liposome co-encapsulated with dasatinib and ceritinib
DDPC: 1,2-Didecanoyl-sn-glycero-3-phosphocholine
DEPC: 1,2-Dierucoyl-sn-glycero-3-phosphocholine
DLPC: 1,2-dilauroyl-sn-glycero-3-phosphocholine
DMPC: 1,2-dimyristoyl-sn-glycero-3-phosphocholine
DPPC: 1,2-dipalmitoyl-sn-glycero-3-phosphocholine
DSPC: 1,2-distearoyl-sn-glycero-3-phosphocholine
DSPG: 1,2-Distearoyl-sn-glycero-3-phosphoglycerol
EDTA: Ethylenediaminetetraacetic acid
ED₇₅ and ED₉₀: Effective dose required to affect 75 and 90% of the cells in cell culture
Fa: Fraction affected
GIST: Gastrointestinal stromal tumor
HBS: HEPES buffered saline (20 mM HEPES, 150 mM NaCl, pH 7.4)
HEPES: N-2-hydroxylethyl-piperazine-N-2-ethanesulfonic acid
HSPC: L-α-phosphatidylcholine, hydrogenated
LUV: large unilamellar vesicle
MLV: Multilamellar vesicle
mPEG-2000-DSPE, sodium salt: N-(Carbonyl-methoxypolyethylenglycol-2000)-1,2-distearoyl-sn-glycero-3-phosphoethanolamine sodium
MTT: 3-(4,5-dimethylthiazol-2-yl)-2,5-diphenyl-2-H tetrazolium bromide
NIN: Nintedanib
NIN-L: Liposome encapsulated with nintedanib
NSCLC: Non-small cell lung cancer
OSI: Osimertinib
OSI-L: Liposome co-encapsulated with osimertinib
OSI/AFA-L: Liposome co-encapsulated with osimertinib and afatinib
OSI/CRI-L: Liposome co-encapsulated with osimertinib and crizotinib
PG: Phosphatidylglycerol
PSPC: 1-palmitoyl-2-stearoyl-sn-glycero-3-phosphocholine
RCC: Renal cell carcinoma
SBE-α-CD: Sulfobutylether-α-cyclodextrin
SBE-β-CD: Sulfobutylether-β-cyclodextrin
SBE-γ-CD: Sulfobutylether-γ-cyclodextrin
SMPC: 1-stearoyl-2-myristoyl-sn-glycero-3-phosphocholine
SOPC: 1-stearoyl-2-oleoyl-sn-glycero-3-phosphocholine
SPPC: 1-stearoyl-2-palmitoyl-sn-glycero-3-phosphocholine
SUN: Sunitinib
SUN-L: Liposome encapsulated with sunitinib
SUV: Small unilamellar vesicle
TEA: Triethylamine
TEA-SOS: Triethylammonium sucrose octasulfate
TEA-SBE-β-CD: Triethylammonium sulfobutylether-β-cyclodextrin
Tris-SBE-β-CD: Tris(hydroxymethyl) aminomethane sulfobutylether-β-cyclodextrin

### Experimental Methods

### Materials

All the protein kinase inhibitors were purchased from Sigma-Aldrich Co. (St Louis, MO, USA), such as afatinib dimeleate (AFA), nintedanib esylate (NIN), abemaciclib mesylate (ABE), sunitinib malate (SUN), crizotinib (CRI), dasatinib monohydrate (DAS), ceritinib (CER), osimertinib mesylate (OSI), and others. Hydrogenated Soy Phosphatidylcholine (HSPC), 1,2-distearoyl-sn-glycero-3-phosphocholine (DSPC), Distearoylphosphatidylglycerol (DSPG), N-(Carbonyl-methoxypolyethylenglycol-2000)-1,2-distearoyl-sn-glycero-3-phosphoethanolamine (mPEG-2000-DSPE, Na-salt), and cholesterol were purchased from Lipoid GmbH, Germany. Other reagents were obtained from Sigma-Aldrich Co. (St Louis, MO, USA). All other chemicals used during the study were reagent-grade and were used with no further purification.

### Cancer Cell Lines

All the cell lines including HT-29 (colorectal cancer cell line), H1975 (non-small cell lung cancer, NSCLC cell line), MSTO-211H (mesothelioma cell line), HCC827 (NSCLC cell line), 786-O (renal cell carcinoma cell line), Caki-1 (renal cell carcinoma cell line) and others, were obtained from American Type Culture Collection [ATCC] (Manassas VA, USA). The cells were cultured by following vendor's recommendation. The medium was supplemented with 10% fetal bovine serum (FBS), 100 U/mL penicillin G sodium, and 100 μg/mL streptomycin sulfate. All cells were incubated at 37° C. in 5% CO₂ atmosphere.

### Liposome Preparation

The general drug liposome preparation methods are listed as below.

Active Drug Loading Method: General steps to prepare the drug loaded liposome through active loading are the following: (1) Lipid hydration and size reduction (2) Dialysis/buffer exchange (3) Drug loading by transmembrane pH gradient or transition metal chelation and finally (4) Adjust pH of final drug loaded liposome suspension. For example, mPEG-2000-DSPE (0-10% mole/mole of total lipid) or DSPG (0-50% mole/mole of total lipid), cholesterol (0-60% mole/mole of total lipid), and DSPC/HSPC (0-80% of total lipid) were dissolved in ethanol and hydrated in an aqueous solution containing one of the following trapping agents: ammonium sulfate, polyphosphate (n=5-18), TEA-SOS, TEA-SBE-β-CD, Tris-SBE-β-CD, or salts of transition metals (e.g., copper gluconate and copper sulfate) at about 50-70° C. The liposome containing mPEG-2000-DSPE for stabilization agent is referred as a “PEGylated liposome”. Liposomes containing DSPG for stabilization is referred as “DSPG liposome”. Subsequently, the organic phase and the aqueous phase was mixed under vigorous stirring for approximately 30 minutes to allow for emulsification. The emulsion was subjected to size reduction (e.g., extrusion) using polycarbonate membranes (50-100 nm) at about 50-70° C. to obtain the desired liposome particle size and particle size distribution (PDI), and then was quickly cooled to yield unloaded liposome. After that, the external trapping agent outside the liposome were washed away by diffusion across a dialysis membrane. To load the drug into liposomes, the unloaded liposome suspension was diluted with desired buffer solution. One or two drugs (e.g., afatinib, nintedanib, crizotinib, abemaciclib, sunitinib, osimertinib) were added into the above unloaded liposome suspension and the drug loading was allowed to proceed for 30-45 min at elevated temperature (50-70° C.) under gentle stirring. This active loading approach typically results in a drug encapsulation efficiency higher than 95%. The architecture of the liposomes loaded with compounds through active loading approach is illustrated in FIG. 5(I).

Sequential Passive and Active Drug Loading Method: Both hydrophilic (water-soluble) drug and lipophilic (poorly water-soluble) drug can be co-loaded into the liposome via a sequential passive and active loading approach. Specifically, the lipophilic compound is passively encapsulated within the lipid bilayer of the liposome first. Subsequently, the hydrophilic drug is loaded into the aqueous core of the liposome via the active loading method. An example of preparation process is described as follows. An organic solution of lipids (e.g., DSPG: 0-70% mole/mole, cholesterol: 0-50% mole/mole, and DSPC: 0-50% mole/mole) together with the lipophilic protein kinase inhibitors (such as dasatinib monohydrate, ceritinib and others) was first prepared. The following organic solvents can be used for such purpose, e.g., methanol, ethanol, or mixture of methanol/chloroform, and others. Then, the organic lipid/drug mixture solution was dried to form a thin film by solvent removal via rotary evaporation in a water bath at 50-60° C. After that, the dry lipid film was hydrated with an aqueous solution containing one of the following trapping agents, such as ammonium sulfate, TEA-SOS, Tris-SBE-β-CD and TEA-SBE-β-CD. The hydration process was allowed to proceed at 50-70° C. for several hours under vigorous stirring to form multilamellar vesicles (MHLVs). The turbid MLV suspensions were then extruded or homogenized at 50-70° C. to obtain the desired liposome particle size and particle size distribution. The external trapping agent and unencapsulated drug outside the liposome were removed by diffusion with dialysis or other separation methods. This liposome suspension was then diluted by a selected buffer solution and heated to about 50-70° C. Water-soluble kinase inhibitors (such as afatinib, crizotinib and Osimertinib, etc.) were subsequently added into the warm liposome suspension at a defined drug to lipid ratio and the drug loading process was allowed to proceed for 30-60 min. at 50-70° C. to obtain co-loaded drug liposome. The architecture of such liposome based on the sequential drug loading method containing both the hydrophilic and the lipophilic compounds is illustrated in FIG. 5 (II).

Passive Drug Loading Method: One or more lipophilic (poorly water-soluble) compound can be loaded into the liposome via the passive drug loading approach. For example, an organic solution of lipids (e.g., DSPG: 0-50% mole/mole, cholesterol: 0-60% mole/mole, and DSPC: 0-50% mole/mole) together with the lipophilic protein kinase inhibitors (such as dasatinib monohydrate, ceritinib and others) was first prepared. The following organic solvents can be used for such purpose, e.g., methanol, ethanol, or mixture of methanol/chloroform, and others. Then, the lipid/drug solution was dried to form a thin film by solvent removal via rotary evaporation in a water bath at 50-60° C. After that, the lipid film was hydrated with a desired buffer solution and the hydration process was allowed to proceed at 50-70° C. for several hours under vigorous stirring to form multilamellar vesicles (MLVs). The turbid MLV suspensions were then extrude or homogenized at 50-70° C. to obtain the desired liposome particle size and particle size distribution. The external trapping agent and unencapsulated drug outside the liposome can be removed by diffusion with dialysis or other methods. The architecture of such liposome co-loaded with the lipophilic compounds is illustrated in FIG. 5 (III).

### Characterization of Liposomes

Particle Size and Zeta (ζ)-Potential. Hydrodynamic particle size, polydispersity index (PDI) and the zeta-potential of the liposome drug product were measured using a Nano-S90 ZetaSizer (Malvern Instruments, UK). Each sample was adequately diluted with distilled water prior to measurement.

Morphological Characterization. Cryo-Transmission Electron Microscopy (Cryo-TEM) was employed to examine the size and morphology of the co-loaded liposome using a Cryogenic TEM-Titan Krios 80/300 Kev transmission electron microscope (ThermoFisher Scientific).

Drug Loading and Encapsulation. Drug content (Assay) of the liposome product was determined by dissolving a known quantity of loaded liposome in Triton-X100 aqueous solution and the drug content was quantified by HPLC-UV analysis. The free drug content was determined by first separating the free drug from the liposome through size exclusion chromatography (SEC), and then the unloaded drug content was quantified by HPLC-UV analysis. The encapsulation efficiency (EE %) was calculated as the free drug content subtracted from the total drug content divided by the total drug content.

In-vitro drug release study. In-vitro release of drug loaded liposomes can be evaluated through a dialysis-based approach. For example, a defined volume of liposome product (˜1-2 mL) was first added into a dialysis bag (molecular weight cutoff 10 kDa), which was pre-hydrated overnight in phosphate-buffered saline (PBS) buffer at pH 7.4. The dialysis bag was then placed into a glass reservoir containing 150 mL PBS (pH 7.4). The dissolution study was allowed to proceed at 37° C. under gentle stirring. Aliquots (˜1 mL) of the release media were sampled at predetermined time intervals and the reservoir was replenished with equal volumes of fresh media. The drug content of the specific compound was determined by HPLC-UV method. Cumulative drug release profile was then generated based on the released drug content at each time point.

### In-Vitro Cytotoxicity Study for Drug Synergy Determination

For combined drug regimens, the two or more compounds involved in the combination can exhibit synergistic, additive, or antagonistic interactions depending on the molar drug ratios. To study those drug-drug interactions in a quantitative approach, a combination index (CI)-based method was used in this study following previously reported procedure (Chou, T. C., J. Theor. Biol. (1976) 39:253-276).

The general protocol on cell culture and liposome drug efficacy evaluation were briefly stated as follows. Adherent cancer cell lines were collected in their logarithmic growth phase using standard cell culture techniques. Cell concentrations were determined by hemocytometer and then diluted by their respective media to the targeted cell concentrations. Cells were then seeded onto a 96-well plate. The map on the plate was designed to include treatment groups, cell-only control (no drug treatment) and media-only control (no cell and no drug treatment). Cell seeding concentrations were optimized such that 48 hours after cell plating a MTT assay performed on the untreated control cells would generate an absorbance value of around 1.0 at 590 nm.

The cell seeded plate was incubated for 24 hours at 37° C. and 5% CO₂ in a standard cell culture incubator before drug treatment. The following day, drug dilutions on either solo drug or drug combinations at defined molar drug ratios were prepared using respective cell culture media. The cell culture media in the 96-well plate was then replaced by fresh media containing the drug or drug combinations. After another 24 hours of incubation, cell viability was assessed by the MTT assay following the manufacture's protocols. Relative percent survival was determined by subtracting absorbance values obtained by media-only wells from drug treated wells and then normalizing to the no-drug control wells (cell only control). The fraction of cells affected (fa), or cell growth inhibition (%) at each drug concentration was subsequently calculated for each well. The effect of drug combinations was then calculated and processed by a software CompuSyn for drug synergy analysis. The program employs the median-effect analysis algorithm, which produces the Combination Index value as a quantitative indicator of the degree of synergy. Based on this analysis method, a CI<0.9 indicates synergy, the range 0.9≤CI≤1.1 reflects additive effect and a CI >1.1 indicates antagonism. CI plots are typically illustrated with CI representing the y-axis versus the proportion of cells affected, or fraction affected (Fa), on the x-axis. The synergistic ratio of drug combinations was identified and then used for future studies.

### In-Vitro Tumor Cell Growth Inhibition by Drug-Loaded Liposomes

To study in-vitro inhibition on cell growth by liposome drug product, cancer cells were treated with both combo drug-loaded liposomes (contain synergistic drug to drug molar ratios) and the corresponding single drug loaded liposomes. A brief experimental procedure is stated as follows. Cancer cells were seeded onto the 96-well plate with appropriate seeding density. The cell seeded plate was incubated for 24 hours at 37° C. and 5% CO₂ in a standard cell culture incubator before drug treatment. The following day, serial dilutions on liposome drug product were prepared using respective cell culture media. The cell culture media in the 96-well plate was then replaced by fresh media containing liposome encapsulated drugs. After a total of 48 hours of incubation, cell viability was assessed by the MTT assay following the manufacture's protocols. Relative percent survival was determined by subtracting absorbance values obtained by media-only wells from drug treated wells and then normalizing to the no-drug control wells (cell only control). The percentage of cell growth inhibition is calculated by subtracting the % of cell viability from 100%.

### In-Vivo Efficacy Study

Development of HT-29 and H1975 Xenograft Models. Six-week-old female BALB/c nude mice were subcutaneously injected in the right flank region with ˜1×10⁷ HT-29 colorectal cancer or H1975 NSCLC cells dispersed in 100 μL of PBS buffer. When the tumor sizes reached ˜150-200 mm³, the mice were divided randomly into 6 groups with six mice in each group (n=6). The mice were kept at 20±2° C. and 50-60% relative humidity throughout the study period. All animal-handling procedures were in accordance with the protocols approved by the Institutional Animal Ethical Committee.

Drug Administration and Tumor Size Measurement. When the tumor size reached the desired size as stated above, the following samples were then administrated through tail vein intravenous (IV) injection into the tumor bearing mice: (1) saline control (2) free AFA solution (3) free AFA/NIN combo solution (4) liposome AFA (AFA-L) (5) liposome NIN (NIN-L) and (6) combo AFA/NIN-L. For each drug formulation, the following dose was employed: 7.0 mg/kg for AFA as free base and 38.9 mg/kg for NIN as free base. The efficacy study was performed for about 20 days with the drug formulation being administrated every two days (Q2D). Tumor sizes were measured twice weekly in two dimensions using a caliper, and the volume was expressed in mm³ using the formula: V=0.5*(a)*(b²), where a and b are the long and short diameters of the tumor, respectively.

Statistical Data Analysis. Statistic differences between the co-loaded drug liposome treated group and other treatment groups during MTT assay and in-vivo antitumor study were determined by one-way ANOVA combined with Dunnett's test at the significance level of p<0.05. All observations were expressed as mean±SD (n=6).

### EXAMPLES

The following examples are provided to illustrate, but not to limit, the invention disclosed.

### Example 1

### Preparation and Physical Characterization of Afatinib Liposome

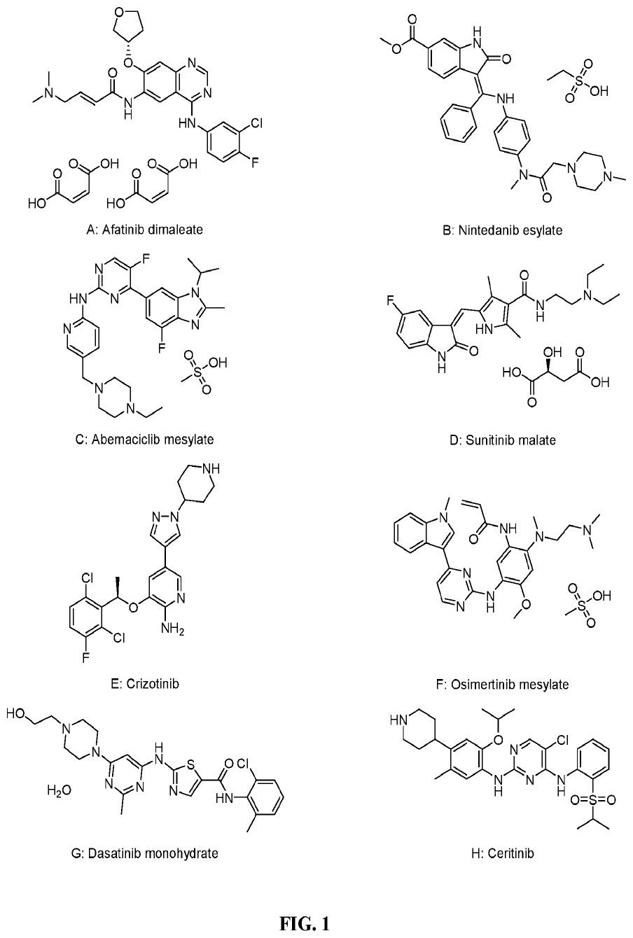

Afatinib liposome (AFA-L) was prepared by the active drug loading approach as described in the Experimental Methods. The structure of afatinib dimeleate is shown in FIG. 1A. Systematic experiments were conducted to identify and optimize major factors in formulation and production process conditions (such as lipid selection, composition of liposome, drug to lipid ratio, trapping agent, and process conditions, etc.) that affect the physicochemical properties of the liposome (e.g., liposome particle size, particle size distribution, encapsulation efficiency, liposome stability, drug release profile, and others).

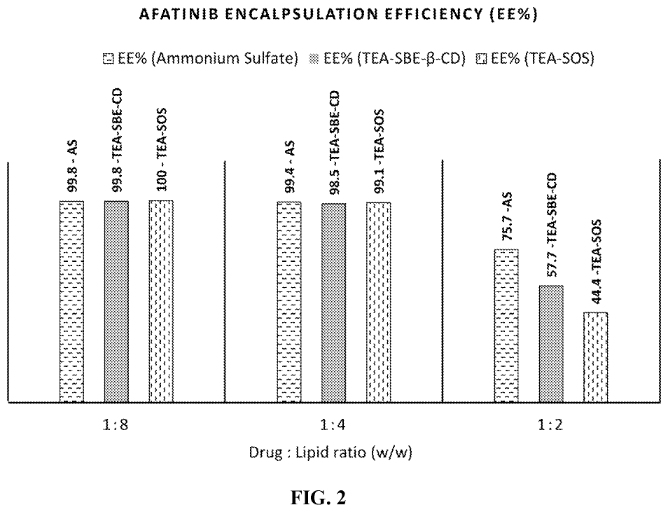

Specifically, AFA-L was prepared based on a lipid composition of HSPC/mPEG-2000-DSPE/Cholesterol using the following trapping agents to actively load the compound into the liposome: ammonium sulfate, TEA-SOS or TEA-SBE-β-CD. Overall, the PEGylated AFA-L exhibits the following physical properties: average particle size around 90 nm, PDI<0.100, encapsulation efficiency (EE %)>95.0%, and ζ-Potential (surface charge)<−40 mV. The EE % of the afatinib liposome using various trapping agents are shown in FIG. 2. The results reflect that the drug to lipid ratio has a significant effect on the EE % of the payload. Namely, when the ratio was at 1:8 or 1:4 (w/w), close to 99% of EE % was obtained for AFA-L among all three trapping agents. However, when the drug to lipid weight ratio was at 1:2, a drastic drop in afatinib EE % was observed for all liposomes independent on trapping agent used. Lipid to drug ratio can affect to liposome capacity as well as internal trapping agent availability, and the result indicates that a sufficient amount of lipid is critical to ensure a high drug encapsulation efficiency can be obtained.

In addition, the negative charged DSPG lipid was also used to prepare liposomes for the encapsulation of afatinib. The liposome manufacture process was mentioned in the Experimental Methods. Specifically, the afatinib-loaded DSPG liposomes was formulated using the following trapping agent to actively encapsulate the payload into the liposomes, i.e., ammonium sulfate, TEA-SOS, and TEA-SBE-β-CD. The typical afatinib-loaded DSPG liposomes has the following physical parameters: average particle size around 110 nm, PDI<0.100, encapsulation efficiency (%)>95.0%, and ζ-Potential <−30 mV.

### Example 2

### Preparation and Characterization of Nintedanib Liposome

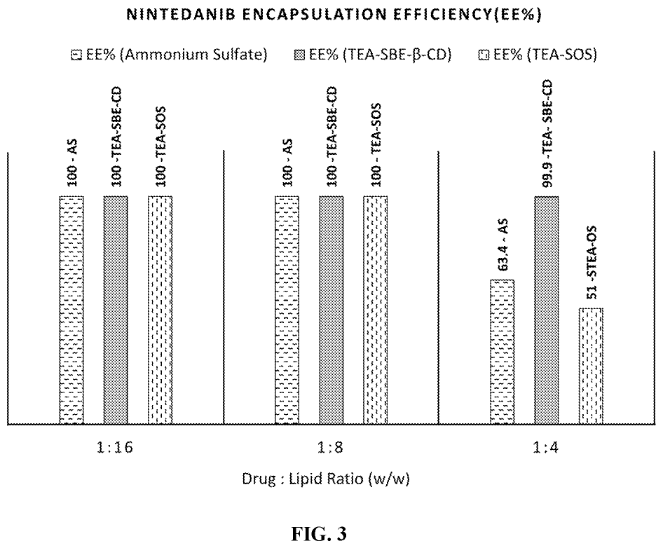

Systematic studies were conducted to identify and optimize major factors in formulation and production process that can affect the physicochemical properties of the drug-loaded liposome. Nintedanib liposomes (NIN-L) was prepared by the active loading method as described in the Experimental Methods. The structures of nintedanib esylate is shown in FIG. 1B. FIG. 3 shows the effect of both trapping agent and drug to lipid weight ratio on the drug encapsulation efficiency (EE %). When using TEA-SBE-β-CD as the trapping agent, a high EE % (close to 100%) was obtained for all drug to lipid ratio tested. In contrast, when TEA-SOS and ammonium sulfate were used as the trapping agent, high EE % was only observed at the drug to lipid ratio (w/w) of 1:16 and 1:8. When the drug to lipid ratio was increased to 1:4, a significant drop in EE % was obtained for liposomes with TEA-SOS and ammonium sulfate. The above results reflect that among all the trapping agents evaluated, TEA-SBE-β-CD shows superior properties for the encapsulation of nintedanib.

In addition, the negative charged DSPG was also used to prepare liposomes for the encapsulation of nintedanib. The liposome manufacture process was mentioned in the experimental section. Specifically, the nintedanib-loaded DSPG liposomes was formulated using the following trapping agent for active encapsulation of the payload, i.e., ammonium sulfate, TEA-SOS, TEA-SBE-β-CD. The typical nintedanib-loaded DSPG liposomes has the following physical parameters: average particle size around 120 nm, PDI<0.100, encapsulation efficiency (%)>95.0%, and ζ-Potential <−30 mV.

### Example 3

### Preparation of TEA-SOS and TEA-SBE-β-CD Trapping Agents

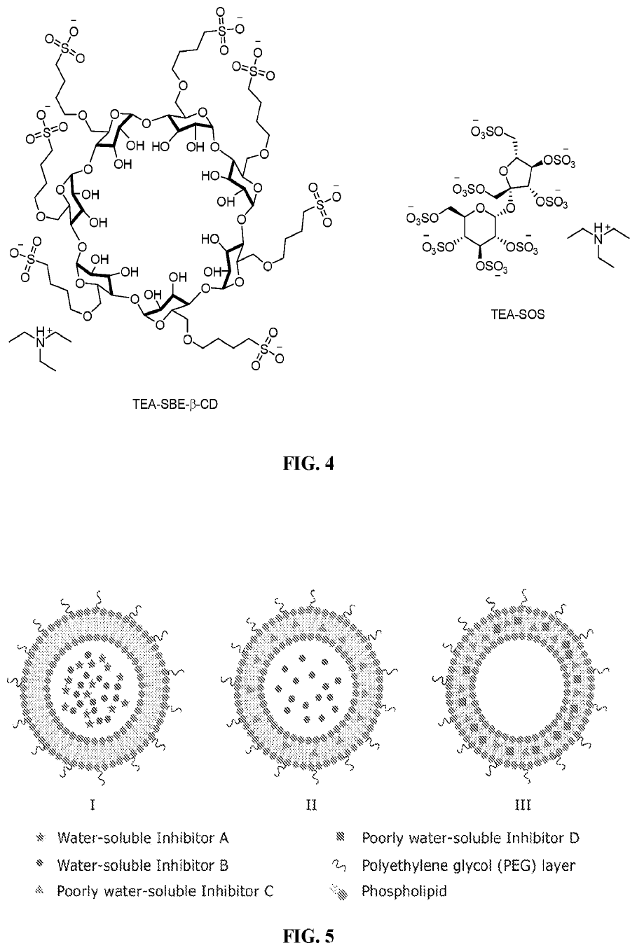

It is known that the trapping agent involved in active loading plays a critical role on payload encapsulation, retention as well as its dissolution profiles. In addition to the commonly used ammonium sulfate, polyanion based trapping agents were explored here for the encapsulation of kinase inhibitors. The structure of two polyanions involved in this work, i.e., TEA-SOS and TEA-SBE-β-CD, were shown in FIG. 4.

Preparation of TEA-SOS and TEA-SBE-β-CD: An ion exchange column was first packed with sulfonated polystyrene-divinylbenzene copolymer-based cation exchange resin beads. Then, the resin was equilibrated with ˜1N HCl, and subsequently washed with deionized water until the pH of the eluate was close to neutral. After that, solution of the sodium salt of sucrose octasulfate (SOS) or SBE-β-cyclodextrin was added to the column and eluted with deionized water. The eluate was then titrated with triethylamine to a pH of 4.0-6.0. In some cases, tris(hydroxymethyl) aminomethane (Tris) was used as the base for titration to generate Tris salt of the polyanion, e.g., Tris-SEB-β-cyclodextrin or Tris-SOS.

### Example 4

### Preparation of Afatinib and Nintedanib Co-Loaded Liposome

The co-loaded PEGylated liposome (PEGylated AFA/NIN-L) was prepared by the active loading method via transmembrane pH gradient as described in the Experimental Methods. The lipid composition is based on mPEG-2000-DSPE/cholesterol/DSPC or mPEG-2000-DSPE/cholesterol/HSPC. Various types of trapping agents were used for the preparation of such dual drug-loaded liposome, i.e., ammonium sulfate, TEA-SBE-β-CD, Tris-SBE-β-CD, TEA-SOS, copper gluconate/TEOA. In the drug loading step, AFA and NIN at a defined molar ratio (e.g., AFA to NIN at 1:10, 1:5 or 1:1) were introduced into the liposome suspension. The scheme of the PEGylated AFA/NIN-L is shown in FIG. 5 (I). Systematic experimentation was conducted to identify and optimize major factors in formulation and production process conditions (such as lipid selection, composition of liposome, drug to lipid ratio, drug to drug molar ratios, trapping agent, and process conditions, etc.) that affect the physicochemical properties of the liposome (e.g., liposome particle size, particle size distribution, encapsulation efficiency, liposome stability, drug release profile, and others).

In addition to the PEGylated liposomes, DSPG liposome was also developed for the encapsulation of protein kinase inhibitors. In this case, the lipid composition is based on DSPG, cholesterol and DSPC or HSPC. Different types of trapping agents, such as ammonium sulfate, can be used for encapsulation of the actively loaded drug. The detailed liposome preparation method is described in the Experimental Methods section (Active Drug Loading Method). As illustrated in FIG. 5 (I), both inhibitors are located inside the aqueous core compartment of the liposome.

### Example 5

### Physicochemical Characterization of AFA/NIN Liposomes

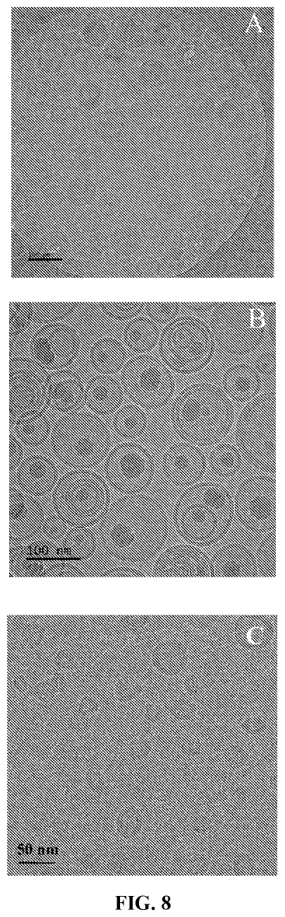

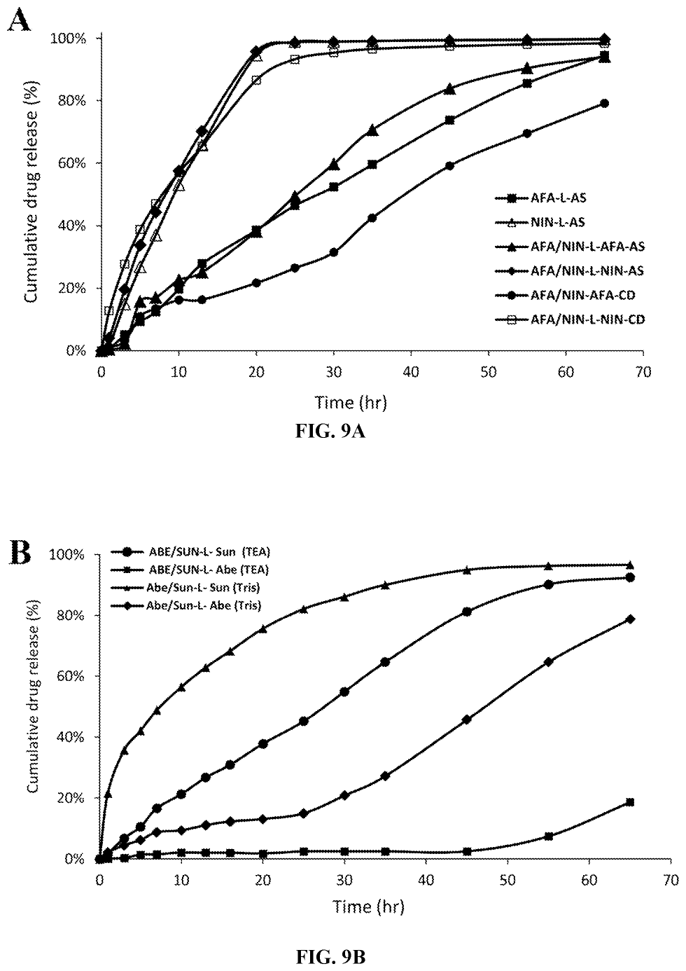

Physicochemical characterization on the AFA/NIN co-loaded liposomes was performed. The drug encapsulation efficiency of the liposome product using the above-mentioned trapping agents (Example 4) are summarized in Table 1 below. As reflected from the results, all the trapping agents except copper gluconate/TEOA gave very high EE % (˜99%) for both AFA and NIN at all three drug molar ratios tested. A significantly lower EE % was obtained when copper gluconate/TEOA was used as the trapping agent. Thereby, for the co-encapsulation of AFA and NIN, the results indicate that the pH gradient-based loading method is superior to transition metal-based approach in terms of drug encapsulation. Particle size and size distribution of AFA/NIN co-loaded PEGylated liposome using ammonium sulfate and TEA-SBE-β-CD as the trapping agent were shown in FIG. 6 and FIG. 7, respectively. All of the combo drug liposomes exhibited a mean particle size around 100 nm with a polydispersity (PDI)<0.100 which indicates a narrow size distribution.

TABLE 1
Effect of both drug molar ratio and trapping agent on physicochemical
properties of AFA/NIN co-loaded PEGylated liposome

| Sample Name | | EE % | | EE % | | EE % | | EE % | | EE % Cu | |
|---|---|---|---|---|---|---|---|---|---|---|---|
| AFA:NIN | Mean Particle | (NH₄)₂SO₄ | | TEA- SBE-β-CD | | TEA-SOS | | Tris-SBE-β-CD | | Gluconate/TEOA | |
| (Molar Ratio) | Size (nm)/PDI | AFA | NIN | AFA | NIN | AFA | NIN | AFA | NIN | AFA | NIN |

| AFA/NIN-L 1:10 | 110/<0.1 | 99.4 | 100 | 100 | 99.4 | 99.7 | 100 | — | | — |
|---|---|---|---|---|---|---|---|---|---|---|
| AFA/NIN-L 1:5 | 110/<0.1 | 99.5 | 99.9 | 100 | 99.3 | 99.8 | 100 | 99.5 | 99.8 | — |

| | | | | | | | | | | |
|---|---|---|---|---|---|---|---|---|---|---|
| AFA/NIN-L 1:1 | 110/<0.1 | 99.8 | 100 | 100 | 98.9 | 99.9 | 99.8 | — | 70.7 | 88.4 |

The effect of lipid to drug molar ratio on both drug encapsulation efficiency (EE %) and particle size are shown in Table 2 below. It was found that as the lipid to drug molar ratio decreased, the EE % for both AFA and NIN were slightly reduced. But an overall higher than 9500 of EE % was obtained for all conditions studied (Table 2). Also, it was observed that the liposome particle size was decreased as the lipid to drug molar ratio in the formulation was reduced.

TABLE 2
Effect of lipid to drug weight ratio on Encapsulation Efficiency (EE %) and particle
size of the AFA and NIN co-loaded PEGylated liposome with TEA-SBE-β-CD trapping agent

| Sample Name | AFA:NIN | Total Lipid to Total | Mean Particle Size | EE % | |
|---|---|---|---|---|---|
| (AFA/NIN-L) | Molar Ratio | Drug Ratio (w/w) | (nm)/PDI | AFA | NIN |
| 1130-1 | 1:1 | 7.5:1 | 100/<0.1 | 100 | 98.9 |
| 1130-2 | 1:5 | 7.3:1 | 100/<0.1 | 100 | 99.3 |
| 1130-3 | 1:1 | 3.8:1 | 91/<0.1 | 99.7 | 99.2 |
| 1130-4 | 1:5 | 3.6:1 | 92/<0.1 | 99.5 | 99.6 |
| 1130-5 | 1:1 | 1.9:1 | 89/<0.1 | 95.8 | 96.4 |
| 1130-6 | 1:5 | 1.8:1 | 91/<0.1 | 98.0 | 98.7 |

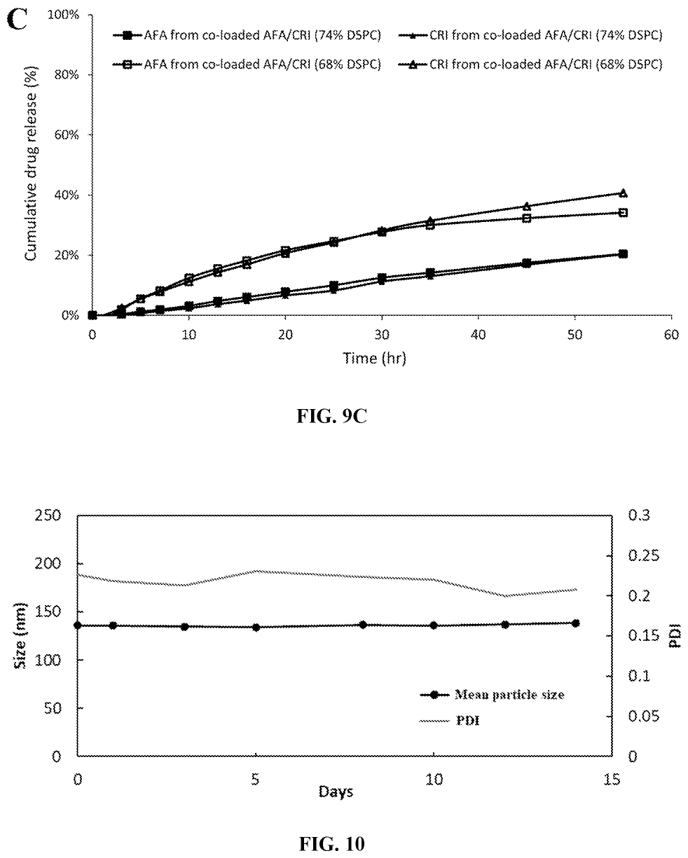

Cryo-TEM was performed to visualize the morphology of the AFA/NIN co-loaded liposome using TEA-SOS as the trapping agent at 1:5 molar ratio of AFA to NIN. As shown in FIG. 8A, the result revealed that the liposome was spherical in shape and a dark stripe-like structure was observed inside the aqueous core of the liposome. It is believed that the dark precipitates were the complex formed by the drug and the trapping agent. On average, the particle size (80-100 nm) revealed from TEM agrees with the result found by dynamic light scattering. Those results confirm that the drugs were well encapsulated inside of the aqueous core compartment of the liposome in a precipitated state.

Comparing to Pegylated AFA/NIN-L liposome, the DSPG-AFA/NIN-L liposomes have around 137 nm mean particle size and 0.099 PDI with trapping agent ammonium sulfate at AFA:NIN drug molar ratio 1:1. The encapsulated efficiency (%) of afatinib and nintedanib in the DSPG-AFA/NIN-L are 68% and 85%, respectively. The results reflected that an acceptable level of EE % can be obtained for all the payload, and a particle size of less than 150 nm with narrow polydispersity for both co-loaded liposomes. However, the EE % with DSPG-AFA/NIN-L is not as good as in the PEGylated-AFA/NIN-L liposome.

### Example 6

### AFA/NIN-L In-Vitro Release and Stability Studies

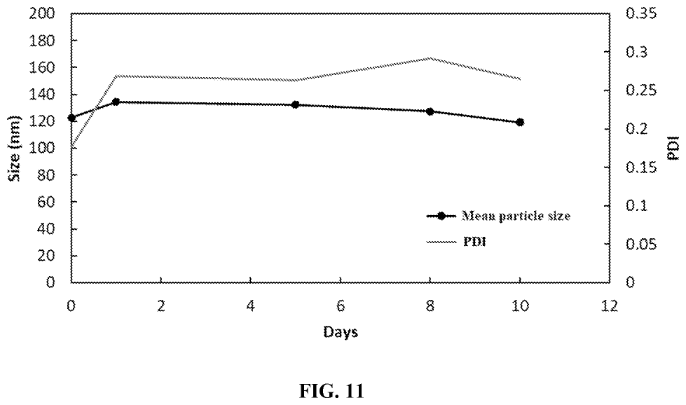

Drug release studies from AFA/NIN co-loaded PEGylated liposomes (AFA/NIN-L at 1:5 molar drug ratio using ammonium sulfate or TEA-SBE-β-CD as trapping agents), along with studies from PEGylated AFA-L and NIN-L, were performed at pH 7.4 PBS buffer solution under accelerated conditions at 45° C. by dialysis. Overall, as shown in FIG. 9A, a sustained release profile was obtained for both AFA and NIN from the liposomes and the release rate of AFA was faster than that of NIN. The release rate of the payload from the co-loaded liposome was comparable to that of the corresponding API from the single drug-loaded liposome using the same type of trapping agent, which indicates that the co-encapsulation of the AFA and NIN into one liposome did not change their dissolution profiles. Also, the result in FIG. 9A reflects that the liposome using TEA-SBE-β-CD as the trapping agent exhibited superior retention on AFA as compared to that of liposomes using ammonium sulfate. This indicates that AFA may have stronger interaction with TEA-SBE-β-CD which leads to slower drug release profile.

Physical stability of the PEGylated AFA/NIN co-loaded liposome using ammonium sulfate as the trapping agent was assessed by recording the changes in particle size, particle size distribution (PDI), and encapsulation efficiency (EE %) over a period of 45 days upon storage at 4° C. (long-term storage condition) and 25° C. (accelerated condition). An aliquot of the sample was taken at initial, 1, 4, 8, and 16 weeks of storage and particle size characterization and EE % were determined by dynamic light scattering and HPLC analysis, respectively. The results shown in Table 3 indicated that the AFA/NIN co-loaded liposome was physically stable for 45 days at both 4° C. and 25° C. storage conditions.

TABLE 3
Physical Stability of PEGylated AFA/NIN-L (Ammonium Sulfate was sed as the trapping
agent) at both 4° C. (long-term storage) and 25° C. (accelerated) storage conditions

| | Storage at 4° C. | | | Storage at 25° C. | | |
|---|---|---|---|---|---|---|
| | Mean Particle Size | Drug Content | | Mean Particle | Drug Content | |
| Week | (nm)/PDI | (mg/mL) | EE % | Size (nm)/PDI | (mg/mL) | EE % |
| 0 | 87/<0.1 | 1.05 | 99.3 | 87/<0.1 | 1.05 | 99.3 |
| 1 | 87/<0.1 | 1.01 | 99.6 | 87/<0.1 | 1.02 | 99.6 |
| 4 | 87/<0.1 | 1.0 | 99.7 | 87/<0.1 | 1.0 | 99.5 |
| 8 | 88/<0.1 | 0.99 | 99.6. | 86/<0.1 | 0.99 | 99.5 |
| 16 | 87/<0.1 | 0.98 | 99.7 | 87/<0.1 | 1.0 | 99.7 |

### Example 7

### Preparation and Characterization of Abemaciclib and Sunitinib Co-Loaded Liposome

Liposomes co-loaded with abemaciclib (ABE, CDK family inhibitor) and sunitinib (SUN, PDGFR α/β inhibitor) were prepared based on the procedure mentioned in the Experimental Methods (Active Drug Loading Method). The structures of abemaciclib mesylate and sunitinib malate are described in FIG. 1 (C, D). Both PEGylated liposome (mPEG-2000-DSPE/cholesterol/DSPC) and DSPG liposome (DSPG/cholesterol/DSPC) were used for active drug loading (see Experimental Methods). Different types of trapping agent were used for the dual drug encapsulation, i.e., TEA-SOS, TEA-SBE-β-CD and Tri-SBE-β-CD. In the drug loading step, both ABE and SUN at a defined molar ratio (e.g., 1 to 5) were introduced into the liposome suspension for active drug loading. The scheme of the PEGylated liposome co-loaded with those two inhibitors is shown in FIG. 5 (I). As illustrated by the scheme, both of the drugs are located inside the aqueous core compartment of the liposome. The physicochemical characterization of both the single drug loaded liposomes and the ABE/SUN co-loaded liposomes using different types of trapping agents were shown in Table 4. High drug encapsulation efficiency (>99%) was obtained for all drug products. A particle size around 100 nm with narrow particle size distribution (PDI<0.1) was observed for all types of the liposomes (Table 4).

TABLE 4
Physicochemical characterizations on PEGylated liposomes of ABE-L, SUN-L, and ABE/SUN-L

| | Molar Ratio | Trapping | Mean Particle | Zeta-potential | EE % | |
|---|---|---|---|---|---|---|
| Sample Name | (ABE:SUN) | Agent | Size (nm)/PDI | (mV) | ABE | SUN |
| ABE-L-SOS | — | TEA-SOS | 102/0.072 | −20.6 | 99.8 | — |
| SUN-L-SOS | — | TEA-SOS | 105/0.083 | −34.1 | — | 99.8 |
| ABE/SUN-L-SOS | 1:5 | TEA-SOS | 102/0.033 | −36.5 | 99.5 | 99.7 |
| ABE/SUN-L-TEA-CD | 1:5 | TEA-SEB-β-CD | 94/0.047 | −35.3 | 99.5 | 99.6 |
| ABE/SUN-L-Tris-CD | 1:5 | Tris-SEB-β-CD | 98/0.050 | −35.8 | 99.8 | 99.2 |

The dissolution profile of ABE/SUN co-loaded PEGylated liposomes was also studied. As shown in FIG. 9B, the release rate of ABE was slower than that of SUN regardless of the trapping agent employed. Also, for the same drug, liposomes using TEA-SBE-β-CD as the trapping agent exhibited a slower drug release rate as compared to that from liposomes using Tris-SBE-β-CD. The results indicate that the counter-ion to the polyanion in the trapping agent plays an important role in controlling the drug release rate, and the TEA is superior to Tris in terms of retaining the drug within the liposome.

In addition to the PEGylated liposomes, DSPG liposome was also developed for the encapsulation of protein kinase inhibitors. In this case, the lipid composition is based on DSPG, Cholesterol and DSPC or HSPC. Different types of trapping agents, such as ammonium sulfate, can be used for encapsulation of the actively loaded drug. The detailed liposome preparation method is described in the Experimental Methods section (Active Drug Loading Method). As illustrated in FIG. 5 (I), both inhibitors are located inside the aqueous core compartment of the liposome. Comparing to Pegylated ABE/SUN-L liposome, the DSPG-ABE/SUN-L liposomes have around 149 nm average particle size and 0.166 PDI with trapping agent ammonium sulfate at ABE:SUN drug molar ratio 1:1. The encapsulated efficiency (%) of abemaciclib and sunitinib in the DSPG-ABE/SUN-L are 91% and 85%, respectively. The results reflected that an acceptable level of EE % can be obtained for all the payload, and a average particle size around 150 nm with narrow polydispersity was obtained for both co-loaded liposomes. However, the EE % with DSPG-ABE/SUN-L is not as good as in the Pegylated-AFA/NIN-L liposome.

### Example 8

### Preparation and Characterization of Afatinib and Crizotinib Co-Loaded Liposome

Liposomes co-loaded with afatinib (AFA, EGFR inhibitor) and crizotinib (CRI, ALK inhibitor) were prepared based on the procedure mentioned in the Experimental Methods (Active Drug Loading Method). The structure of afatinib dimaleate and crizatinib are described in FIG. 1 (A and E). Both PEGylated liposome (mPEG-2000-DSPE/cholesterol/DSPC) and DSPG liposome (DSPG/cholesterol/DSPC) were used for the active loading of the dual drugs (see Experimental Methods). Different types of trapping agent were used for the dual drug encapsulation, i.e., TEA-SOS, TEA-SBE-β-CD and Tri-SBE-β-CD. In the drug loading step, both AFA and CRI at a defined molar ratio (e.g., 1 to 1) were introduced into the liposome suspension for active drug loading. The scheme of the PEGylated liposome co-loaded with those two inhibitors is shown in FIG. 1 (I). As illustrated by the scheme, both inhibitors are located inside the aqueous core compartment of the liposome. The results of the physicochemical characterization on both the single drug loaded liposomes and the AFA/CRI co-loaded PEGylated liposomes were summarized in Table 5. The data reflects that both inhibitors were efficiently encapsulated in the liposome for all trapping agents evaluated (>99% EE %). The average particle size of AFA/CRI co-loaded liposomes were around 100 nm, and a narrow size distribution was obtained (PDI<0.1).

TABLE 5
Physicochemical Characterizations on PEGylated liposomes of AFA-L, CRI-L, and AFA/CRI-L

| | Molar Ratio | | Mean Particle | Zeta-potential | EE % | |
|---|---|---|---|---|---|---|
| Sample Name | AFA:CRI | Trapping Agent | Size (nm)/PDI | (mV) | AFA | CRI |
| AFA-L-SOS | — | TEA-SOS | 110/0.103 | −25.7 | 96.8 | — |
| CRI-L-SOS | — | TEA-SOS | 112/0.035 | −36.5 | — | 99.8 |
| AFA/CRI-L-SOS | 1:1 | TEA-SOS | 105/0.043 | −45.4 | 99.5 | 99.6 |
| AFA/CRI-L-Tris-CD | 1:1 | Tris-SEB-β-CD | 98/0.049 | −42.8 | 99.4 | 99.7 |
| AFA/CRI-L-TEA-CD | 1:1 | TEA-SEB-β-CD | 94/0.033 | −44.6 | 99.8 | 99.6 |

The dissolution profiles of AFA/CRI co-loaded PEGylated liposomes were also studied. As shown in FIG. 9C, the data reflects that at the fixed lipid composition, AFA and CRI exhibited a similar release rate. For the same drug, the lipid composition that includes a high DSPC content 74% (w) significantly slowed down the drug release rate as compared to that from the lipid composition where the DSPC content is 68% (w). The results indicate that the content of the bilayer-forming lipid DSPC plays an important role in drug retention within the liposome.

### Example 9

### Preparation and Characterization of Osimertinib and Afatinib Co-Loaded Pegylated Liposome

The liposomes co-loaded with two EGFR targeted kinase inhibitors osimertinib (OSI) and afatinib (AFA) were prepared based on the procedure mentioned in the Experimental Methods (Active Drug Loading Method). The chemical structure of osimertinib mesylate and afatinib dimaleate are shown in FIG. 1F and FIG. 1A, respectively. The PEGylated liposome formulations (mPEG-2000-DSPE/cholesterol/DSPC) were used for the liposome preparation. The scheme of the PEGylated liposome co-loaded with those two inhibitors is shown in FIG. 1 (I). Four different types of trapping agents were used for each of the co-loaded liposomes including ammonium sulfate, Tris-SBE-β-CD, TEA-SBE-β-CD, and TEA-SOS. In the drug loading step, both OSI and AFA at a defined molar ratio (e.g., 1 to 1) were introduced into the liposome suspension for active drug loading. As illustrated by the scheme, both inhibitors are located inside the aqueous core compartment of the liposome. The physicochemical properties of both the co-loaded liposome and its corresponding single drug-loaded liposome are shown in Table 6. As reflected from the results, the OSI/AFA co-loaded liposome exhibited a very high encapsulation efficiency for both of the payloads for all the trapping agent employed. The particle size of the drug loaded liposomes are in a range of around 100 nm with a low polydispersity index (PDI<0.1) for all the liposomes.

TABLE 6
Physicochemical Characterizations on PEGylated liposomes of OSI-L, AFA-L, and OSI-L/AFA-L

| | Molar Ratio | | Mean Particle | Zeta-potential | EE % | |
|---|---|---|---|---|---|---|
| Sample Name | (OSI:AFA) | Trapping Agent | Size (nm)/PDI | (mV) | OSI | AFA |
| OSI-L | — | TEA-SOS | 106/0.068 | −24.6 | 100 | — |
| AFA-L | — | TEA-SOS | 110/0.103 | −25.7 | — | 96.8 |
| OSI/AFA-L-SOS | 1:1 | TEA-SOS | 107/0.045 | −34.9 | 99.9 | 99.7 |
| OSI/AFA-L-Tris-CD | 1:1 | Tris-SEB-β-CD | 97/0.075 | −33.6 | 94.6 | 94.9 |
| OSI/AFA-L-TEA-CD | 1:1 | TEA-SEB-β-CD | 92/0.069 | −35.9 | 99.9 | 99.8 |
| OSI/AFA-L-AS | 1:1 | (NH₄)₂SO₄ | 113/0.060 | −15.8 | 99.8 | 99.7 |

Cryo-TEM was performed to observe the morphology of the OSI/AFA co-loaded PEGylated liposome with a OSI to AFA molar ratio of 1:1. TEA-SOS was used as the trapping agent for this liposome drug product. As revealed from the TEM image (FIG. 8B), round and distinct drug precipitates were formed in the aqueous core of the liposome. Results from TEM characterization demonstrated that the inhibitors were successfully encapsulated within the liposome.

### Example 10

### Preparation and Characterization of Osimertinib and Crizotinib Co-Loaded Pegylated Liposome

The liposomes co-loaded with EGFR inhibitor osimertinib (OSI) and ATK inhibitor crizotinib (CRI) were prepared based on the procedure mentioned in the Experimental Methods (Active Drug Loading Method). The PEGylated liposome formulation with a lipid composition of mPEG-2000-DSPE/cholesterol/DSPC was used for the liposome preparation. Four different types of trapping agents were used for active drug loading including ammonium sulfate, Tris-SBE-β-CD, TEA-SBE-β-CD, and TEA-SOS. In the drug loading step, both OSI and CRI at a defined molar ratio (e.g., 1 to 1) were introduced into the liposome suspension for active drug loading. The scheme of the PEGylated liposome co-loaded with those two inhibitors is shown in FIG. 5 (I). As illustrated by the scheme, both inhibitors are located inside the aqueous core compartment of the liposome. The physicochemical properties of these co-loaded drug liposomes are shown in Table 7. As reflected from the results, the OSI/CRI co-loaded liposome exhibited a very high encapsulation efficiency for both of the payload for all the trapping agent employed except Tris-SBE-β-CD. The particle size of the drug loaded liposomes are in a range of around 100 nm with a low polydispersity index (PDI<0.1) for all the co-loaded liposomes.

TABLE 7
Physicochemical Characterizations on PEGylated liposomes of OSI-L, CRI-L, and OSI-L/CRI-L

| | Molar Ratio | | Mean Particle | Zeta-potential | EE % | |
|---|---|---|---|---|---|---|
| Sample Name | (OSI:CRI) | Trapping Agent | Size (nm)/PDI | (mV) | OSI | CRI |
| OSI-L | — | TEA-SOS | 106/0.068 | −24.6 | 100% | — |
| CRI-L | — | TEA-SOS | 112/0.035 | −36.5 | — | 99.8 |
| OSI/CRI-L-SOS | 1:1 | TEA-SOS | 110/0.089 | −40.8 | 99.5 | 99.4 |
| OSI/CRI-L-Tris-CD | 1:1 | Tris-SEB-β-CD | 99/0.116 | −36.7 | 87.4 | 78.9 |
| OSI/CRI-L-TEA-CD | 1:1 | TEA-SEB-β-CD | 98/0.098 | −35.9 | 99.9 | 99.1 |
| OSI/CRI-L-AS | 1:1 | (NH₄)₂SO₄ | 111/0.082 | −14.7 | 99.1 | 85.7 |

### Example 11

### Preparation and Characterization of Dasatinib and Afatinib Co-Loaded DSPG Liposomes

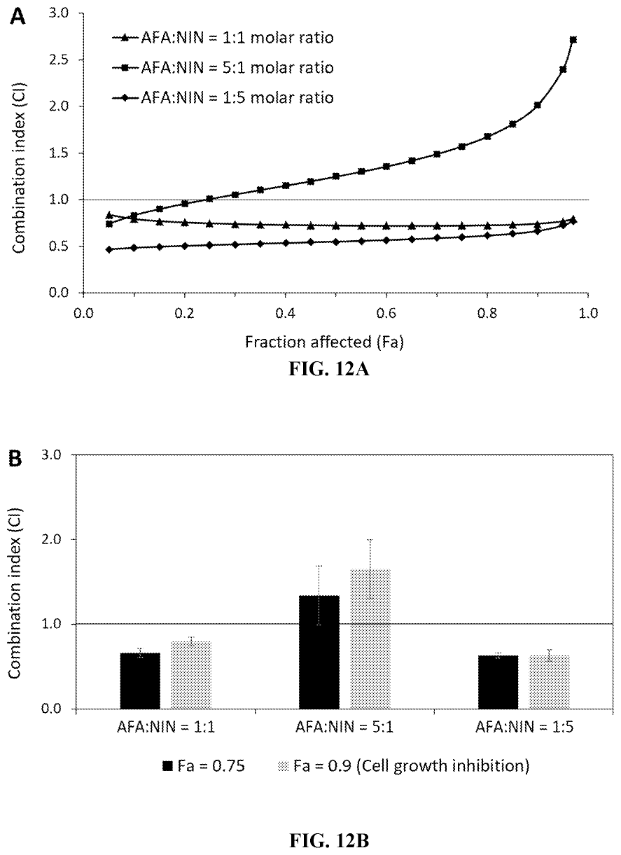

As illustrated from Scenario II of dual-drug encapsulation shown in FIG. 5 (II), a poorly water-soluble lipophilic protein kinase inhibitor can be encapsulated within the lipid bilayer via passive loading first, and subsequently another water-soluble (hydrophilic) protein kinase inhibitor is actively loaded inside the aqueous core of the liposome. The current example describes the loading of dasatinib (DAS, poorly water-soluble) and afatinib (AFA, water-soluble) into the liposome based on the sequential passive and active loading method. The detailed preparation process is provided in the Experimental Methods (Sequential Passive and Active Drug Loading Method). The result in Table 8 shows the physicochemical properties of the resulting AFA/DAS liposomes. The liposome composition is based on DSPG/Cholesterol/DSPC. The DAS to AFA molar ratio in the final drug loaded liposomes was 3.0. The sample was stored at 2-8° C. to monitor short-term stability studies and the result is shown in FIG. 10. The data reflects that the AFA/DAS-L liposome product was physically stable at refrigerated condition.

TABLE 8
Physicochemical properties of the DSPG liposomes of AFA-L, DAS-L,
and AFA/DAS-L

| | | Mean Particle Size (nm)/ | AFA:DAS |
|---|---|---|---|
| Sample Name | Trapping Agent | PDI | Molar Ratio |
| AFA-L | TEA-SBE-β-CD | 134/0.043 | — |
| DAS-L | — | 134/0.193 | — |
| AFA/DAS-L | TEA-SBE-β-CD | 130/0.213 | 1:3 |

### Example 12

### Preparation and Characterization of Dasatinib and Ceritinib DSPG Co-Loaded Liposomes

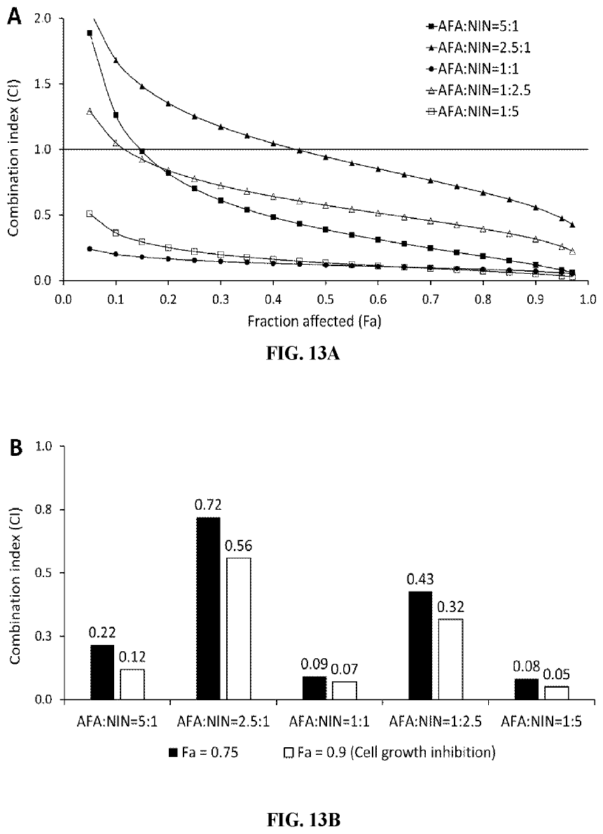

As illustrated from Scenario III of dual-drug encapsulation shown in FIG. 5 (III), two poorly water-soluble (lipophilic) protein kinase inhibitors can be co-loaded into the lipid bilayer of the liposome through a passive drug loading approach. The current example describes the encapsulation of ceritinib (CER) and dasatinib (DAS) into the liposome based on the procedure mentioned in the Experimental Methods (Passive Drug Loading Method). Physicochemical properties of the DAS/CER co-loaded liposome are shown in Table 9. The final ratio of encapsulated DAS to encapsulated CER in the co-loaded liposome was 1.8:1 (mol:mol). The sample was stored in a refrigerator to monitor short-term stability studies. As shown in FIG. 11, DAS/CER co-loaded liposome was physically stable at 2-8° C. within the studied time frame.

TABLE 9
Physicochemical properties of the DSPG liposomes co-loaded with
DAS/CER

| | | Molar Ratio |
|---|---|---|
| Sample Name | Mean Particle Size (nm)/PDI | DAS:CER |
| CER-L | 156/0.152 | n/a |
| DAS-L | 124/0.217 | n/a |
| DAS/CER-L | 105/0.193 | 1.8:1 |

CryoTEM was used to observe the morphology of the DSPG liposome co-loaded with DAS/CER at a molar drug ratio of 1.8 to 1. As shown in FIG. 8C, liposomes with unilamellar structures were observed, and the central aqueous core of the liposome was empty. The results indicate that both of the poorly water-soluble compounds (DAS and CER) were encapsulated within the lipid layer of the liposome.

### Example 13

### In-Vitro Evaluation on the Combination of Afatinib and Nintedanib for Synergy in Cancer Cells

For drug combination regimens, the two or more combined drugs could exhibit synergistic, additive, or antagonistic interaction. To identify the molar ratios of afatinib and nintedanib (AFA/NIN) that are synergistic, various drug-to-drug ratios of AFA/NIN were tested for their cytotoxic effects in cancer cell lines in-vitro. Measurement of the cytotoxic effects was performed using AFA/NIN at 10:1, 5:1, 2.5:1, 1:1, 1:2.5, 1:5 and 1:10 molar ratio in the following cancer cell lines: HT-29 colorectal cancer, A-549 non-small cell line cancer (NSCLC), MCF-7 breast cancer, H1975 NSCLC and HCC827 NSCLC. Cytotoxic effect from the treatment of AFA alone and NIN alone in the corresponding cell line were included as controls.

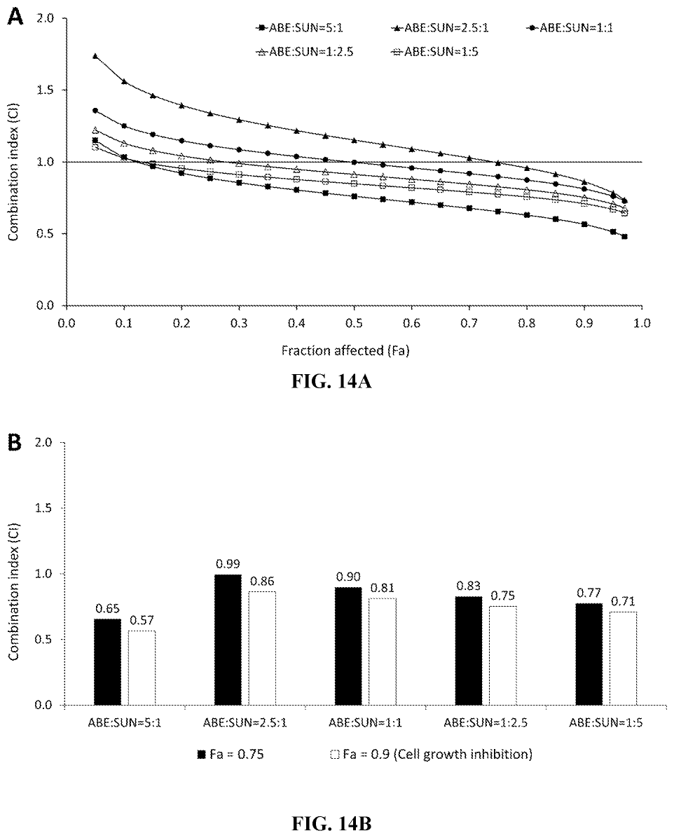

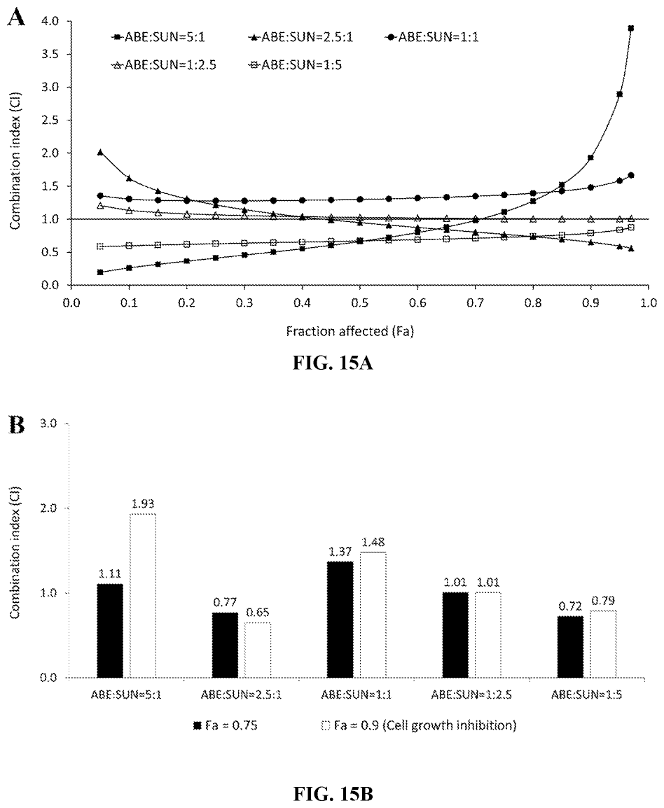

The detailed procedure on cell culture, drug treatment, MTT assay for cytotoxicity measurement were described in the Experimental Methods section (In vitro Cytotoxicity Study for Drug Synergy Determination). Based on the in-vitro efficacy results, a combination index was then determined for each Afatinib/Nintedanib dose using a software CompuSyn which is based on Chou and Talalay's theory of dose-effect analysis. In this theory, a “median-effect equation’ was used to calculate a number of biochemical equations that are extensively used in the art. Derivations of this equation have given rise to higher order equations such as those used to calculate Combination Index (CI). As mentioned previously, CI can be used to determine if combinations of more than one drug at various ratios are antagonistic (CI>1.1), additive (0.9≤CI≤1.1), or synergistic (CI<0.9). Table 10 shows the synergistic AFA/NIN molar ratio identified by the CI method for each cancer cell line tested. Representative plot based on CI values as a function of cell growth inhibition (%) of the combination of AFA/NIN evaluated in HT-29 colorectal cancer and H1975 NSCLC cells are shown in FIG. 12 and FIG. 13, respectively. Those synergistic drug ratios were then used for liposome drug product formulation to target the specific cancer cells.

TABLE 10
Synergistic drug molar ratios identified for the combination of
afatinib and nintedanib on various types of cancer cell lines

| | | Synergistic Drug Molar Ratio |
|---|---|---|
| Drug Combination | Cancer Cell Line | (AFA:NIN) |
| AFA + NIN | HT-29 colorectal | 2.5:1, 1:1, 1:2.5 and 1:5 |
| | A-549 NSCLC | 1:1 and 1:5 |
| | MCF-7 breast cancer | 1:1 |
| | H1975 NSCLC | 5:1, 1:1, 1:2.5 and 1:5 |
| | HCC827 NSCLC | 1:2.5 and 1:5 |

### Example 14

### In-Vitro Evaluation on the Combination of Abemaciclib and Sunitinib for Synergy in Cancer Cells

To identify the molar ratios of abemaciclib and sunitinib (ABE/SUN) that are synergistic, various drug-to-drug ratios of ABE/SUN were tested for their cytotoxic effects in cancer cell lines in-vitro. Measurement of the cytotoxic effects was performed using ABE/SUN at 10:1, 5:1, 2.5:1, 1:1, 1:2.5, 1:5 and 1:10 molar ratio in the following cancer cell lines: HT-29 colorectal cancer, A-549 non-small cell line cancer (NSCLC), 786-O renal cell carcinoma (RCC) and Caki-1 RCC. Cytotoxic effects from the treatment of ABE alone and SUN alone in the corresponding cell line were included as controls.

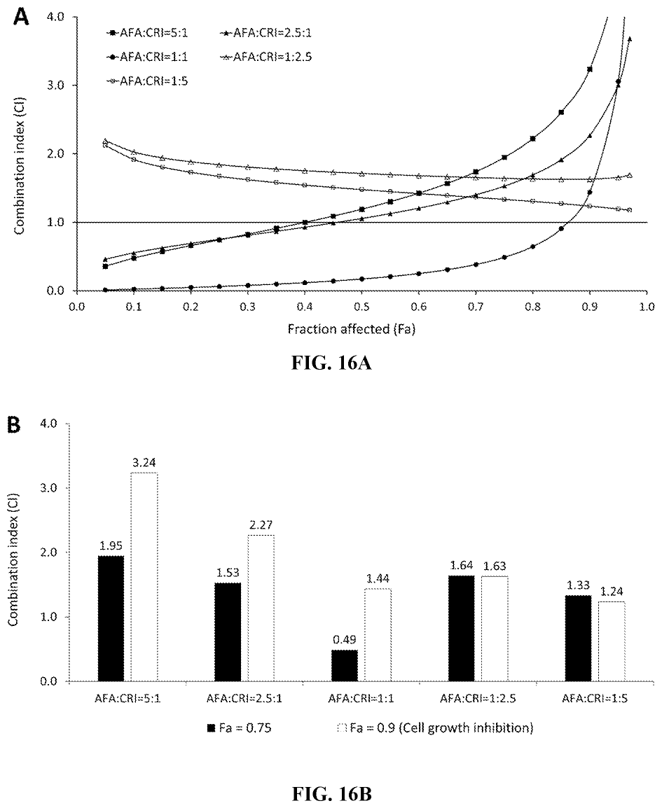

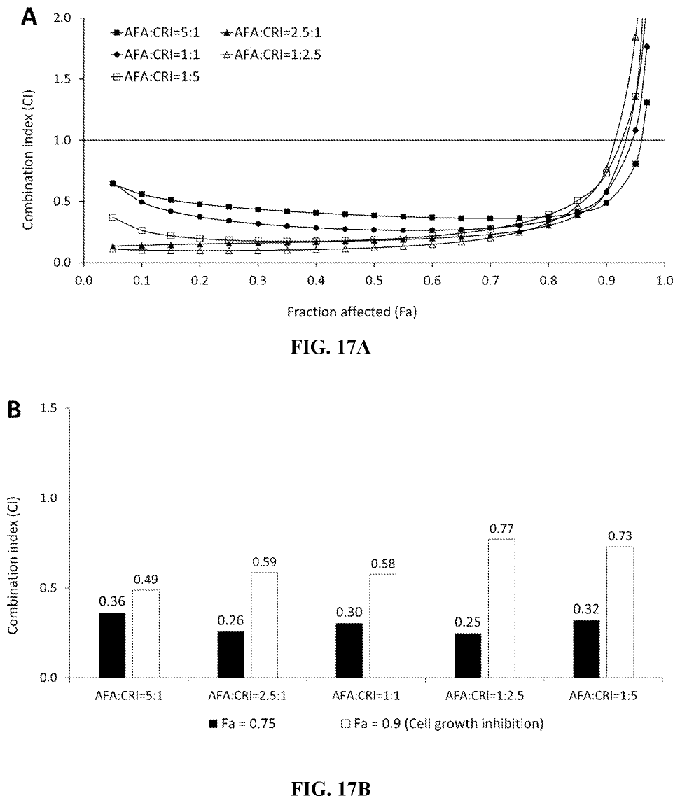

The detailed procedure on cell culture, drug treatment, MTT assay for cytotoxicity measurement and CI value calculation were described in the Experimental Method section (In vitro Cytotoxicity Study for Drug Synergy Determination). Table 11 shows the synergistic ABE/SUN molar ratio identified by the CI method for each cancer cell line tested. Representative plot based on CI values as a function of cell growth inhibition (%) of the combination of ABE/SUN evaluated in 786-O RCC and Caki-1 RCC are shown in FIG. 14 and FIG. 15, respectively. Those identified synergistic drug ratios were then used for liposome drug product formulation to target the specific cancer cells.

TABLE 11
Synergistic drug molar ratios identified for the combination of
abemaciclib and sunitinib on various types of cancer cell lines

| | | Synergistic Drug Molar Ratio |
|---|---|---|
| Drug Combination | Cancer Cell Line | (ABE:SUN) |
| ABE + SUN | HT-29 colorectal | 1:5 |
| | A-549 NSCLC | 1:5 and 1:10 |
| | 786-O RCC | 5:1 |
| | Caki-1 RCC | 1:5 |

### Example 15

### In-Vitro Evaluation on the Combination of Afatinib and Crizotinib for Synergy in Cancer Cells

To identify the molar ratios of afatinib and crizotinib (AFA/CRI) that are synergistic, various drug-to-drug ratios of AFA/CRI were tested for their cytotoxic effects in cancer cell lines in vitro. Measurement of the cytotoxic effects was performed using AFA/CRI at 10:1, 5:1, 2.5:1, 1:1, 1:2.5, 1:5 and 1:10 molar ratio in the following cancer cell lines: MSTO-211H mesothelioma and H1975 NSCLC. Cytotoxic effect from the treatment of AFA alone and CRI alone in the corresponding cell line were included as controls.

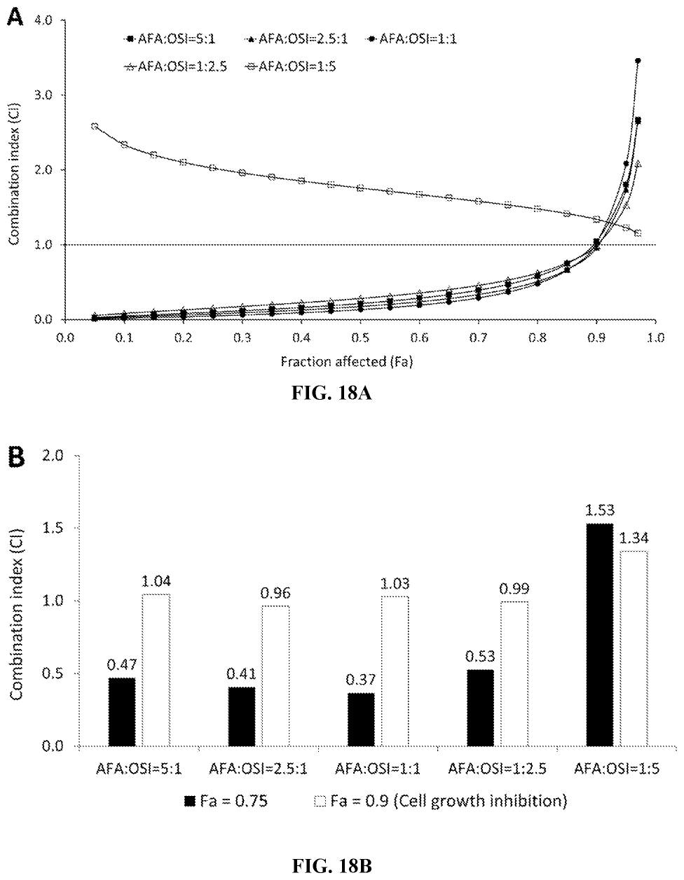

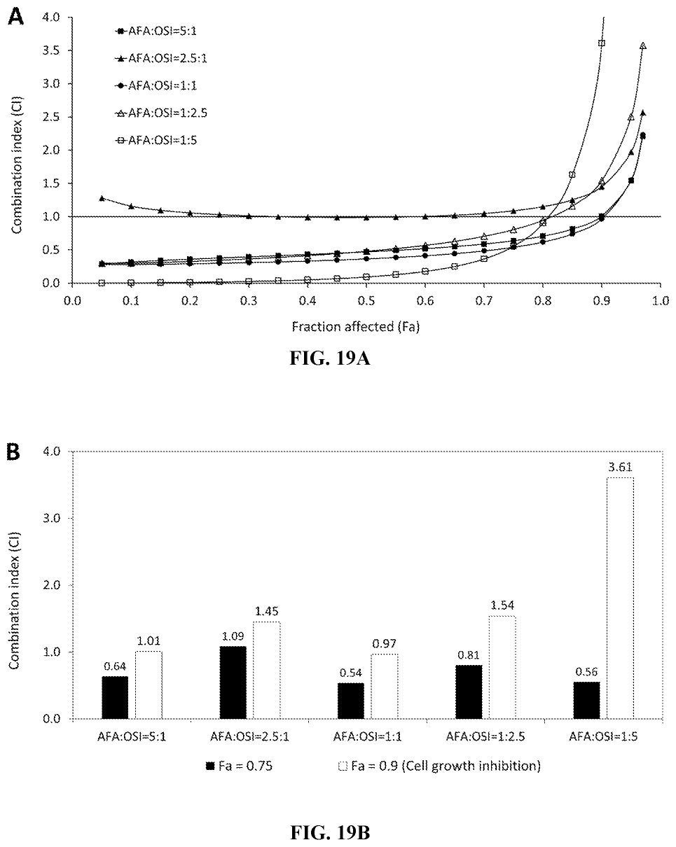

The detailed procedures for cell culture, drug treatment, MTT assay for cytotoxicity measurement and CI value calculation were described in the Experimental Method section (In vitro Cytotoxicity Study for Drug Synergy Determination). Table 12 shows the synergistic AFA/CRI molar ratio identified by the CI method for each cancer cell line tested. Representative plot based on CI values as a function of cell growth inhibition (%) of the combination of AFA/CRI evaluated in MSTO-211H mesothelioma and H1975 NSCLC cell lines are shown in FIG. 16 and FIG. 17, respectively. Those identified synergistic drug ratios were then used for liposome drug product formulation to target the specific cancer cells.

TABLE 12
Synergistic drug molar ratios identified for the combination afatinib
and crizotinib on various types of cancer cell lines

| | | Synergistic Drug Molar Ratio |
|---|---|---|
| Drug Combination | Cancer Cell Line | (AFA:CRI) |
| AFA + CRI | MSTO-211H | 1:1 |
| | mesothelioma | |
| | H1975 NSCLC | 5:1, 2.5:1 and 1:1 |

### Example 16

### In-Vitro Evaluation on the Combination of Osimertinib and Afatinib for Synergy in Cancer Cells

To identify the molar ratios of osimertinib and afatinib (OSI/AFA) that are synergistic, various drug-to-drug ratios of OSI/AFA were tested for their cytotoxic effects in cancer cell lines in vitro. Measurement of the cytotoxic effects was performed using OSI/AFA at 10:1, 5:1, 2.5:1, 1:1, 1:2.5, 1:5 and 1:10 molar ratio in the following cancer cell lines: H1975 NSCLC and HCC827 NSCLC. Cytotoxic effect from the treatment of OSI alone and AFA alone in the corresponding cell line were included as controls.

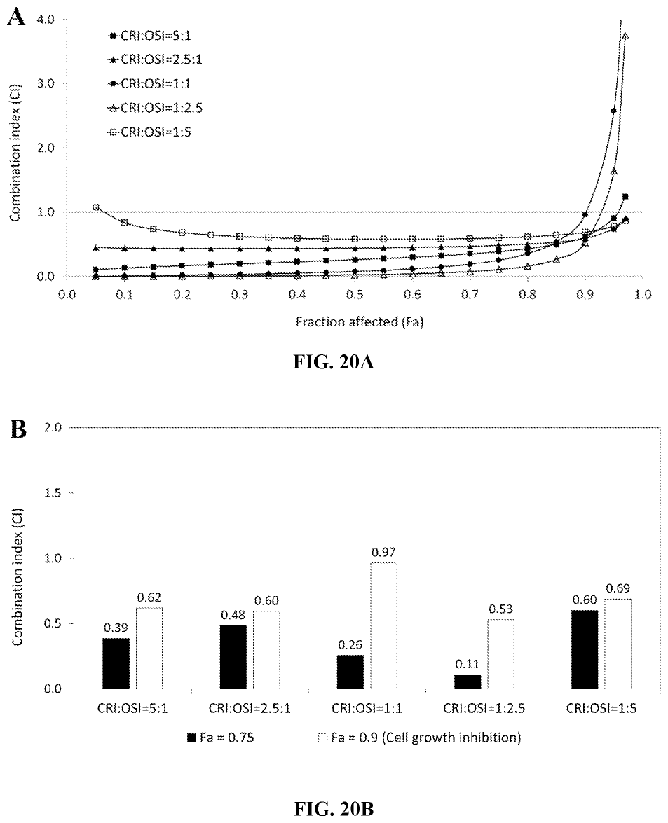

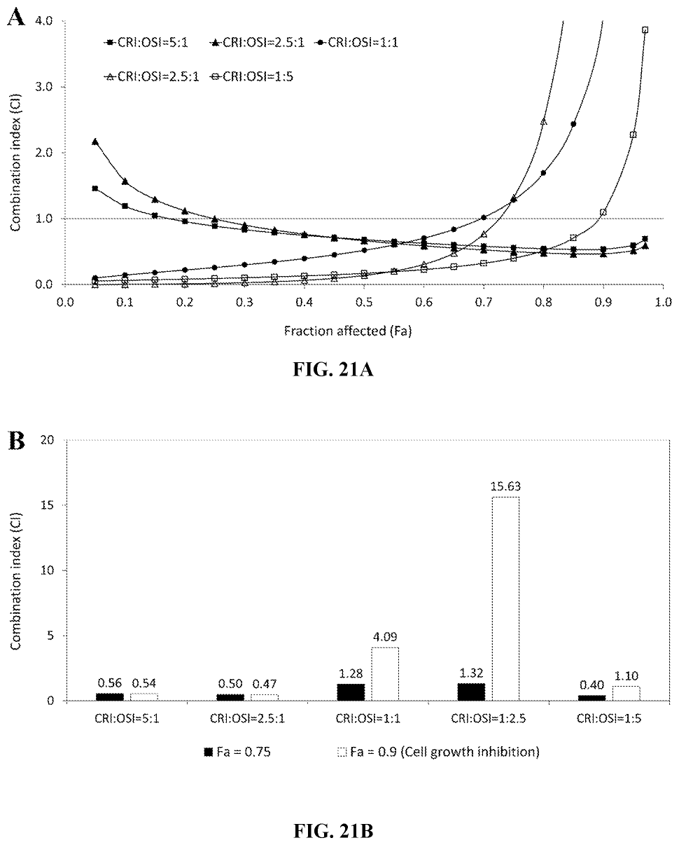

The detailed procedures for cell culture, drug treatment, MTT assay for cytotoxicity measurement and CI value calculation were described in the Experimental Method section (In vitro Cytotoxicity Study for Drug Synergy Determination). Table 13 shows the synergistic OSI/AFA molar ratio identified by the CI method for each cancer cell line tested. Representative plot based on CI values as a function of cell growth inhibition (%) of the combination of OSI/AFA evaluated in H1975 NSCLC and HCC827 cell lines are shown in FIG. 18 and FIG. 19, respectively. Those identified synergistic drug ratios were then used for liposome drug product formulation to target the specific cancer cells.

TABLE 13
Synergistic drug molar ratios identified for the combination osimertinib
and afatinib on various types of cancer cell lines

| | | Synergistic Drug Molar Ratio |
|---|---|---|
| Drug Combination | Cancer Cell Line | (OSI:AFA) |
| OSI + AFA | H1975 NSCLC | 5:1, 2.5:1, 1:1 and 2.5:1 |
| | HCC827 NSCLC | 5:1 and 1:1 |

### Example 17

### In-Vitro Evaluation on the Combination of Osimertinib and Crizotinib for Synergy in Cancer Cells

To identify the molar ratios of osimertinib and crizotinib (OSI/CRI) that are synergistic, various drug-to-drug ratios of OSI/CRI were tested for their cytotoxic effects in cancer cell lines in vitro. Measurement of the cytotoxic effects was performed using OSI/CRI at 10:1, 5:1, 2.5:1, 1:1, 1:2.5, 1:5 and 1:10 molar ratio in the following cancer cell lines: H1975 NSCLC and HCC827 NSCLC. Cytotoxic effect from the treatment of OSI alone and CRI alone in the corresponding cell line were included as controls.

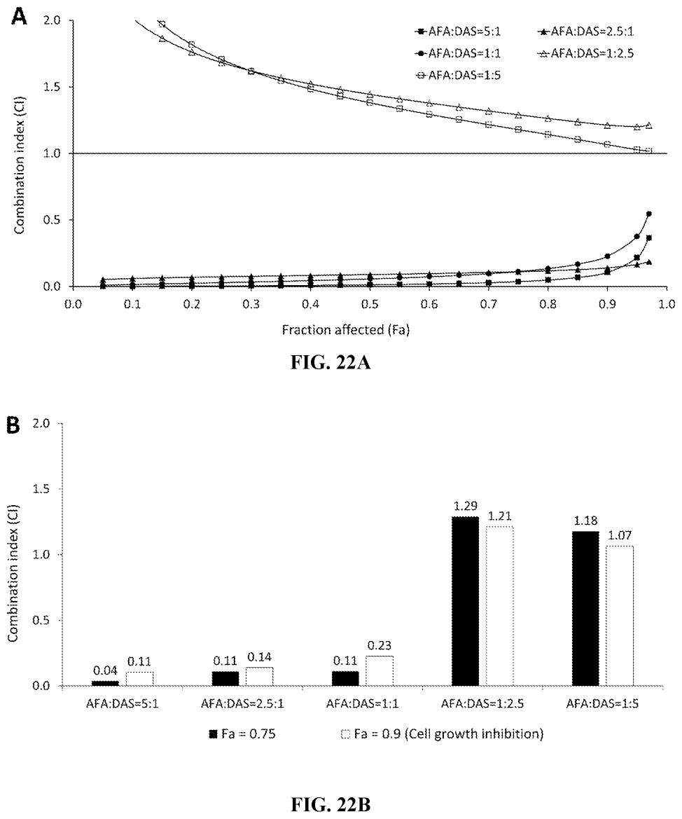

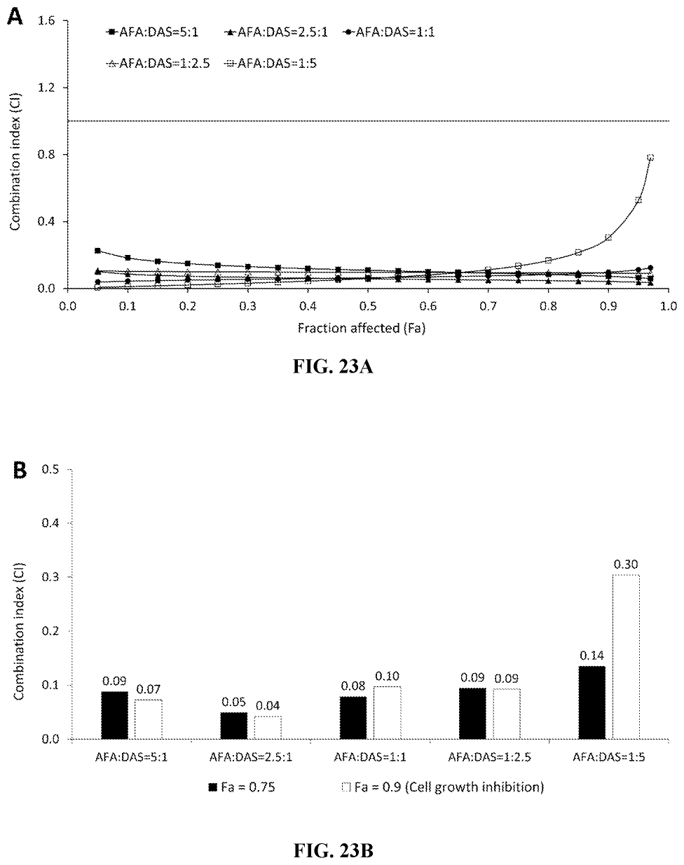

The detailed procedures for cell culture, drug treatment, MTT assay for cytotoxicity measurement and CI value calculation were described in the Experimental Method section (In vitro Cytotoxicity Study for Drug Synergy Determination). Table 14 shows the synergistic OSI/CRI molar ratio identified by the CI method for each cancer cell line tested. Representative plot based on CI values as a function of cell growth inhibition (%) of the combination of OSI/CRI evaluated in H1975 NSCLC and HCC827 cell lines are shown in FIG. 20 and FIG. 21, respectively. Those identified synergistic drug ratios were then used for liposome drug product formulation to target the specific cancer cells.

TABLE 14
Synergistic drug molar ratios identified for the combination osimertinib
and crizotinib on various types of cancer cell lines

| | | Synergistic Drug Molar Ratio |
|---|---|---|
| Drug Combination | Cancer Cell Line | (OSI:CRI) |
| OSI + CRI | H1975 NSCLC | 5:1 and 1:2.5 |
| | HCC827 NSCLC | 1:5 and 1:2.5 |

### Example 18

### In-Vitro Evaluation on the Combination of Afatinib and Dasatinib for Synergy in Cancer Cells

To identify the molar ratios of afatinib and dasatinib (AFA/DAS) that are synergistic, various drug-to-drug ratios of AFA/DAS were tested for their cytotoxic effects in cancer cell lines in vitro. Measurement of the cytotoxic effects was performed using AFA/DAS at 10:1, 5:1, 2.5:1, 1:1, 1:2.5, 1:5 and 1:10 molar ratio in the following cancer cell lines: MSTO-211H mesothelioma, H1975 NSCLC and HCC827 NSCLC. Cytotoxic effect from the treatment of AFA alone and DAS alone in the corresponding cell line were included as controls.

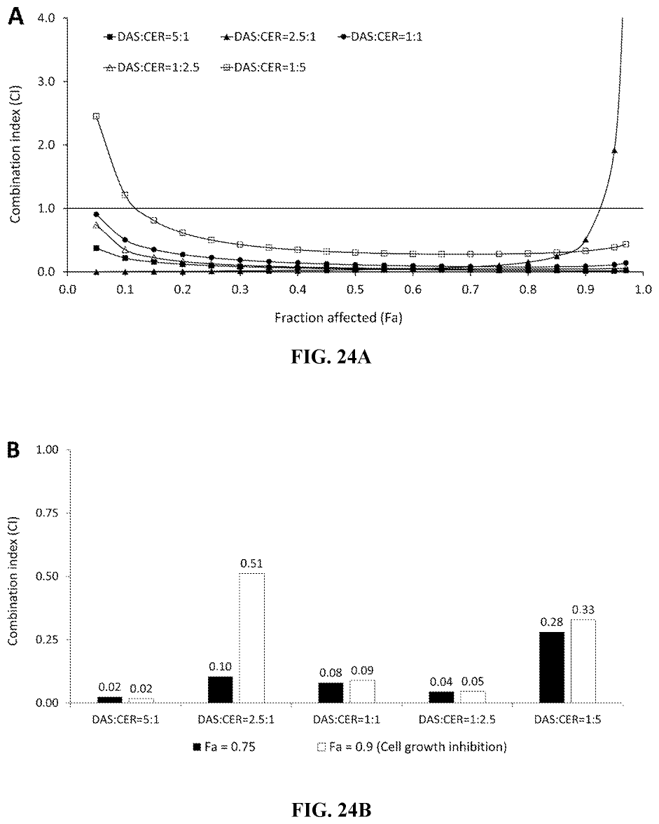

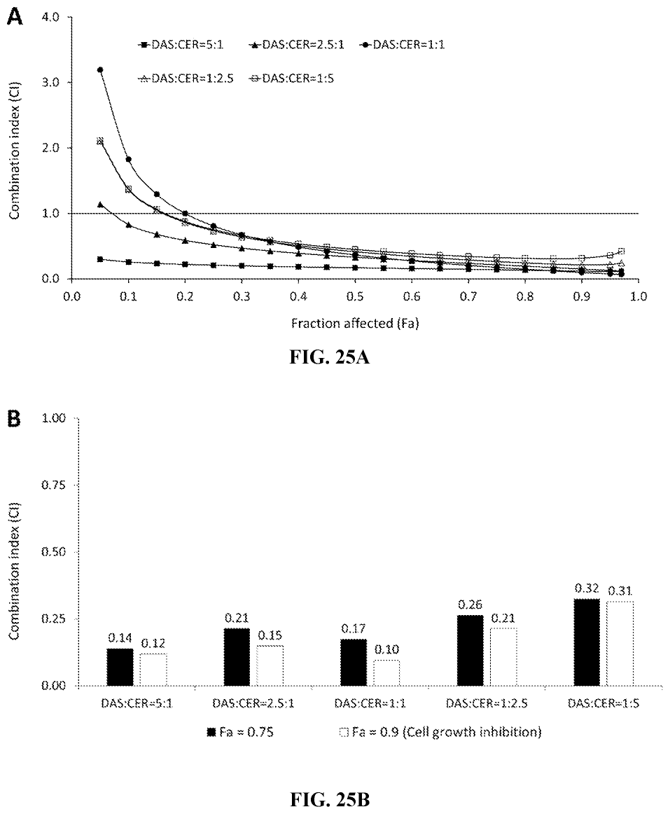

The detailed procedures for cell culture, drug treatment, MTT assay for cytotoxicity measurement and CI value calculation were described in the Experimental Method section (In vitro Cytotoxicity Study for Drug Synergy Determination). Table 15 shows the synergistic AFA/DAS molar ratio identified by the CI method for each cancer cell line tested. Representative plot based on CI values as a function of cell growth inhibition (%) of the combination of AFA/DAS evaluated in H1975 NSCLC and HCC827 cell lines are shown in FIG. 22 and FIG. 23, respectively. Those identified synergistic drug ratios were then used for liposome drug product formulation to target the specific cancer cells.

TABLE 15
Synergistic drug molar ratios identified for the combination afatinib
and dasatinib on various types of cancer cell lines

| | | Synergistic Drug Molar Ratio |
|---|---|---|
| Drug Combination | Cancer Cell Line | (AFA:DAS) |
| AFA + DAS | MSTO-211H | 1:1, 2.5:1 and 5:1 |
| | mesothelioma | |
| | H1975 NSCLC | 5:1 and 1:2.5 |
| | HCC827 NSCLC | 1:5 and 1:2.5 |

### Example 19

### In-Vitro Evaluation on the Combination of Dasatinib and Ceritinib for Synergy in Cancer Cells

To identify the molar ratios of dasatinib and ceritinib (DAS/CER) that are synergistic, various drug-to-drug ratios of DAS/CER were tested for their cytotoxic effects in cancer cell lines in vitro. Measurement of the cytotoxic effects was performed using DAS/CER at 10:1, 5:1, 2.5:1, 1:1, 1:2.5, 1:5 and 1:10 molar ratio in the following cancer cell lines: H1975 NSCLC and HCC827 NSCLC. Cytotoxic effect from the treatment of DAS alone and CER alone in the corresponding cell line were included as controls.

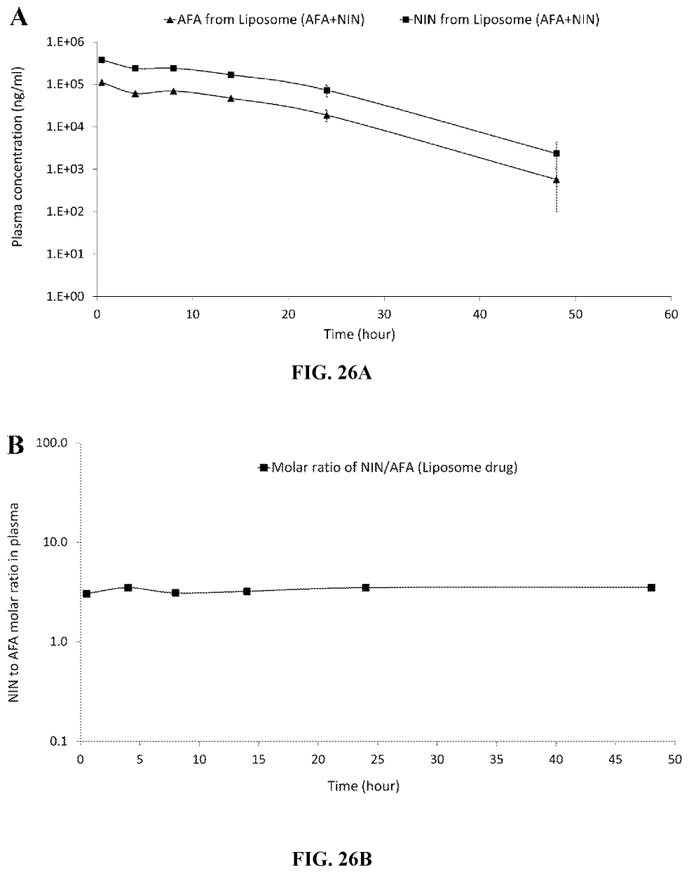

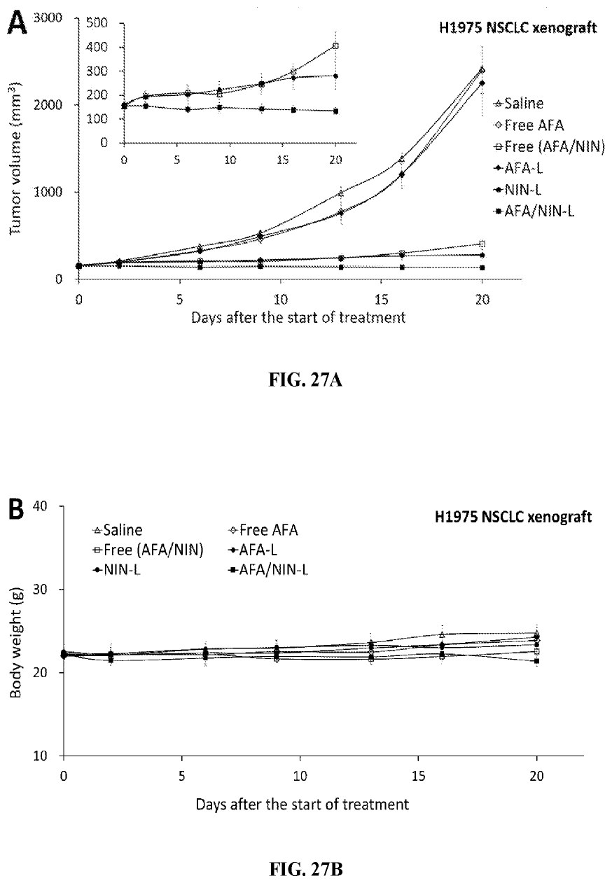

The detailed procedures for cell culture, drug treatment, MTT assay for cytotoxicity measurement and CI value calculation were described in the Experimental Method section (In vitro Cytotoxicity Study for Drug Synergy Determination). Table 16 shows the synergistic DAS/CER molar ratio identified by the CI method for each cancer cell line tested. Representative plot based on CI values as a function of cell growth inhibition (%) of the combination of DAS/CER evaluated in H1975 NSCLC and HCC827 cell lines are shown in FIG. 24 and FIG. 25, respectively. Those identified synergistic drug ratios were then used for liposome drug product formulation to target the specific cancer cells.

TABLE 16
Synergistic drug molar ratios identified for the combination dasatinib
and ceritinib on various types of cancer cell lines

| | | Synergistic Drug Molar Ratios |
|---|---|---|
| Drug Combination | Cancer Cell Line | (DAS:CER) |
| DAS + CER | H1975 NSCLC | 5:1, 1:1, 1:2.5, 1:5 |
| | HCC827 NSCLC | 5:1, 2.5:1 |

### Example 20

### In-Vitro Tumor Cell Growth Inhibition by Afatinib/Nintedanib Co-Loaded Liposomes

The cancer cell growth inhibition by both the liposome co-loaded with two kinase inhibitors (e.g., AFA/NIN-L), and the corresponding single drug loaded liposomes (e.g., AFA-L and NIN-L) were studied in vitro. All drug-loaded liposomes used in this study were PEGylated, and TEA-SOS was used as the trapping agent for all the drug products.

A brief experimental procedure is stated as follows: cancer cells were seeded onto the 96-well plate with appropriate seeding density. The cell seeded plate was incubated for 24 hours at 37° C. and 5% CO₂ in a standard cell culture incubator before the drug treatment. The following day, serial dilutions on liposome drug product were prepared using respective cell culture media. The cell culture media in the 96-well plate was then replaced by fresh media containing liposomal drugs. After a total of 48 hours of incubation, cell viability was assessed by the MTT assay following the manufacture's protocols. Relative percent survival was determined by subtracting absorbance values obtained by media-only wells from drug treated wells and then normalizing to the untreated control wells (cell only control). The percentage of cell growth inhibition is calculated by subtracting the % of cell viability from 100%.

The cell growth inhibition for AFA/NIN liposome drug product is shown in Table 17. Overall, the cell growth inhibition capability of the dual drug loaded liposome at their synergistic ratio was significantly greater than what would be expected if each encapsulated drug had contributed in only an additive fashion to its activity. Specifically, the cell growth inhibition was 59% for AFA/NIN-L at the total drug concentration of 2.34 μM. In contrast, the corresponding cell growth inhibition for AFA-L and NIN-L were 37% and 3% at the concentration at 0.39 μM and 1.95 μM respectively, which gives a total inhibition of only 40% based on the additive calculation. Therefore, there is a 50% increase on efficacy by the drug combination approach. Overall, the above result demonstrated that the synergistic effect based on the free drug combination of AFA/NIN shown in Example 13 can be well translated into the dual drug loaded liposome when the same drug ratio was employed.

TABLE 17
In-vitro cell growth inhibition on H1975 NSCLC cells by AFA/NIN
co-loaded liposomes (AFA/NIN-L)

| Sample Name | Cell line | Dose | Cell growth inhibition (%) |
|---|---|---|---|
| AFA-L | H1975 NSCLC | 0.39 μM | 37% |
| NIN-L | | 1.95 μM | 3% |
| AFA/NIN-L | | 2.34 μM | 59% |
| (1:5 molar ratio) | | | |

### Example 21

### In-Vitro Tumor Cell Growth Inhibition by Afatinib/Dasatinib Co-Loaded Liposomes

The cancer cell growth inhibition by both the liposome co-loaded with two kinase inhibitors (e.g., AFA/DAS-L), and the corresponding single drug loaded liposomes (e.g., AFA-L and DAS-L) were studied in-vitro. All drug-loaded liposomes used in this study were DSPG liposomes, and TEA-SBE-β-CD was used as the trapping agent.

The method on cell culture, liposome drug product treatment and the quantification of cell growth inhibition by MTT assay are mentioned in Example 20. The cell growth inhibition for AFA/DAS liposome drug product is shown in Table 18. Overall, the cell growth inhibition capability of the dual drug loaded liposome at their synergistic ratio was significantly greater than what would be expected if each encapsulated drug had contributed in only an additive fashion to its activity. Specifically, the cell growth inhibition was 88% for AFA/DAS-L at the total drug concentration of 34 nM. In contrast, the corresponding cell growth inhibition for AFA-L and DAS-L were 3% and 33% at the concentration at 24 nM and 10 nM respectively, which gives a total inhibition of only 36% based on the additive calculation. Therefore, there is a 2.4 fold of increase on efficacy by the drug combination approach. Overall, the above result demonstrated that the synergistic effect based on the free drug combination of AFA/DAS shown in Example 18 can be well translated into the dual drug loaded liposome formulation when the same drug ratio was employed.

TABLE 18
In-vitro cell growth inhibition on HCC827 NSCLC cells by co-loaded
liposomes (AFA/DAS-L)

| Sample name | Cell line | Dose | Cell growth inhibition (%) |
|---|---|---|---|
| AFA-L | HCC827 NSCLC | 24 nM | 3% |
| DAS-L | | 10 nM | 33% |
| AFA/DAS-L | | 34 nM | 88% |
| (2.6:1 molar ratio) | | | |

### Example 22

### In-Vivo Pharmacokinetic Study on Co-Loaded PEGylated AFA/NIN Liposome

In-vivo pharmacokinetic study of free afatinib and nintedanib combination (data not shown), and AFA/NIN-L (1:5 AFA:NIN molar ratio) were carried out in BALB/c nude mice. The preparation of the PEGylated AFA/NIN-L with TEA-SOS used as the trapping agent was described in the Experimental Method section. The mixture of afatinib and nintedanib free drug cocktail and PEGylated AFA/NIN-L with a molar ratio of 1:5 were administered intravenously via the tail vein into the mice and the plasma concentration of both AFA and NIN was monitored over time. Doses of the all drug formulations were 7.0 mg/kg of afatinib free base (10.3 mg/kg afatinib dimaleate) and 38.9 mg/kg of nintedanib free base (46.8 mg/kg nintedanib mesylate). After intravenous administration, blood was collected at multiple time points by cardiac puncture (3 mice per time point) and placed into EDTA coated micro containers. The samples were centrifuged to separate plasma, and plasma was transferred to another tube. Then, AFA and NIN plasma levels were quantified with LC-MS. PK parameters were then calculated from measured AFA and NIN plasma levels.

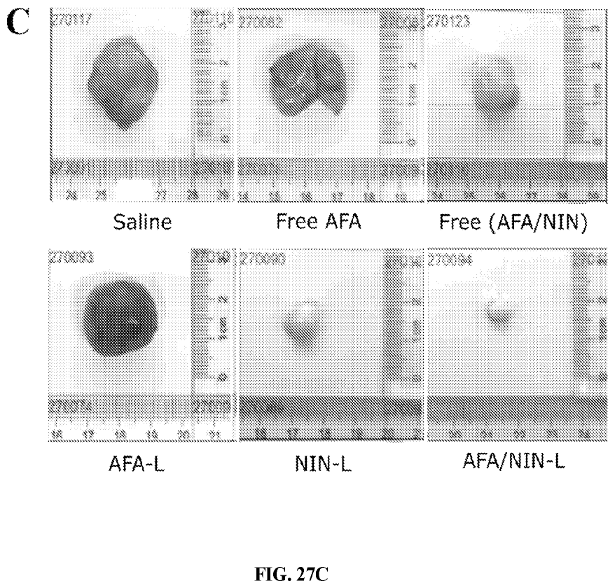

The pK study results revealed that the free AFA and free NIN drug solutions were rapidly eliminated after IV administration, and the half-life (TI/2) value of the free AFA and free NIN was very difficult to be determined. Comparing to the free drug solutions, PK profile of AFA and NIN in the PEGylated AFA/NIN-L demonstrated a prolonged circulation time (FIG. 26A). The AUC (Area Under Curve) of both drugs in AFA/NIN-L liposome was increased >700 (AFA) and >350 (NIN) fold higher than those in the free drug solutions. The MRT (Maximum Retention Time) of both drugs in PEGylated AFA/NIN-L liposome was increased >4.5 (AFA) and 6 (NIN) fold higher than those in the free drug solutions too. These PK results demonstrated the co-loaded AFA/NIN-L liposome significantly extended the drug retention and exhibits substantially increased plasma drug levels of AFA and NIN in mice comparing to the free drug of AFA and NIN. Furthermore, it was noticed that the AFA/NIN-L liposome drug product was well tolerated at the above-mentioned drug dose by the mice and no adverse events were observed in this PK study.

In addition, the molar ratio of AFA and NIN in plasma could also be determined at different time points. FIG. 26B shows the molar ratio of AFA/NIN in plasma over a total of 48 hours after intravenous administration to mice. The result reflects that the molar ratio of AFA and NIN from the liposome formulation was very well maintained at its initial feed level over the course of 48 hours. In contrast, large deviation of drug molar ratio from initial feed (AFA:NIN=1:5 by molar) was observed for the free drug cocktail group (data not shown). As discussed in Example 13, a molar ratio of AFA:NIN maintained in a range 5:1 to 1:5 exhibit a synergistic cell growth inhibition effect in both HT-29 and H-1975 cancer cell lines. Therefore, the result from PK study demonstrated that the appropriately designed liposome formulation can maintain the synergistic drug molar ratio over a prolonged period of time. Moreover, the LC-MS analysis indicated that circulating liposomal drug formulations contained intact drug and no evidence of the degradation and cross interaction between the afatinib and nintedanib was observed for either compound.

### Example 23

### In-Vivo Tumor Regression in Mice Bearing H1975 NSCLC Xenograft Tumors

The potential of AFA/NIN-L to suppress NSCLC cancer was evaluated in mice bearing H1975 xenograft tumors. To maximize the therapeutic activity of drug combinations and to capture the synergistic benefits observed in-vitro cell line studies, the drug combination needs to be delivered to the tumors site at the synergistic drug to drug ratio. For this purpose, the liposome formulations containing AFA and NIN at the fixed ratio known to be synergistic in H1975 NSCLC cells were developed (Example 4). The antitumor activity of this formulation was then evaluated in H1975 NSCLC model in-vivo. The PEGylated liposomes co-encapsulated with AFA and NIN at a synergistic molar ratio 1:5 with TEA-SOS as the trapping agent was used for this study.

Tumor cell inoculation and tumor xenograft establishment was mentioned in the Experimental Methods section (In-vivo Efficacy Study). Mice were organized into appropriate treatment groups consisting of saline control and drug treated groups including (1) Free AFA, (2) AFA-L, (3) NIN-L, (4) AFA/NIN mixed free drug cocktail solution (AFA:NIN molar ratio of 1:5), and (5) AFA/NIN-L (AFA:NIN molar ratio of 1:5). Mice were injected intravenously with the required volume of sample to administer the targeted dose (7.0 mg/kg AFA free base, 38.9 mg/kg NIN free base) to the animals based on the weight of each individual mice every two days for a total of 27 days. The tumor volume and mice weight are measured and monitored over time.

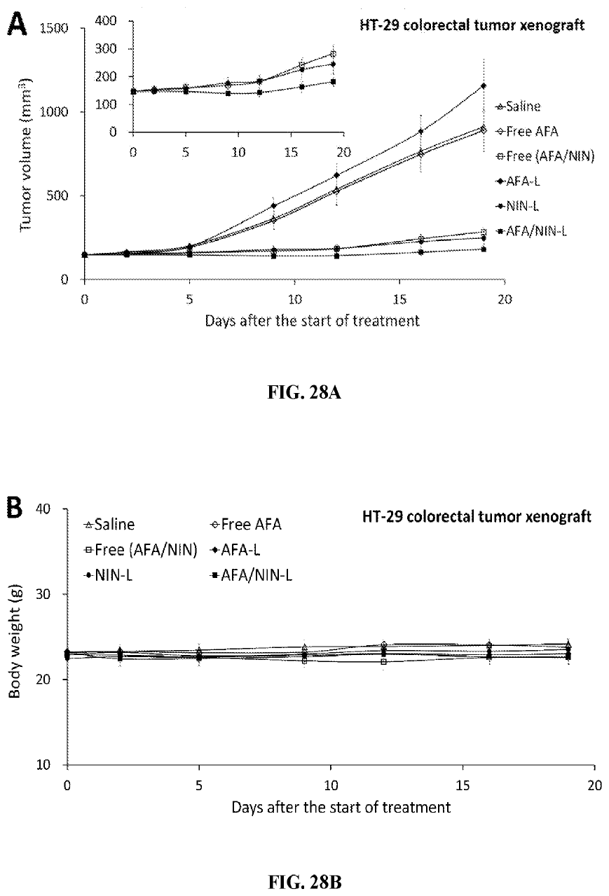

As shown in FIG. 27A, in general, the co-loaded liposome (AFA/NIN-L) significantly restrained tumor growth after 27 days of treatment as compared to those observed in the tumor bearing saline control group as well as other drug treated groups including free AFA drug, AFA-L monotherapy, NIN-L monotherapy and combined free AFA/NIN cocktail solution. For the control, free-AFA, and AFA-L groups, the average tumor size (n=6) increased dramatically (>15 fold) after the full course of treatment. This implies the free afatinib does not have any inhibition in this specific cell line which could be due to its resistance to afatinib. For the free AFA/NIN combo cocktail and NIN-L groups, the average tumor sizes increased about 170% and 47% after 20 days of treatment, respectively. In contrast, a significant reduction on tumor volume was observed from mice treated by AFA/NIN-L, which corresponds to a 10% of size decrease after the same period of treatment.

In addition, all drug treatment groups did not cause significant reduction in mice body weight compared to that from control (FIG. 27B). Therefore, the results indicate there was no general systemic toxicity for all drug treated groups. However, it was observed that the injection site of mice tail was swollen and red for the group treated by the AFA/NIN free drug cocktail. In contrast, the liposome formulations (i.e., AFA-L, NIN-L and AFA/NIN-L) did not cause any damage on the injection site. Therefore, we can deduce that encapsulation by liposome shielded the mice from the toxicity associated with AFA and NIN at the injection site. Photos of dissected H1975 xenograft tumor masses from both control (untreated) and all drug treated mice are presented in FIG. 27C. Significant tumor size reduction was observed from mice treated by AFA/NIN free drug cocktail, NIN-L and AFA/NIN-L and the degree of such reduction was observed in the order of AFA/NIN-L>NIN-L>AFA/NIN free drug cocktail. In contrast, dramatically larger tumor size was observed from untreated control, free AFA, and AFA-L treated groups, and there was no significant tumor size difference among those three groups. These results indicate that AFA/NIN-L exhibits significant improved antitumor activity compared to other drug formulations.

Without being bound by theory, this suppression of the tumor growth by AFA/NIN-L is believed to be attributed to the enhanced permeability and retention (EPR) effect of the nanosized liposome formulation, which can improve the drug delivery specificity in the tumor site. Therefore, the comparison on the efficacy between free drug cocktail and liposomal drug combination reflects that the superior biodistribution achieved by the current liposome formulation can lead to more efficient tumor suppression. It is interesting to find that mice treated by AFA free drug and AFA-L monotherapy has similar tumor growth rate as observed in the saline control group, which implies the possible intrinsic afatinib resistant properties of H1975 NSCLC cell line. Also, the effect on tumor growth inhibition imposed by AFA/NIN combination was significantly stronger as compared to that from either NIN-L or AFA-L. The result indicates that the synergistic effect between AFA and NIN found from in-vitro studies (Example 13) can be well translated into the liposome formulation in the in-vivo settings. Overall, the above results demonstrated that fixing synergistic AFA to NIN molar ratio by encapsulating them inside liposomes can dramatically improve antitumor activity.

### Example 24

### In-Vivo Tumor Regression in Mice Bearing HT-29 Colorectal Cell Xenograft Tumors

As described in Example 15, the antitumor activity of the AFA/NIN co-loaded liposome formulation was also evaluated in tumor xenograft model in mice bearing HT-29 colorectal tumor. The PEGylated liposomes co-encapsulated with AFA and NIN at a synergistic molar ratio of 1:5 (AFA/NIN) with TEA-SOS as trapping agent was used for this study.

Tumor cell inoculation and tumor xenograft establishment was mentioned in the Experimental Methods section (In vivo Efficacy Study). Mice were organized into appropriate treatment groups consisting of saline control and drug treated groups including (1) Free AFA, (2) AFA-L, (3) NIN-L, (4) AFA/NIN mixed free drug cocktail solution (AFA:NIN molar ratio of 1:5), and (5) AFA/NIN-L (AFA:NIN molar ratio of 1:5). Mice were injected intravenously with the required volume of sample to administer the targeted dose (7.0 mg/kg AFA free base, 38.9 mg/kg NIN free base) to the animals based on the weight of each individual mice every two days for a total of four weeks. The tumor volume and mice weight are measured and monitored over time.

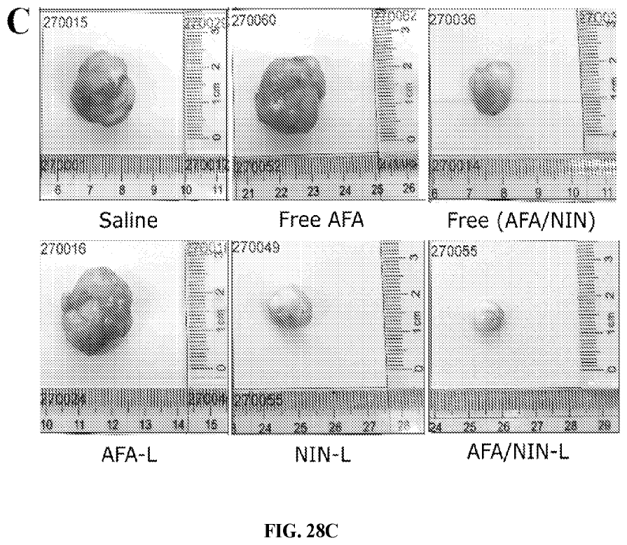

As shown in FIG. 28A, in general, the co-loaded liposome (AFA/NIN-L) significantly restrained tumor growth after 19 days of treatment as compared to those observed in the tumor bearing saline control group as well as other drug treated groups including free AFA drug, AFA-L monotherapy. For the control, free-AFA, and AFA-L groups, the average tumor size (n=6) increased dramatically (>6 fold) after 19 days of treatment. This implies the free afatinib does not have any inhibition in this specific cell line which could be due to its resistance to afatinib. For the free AFA/NIN combo cocktail and NIN-L groups, the average tumor sizes increased about 95% and 64% after the 19 days of treatment, respectively. In contrast, an average tumor size (n=6) was increased only 24% from AFA/NIN-L treated group by the same treatment period.

In addition, afatinib, as monotherapy and in combination with nintedanib in free drug or liposome form, did not cause significant reduction in body weight compared to that from control (FIG. 28B). Therefore, the results indicate there was no general systemic toxicity for all drug treated groups. However, similarly as described in H1975 NSCLC xenograft study (Example 23), it was observed that the injection site of mice tail was swollen and red for the group treated by the AFA/NIN free drug cocktail. In contrast, the AFA-L, NIN-L, and AFA/NIN-L did not cause any damage on the injection site. Therefore, we can deduce that the liposome formulation shielded the mice from the toxicity associated with AFA and NIN. Photos of dissected HT-29 xenograft tumor masses from both control (untreated) and all drug treated mice are presented in FIG. 28C. Significant tumor size reduction was observed from mice treated by AFA/NIN free drug cocktail, NIN-L and AFA/NIN-L and the degree of such reduction was observed in the order of AFA/NIN-L>NIN-L>AFA/NIN free drug cocktail. In contrast, dramatically larger tumor size was observed from untreated control, free AFA, and AFA-L treated groups, and there was no significant tumor size difference among those three groups. These results indicate that AFA/NIN-L exhibits significant improved antitumor activity compared to other drug formulations.

Without being bound by theory, this suppression of the tumor growth by AFA/NIN-L is believed to be attributed to the enhanced permeability and retention (EPR) effect of the nanosized liposome formulation, which can improve the drug delivery specificity in the tumor site. Therefore, the comparison on the efficacy between free drug cocktail and liposomal drug combination reflects that the superior biodistribution achieved by the current liposome formulation can lead to more efficient tumor suppression. It is interesting to find that mice treated by AFA free drug and AFA-L monotherapy has similar tumor growth rate as observed in the saline control group, which implies the possible intrinsic afatinib resistant properties of HT-29 colorectal cell line. Also, the effect on tumor growth inhibition imposed by AFA/NIN combination was significantly stronger as compared to that from either NIN-L or AFA-L. The result indicates that the synergistic effect between AFA and NIN found from in-vitro studies (Example 13) can be well translated into the liposome formulation in the in-vivo settings. Overall, the above results demonstrated that fixing synergistic AFA to NIN molar ratio by encapsulating them inside liposomes can dramatically improve antitumor activity.

The foregoing embodiments and examples are provided for illustrative purposes only and are not intended to limit the scope of the invention. Many variations to those described above may be possible. Since various modifications and variations to the embodiments and examples described above will be apparent to those of skill in this art based on the present disclosure, such modifications and variations are within the spirit and scope of the present invention. All patent or non-patent literature cited are incorporated herein by reference in their entireties without admission of them as prior art.

1. A pharmaceutical composition, comprising a liposome, one, two, or more protein kinase inhibitors, and a liquid medium, wherein the liposome comprises an aqueous interior core and a lipophilic exterior bilayer membrane, and wherein the bilayer membrane comprises an inner surface enclosing the interior core and an outer surface in contact with the liquid medium; and wherein one, two, or more protein kinase inhibitor(s) are encapsulated in either the aqueous interior core or the hydrophobic lipid bilayer membrane, or both; and wherein the two or more protein kinase inhibitors encapsulated inside of the liposome can be released from the liposome to function in a synergistic mode.

2. The pharmaceutical composition of claim 1, wherein the lipid bilayer membrane comprises (a) at least 10 mol % of a phospholipid selected from phosphatidylcholine, phosphatidylglycerol, phosphatidylinositol, glyceroglycolipids, sphingoglycolipids, and combinations thereof, (b) sterol; and (c) optionally a charged phospholipid derivatized to polyethylene glycol.

3. The pharmaceutical composition of claim 2, wherein the sterol comprises about 0-60 mole % of total lipids or a derivative thereof.

4. The pharmaceutical composition of claim 1, wherein the outer surface of the lipid bilayer membrane comprises a negative charged lipid or a surface-modifying agent containing polyethylene glycol, wherein the molar ratio of the total lipid to the total protein kinase inhibitors is ≥1:1.

5. The pharmaceutical composition of claim 1, wherein the molar ratio of the two kinase inhibitors is in the range from about 60:1 to about 1:60, optionally about 30:1 to about 1:30, or about 10:1 to about 1:10.

6. The pharmaceutical composition of claim 1, wherein the liposomes have a mean particle size between 4.5 nm to 450 nm, optionally between 25 nm and 300 nm, or between 50 nm to 200 nm.

7. The pharmaceutical composition of claim 1, wherein the protein kinase inhibitors are independently selected from acalabrutinib, abemaciclib, afatinib, aflibercept, alectinib, avapritinib, axitinib, baricitinib, brigatinib, binimetinib, bosutinib, cabozantinib, capmatinib, ceritinib, cobimetinib, crizotinib, dabrafenib, dacomitinib, dasatinib, encorafenib, entrectinib, erdafitinib, erlotinib, everolimus, fedratinib, fostamatinib, gefitinib, gilteritinib, ibrutinib, icotinib, imatinib, lapatinib, larotrectinib, lenvatinib, lorlatinib, midostaurin, neratinib, nilotinib, nintedanib, netarsudil, osimertinib, pacritinib, pazopanib, pexidartinib, pemigatinib, palbociclib, ponatinib, pexidartinib, ponatinib, pralsetinib, quizartinib, regorafenib, ribociclib, ripretinib, ruxolitinib, selpercatinib, selumetinib, sorafenib, sunitinib, temsirolimus, tofacitinib, trametinib, tucatinib, upadacitinib, vandetanib, vemurafenib, zanubrutinib, ziv-aflibercept, and combinations thereof.

8. The pharmaceutical composition of claim 1, wherein the protein kinase inhibitors are selected from the following:
a) afatinib or nintedanib encapsulated alone;
b) afatinib and nintedanib co-encapsulated;
c) abemaciclib or sunitinib encapsulated alone;
d) abemaciclib and sunitinib co-encapsulated;
e) dasatinib or afatinib encapsulated alone;
f) dasatinib and afatinib co-encapsulated;
g) ceritinib or afatinib encapsulated alone;
h) ceritinib and afatinib co-encapsulated;
i) osimertinib or afatinib alone;
j) osimertinib and afatinib co-encapsulated;
k) osimertinib or crizotinib alone;
l) osimertinib and crizotinib co-encapsulated;
m) dasatinib or ceritinib encapsulated alone;
n) dasatinib and ceritinib co-encapsulated;
o) afatinib or crizotinib alone;
p) afatinib and crizotinib co-encapsulated;
q) afatinib and nintedanib in about 30:1 to about 1:30 molar ratio;
r) abemaciclib and sunitinib in about 30:1 to about 1:30 molar ratio;
s) dasatinib and afatinib in about 30:1 to about 1:30 molar ratio;
t) ceritinib and afatinib in about 30:1 to about 1:30 molar ratio;
u) osimertinib and afatinib in about 30:1 to about 1:30 molar ratio;
v) osimertinib and crizotinib in about 30:1 to about 1:30 molar ratio;
w) ceritinib and dasatinib in about 30:1 to about 1:30 molar ratio; and
x) afatinib and crizotinib in about 30:1 to about 1:30 molar ratio.

9. The pharmaceutical composition of claim 1, wherein the liposome dispersion liquid medium comprises water, a buffering agent, and a tonicity modifier, and optionally a control release excipient.

10. The pharmaceutical composition of claim 1, wherein pH of the liquid medium is in the range of about 5-8.

11. (canceled)

12. The pharmaceutical composition of claim 1, wherein molar ratio of the two or more co-encapsulated protein kinase inhibitors is such that when the ratio is provided to cancer cells relevant to the cancer in an in-vitro assay over a concentration range at which the fraction of affected cells is about 0.20 to 0.80 (i.e., about 20% to 80%), synergy is exhibited over at least 20% of the range.

13. The pharmaceutical composition of claim 1, wherein the liposome encapsulated with two or more protein kinase inhibitors maintains synergistic molar drug ratio in blood for at least one hour after in-vivo administration.

14. The pharmaceutical composition claim 1, wherein the interior compartment of the liposome further comprises a trapping agent when a protein kinase inhibitor is encapsulated within the aqueous interior core.

15. The pharmaceutical composition of claim 14, wherein the trapping agent is selected from ammonium sulfate, ammonium or substituted ammonium salts of polyanionized sulfobutyl ether cyclodextrin, ammonium or substituted ammonium salts of polyanionized sulfated carbohydrates, ammonium or substituted ammonium salts of polyphosphate, transition metal salts, quaternary ammonium compounds, polyoxyethylene (i.e., polyethylene glycols), coconut amine, and combinations thereof.

16. A drug loading method of preparing liposomes encapsulated with protein kinase inhibitor(s) which are only located within aqueous interior core of the liposomes, comprising the steps of:
(a) forming a lipid dispersion in a solution comprising a trapping agent selected from ammonium sulfate, ammonium or substituted ammonium salts of polyanionized sulfobutyl ether cyclodextrin, ammonium or substituted ammonium salts of polyanionized sulfated carbohydrates, ammonium or substituted ammonium salts of polyphosphate, (transition metal salts, quaternary ammonium compounds, polyoxyethylene (i.e., polyethylene glycols), coconut amine, and combinations thereof,
(b) reducing liposome particle size by extruding, sonicating or homogenizing the lipid dispersion at an elevated temperature;
(c) substantially removing the trapping agent outside of the liposome;
(d) warming the unloaded liposomes at an elevated temperature with a solution comprising one, two or more protein kinase inhibitor(s), thereby forming the drug loaded liposomes;
(e) adjusting the pH of the composition to about 5-8; and
(f) optionally, forming dry form of the product by lyophilization.

17. A drug loading method of preparing liposomes encapsulated with protein kinase inhibitor(s) within only hydrophobic lipid bilayer membrane of the liposomes, comprising the steps of:
(a) forming a lipid solution comprising one, two or more protein kinase inhibitor(s) in organic solvent(s);
(b) removing the organic solvent(s) and hydrating the lipid/drug mixture in an aqueous solution to form liposomes;
(c) reducing particle sizes of the liposomes by extruding, sonicating or homogenizing the lipid dispersion at an elevated temperature to form a composition;
(d) adjusting pH of the composition to about 5-8; and
(e) optionally, forming dry form of the product by lyophilization.

18. A method of preparing liposomes encapsulated with protein kinase inhibitor(s) in both aqueous interior core and lipid bilayer membrane of the liposomes, comprising the steps of:
(a) forming a lipid solution comprising one or more protein kinase inhibitors in organic solvent(s);
(b) removing the organic solvent(s) and hydrating the lipid/drug mixture in a solution containing a trapping agent to form liposomes, wherein the trapping agent is selected from ammonium sulfate, ammonium or substituted ammonium salts of polyanionized sulfobutyl ether cyclodextrin, ammonium or substituted ammonium salts of polyanionized sulfated carbohydrates, ammonium or substituted ammonium salts of polyphosphate, (transition metal salts, quaternary ammonium compounds, polyoxyethylene (i.e., polyethylene glycols), coconut amine, and combinations thereof;
(c) reducing particle sizes of the liposomes by extruding, sonicating or homogenizing the lipid dispersion at an elevated temperature;
(d) substantially removing the trapping agent outside of the liposome;
(e) warming the above liposome dispersion at an elevated temperature with a solution comprising at least one protein kinase inhibitor;
(f) adjusting the pH of the composition to about 5-8; and
(g) optionally, forming dry form of the product by lyophilization.

19. A method of treating a cancer, comprising administering to a subject in need of treatment a therapeutically effective amount of a pharmaceutical composition according to claim 1.

20. (canceled)

21. The method of claim 19, which has reduced drug side effects and drug resistance as compared to administration of the kinase inhibitors in a dosage form without liposomes, wherein the cancer is non-small cell lung cancer, colorectal cancer, renal cell cancer, breast cancer, gastrointestinal cancer, lung cancer, colorectal cancer, Ewing sarcoma, pancreatic cancer, prostate cancer, bladder cancer, kidney cancer, thyroid cancer, uterine cancer, gastrointestinal stromal tumors or others.

22. A treatment kit comprising a container and a plurality of the drug-loaded liposomes as described in claim 1 in the container, wherein the drug-loaded liposomes are or can be suspended in a sterile diluent solution ready for administration to a subject in need of treatment.
